(19) United States
(12) Patent Application Publication
(10) Pub. No.: US 2023/0251245 A1
(43) Pub. Date: Aug. 10, 2023

(54) Methods of Using Multi-Tissue Organoids

(71) Applicant: The University of Chicago, Chicago, IL (US)

(72) Inventors: Katherine L. Rhodes, South Hadley, MA (US); Kenneth A. Barr, Chicago, IL (US); Yoav Gilad, Chicago, IL (US)

(73) Assignee: The University of Chicago, Chicago, IL (US)

(21) Appl. No.: 18/067,192

(22) Filed: Dec. 16, 2022

Related U.S. Application Data
(60) Provisional application No. 63/291,045, filed on Dec. 17, 2021.

(51) Int. Cl. G01N 33/50; C12Q 1/6809; G16B 5/30

(52) U.S. Cl. CPC G01N 33/5082; C12Q 1/6809; G16B 5/30; G01N 2570/00

(57) ABSTRACT

In aspects, the present disclosure provides methods of using multi-tissue organoids bodies including for analyzing the genome or transcriptome of a mammal, analyzing a cellular response to a treatment, determining an effect of a substance, or analyzing a cellular response to a treatment.

### CROSS-REFERENCE TO RELATED APPLICATION

This patent application claims the benefit of U.S. Provisional Patent Application No. 63/291,045 filed Dec. 17, 2021, which is incorporated by reference herein in its entirety.

### STATEMENT REGARDING FEDERALLY SPONSORED RESEARCH OR DEVELOPMENT

This invention was made with government support under grant number CA014599 awarded by the National Institutes of Health. The government has certain rights in the invention.

This invention was made with government support under grant numbers R35GM131726 and F31HL146171 awarded by the National Institutes of Health. The government has certain rights in the invention.

### BACKGROUND

Genome-wide association studies (GWAS) have identified thousands of genetic variants associated with human traits and diseases, many of which are located in noncoding regions of the genome and are putatively regulatory in function. Molecular assays may be used to understand regulatory and functional effects of trait-associated variants in cell types during stages of development and potentially to also model different environmental exposures. However, most efforts to identify genetic variants that regulate gene expression (expression quantitative trait loci, or eQTLs) have relied on adult tissue samples collected at a single time point or induced pluripotent stem cells (iPSCs), both of which are limited in their potential for identifying dynamic regulatory effects.

There is an ongoing need for methods to analyze static and dynamic regulatory effects, as well as transcriptomic variation at the cellular level.

### BRIEF SUMMARY

In aspects, the present disclosure provides a method of analyzing the genome or transcriptome of a mammal, the method comprising, consisting essentially of, or consisting of: (a) forming a first multi-tissue organoid from a first pluripotent stem cell (PSC) from a first mammal; (b) forming a second multi-tissue organoid from a second PSC from a second mammal, wherein the second mammal is of the same species as the first mammal or of a different species than the first mammal; (c) permitting each of the first multi-tissue organoid and the second multi-tissue organoid to differentiate; (d) identifying cells within the first multi-tissue organoid and the second multi-tissue organoid by (1) clustering cells and annotating the cells based on the genes that are highly expressed in each cluster, (2) annotating cell type by correlation of gene expression using a reference data set of known primary cell types, or annotating cell state by correlation of gene expression using a reference data set of genes involved in known cells states, and (3) applying topic modeling, matrix factorization, or grades of membership modelling to the annotated cells; (e) performing single cell analysis on the first multi-tissue organoid and on the second multi-tissue organoid to generate data for the genome or transcriptome of the first multi-tissue organoid and to generate data for the genome or transcriptome of the second multi-tissue organoid, the generated data being for the genome of both the first multi-tissue organoid and the second multi-tissue organoid or for the transcriptome of both the first multi-tissue organoid and the second multi-tissue organoid; and (f) comparing the data of the first multi-tissue organoid to the data of the second multi-tissue organoid.

In aspects, the present disclosure provides a method of analyzing a cellular response to a treatment, the method comprising, consisting essentially of, or consisting of: (a) forming a first multi-tissue organoid from a first pluripotent stem cell (PSC) from a first mammal; (b) forming a second multi-tissue organoid from a second PSC from the first mammal; (c) permitting each of the first multi-tissue organoid and the second multi-tissue organoid to differentiate; (d) identifying cells within the first multi-tissue organoid and the second multi-tissue organoid by

(1) clustering cells and annotating the cells based on the genes that are highly expressed in each cluster, (2) annotating cell type by correlation of gene expression using a reference data set of known primary cell types, or annotating cell state by correlation of gene expression using a reference data set of genes involved in known cells states, and (3) applying topic modeling, matrix factorization, or grades of membership modelling to the annotated cells; (e) treating the first multi-tissue organoid with a substance while not treating the second multi-tissue organoid with the substance; and (f) comparing the treatment of the first multi-tissue organoid to the untreated second multi-tissue organoid.

In aspects, the present disclosure provides a method of determining an effect of a substance, the method comprising, consisting essentially of, or consisting of: (a) forming two or more multi-tissue organoids, each organoid formed from a separate pluripotent stem cell (PSC) from a mammal; (b) permitting each of the multi-tissue organoids to differentiate; (c) identifying cells within each of the multi-tissue organoids by (1) clustering cells and annotating the cells based on the genes that are highly expressed in each cluster, (2) annotating cell type by correlation of gene expression using a reference data set of known primary cell types, or annotating cell state by correlation of gene expression using a reference data set of genes involved in known cells states, and (3) applying topic modeling, matrix factorization, or grades of membership modelling to the annotated cells; (d) treating the two or more multi-tissue organoids with an amount of the substance, wherein each of the multi-tissue organoids is treated with a different amount of the substance; (f) comparing the two or more multi-tissue organoids; and (g) determining an effect of the substance on the two or more multi-tissue organoids.

In aspects, the present disclosure provides a method of analyzing a cellular response to a treatment, the method comprising, consisting essentially of, or consisting of: (a) forming a first multi-tissue organoid from a first pluripotent stem cell (PSC) from a first mammal, wherein the first mammal has a disease-state; (b) forming a second multi-tissue organoid from a second PSC from a second mammal, wherein the second mammal is of the same species as the first mammal or of a different species than the first mammal and the second mammal does not have the disease-state; (c) permitting each of the first multi-tissue organoid and the second multi-tissue organoid to differentiate; (d) identifying cells within the first multi-tissue organoid and the second multi-tissue organoid by (1) clustering cells and annotating the cells based on the genes that are highly expressed in each cluster, (2) annotating cell type by correlation of gene expression using a reference data set of known primary cell types, or annotating cell state by correlation of gene expression using a reference data set of genes involved in known cells states, and (3) applying topic modeling, matrix factorization, or grades of membership modelling to the annotated cells; (e) treating the first multi-tissue organoid and the second multi-tissue organoid with a substance; and (f) comparing the treatment of the first multi-tissue organoid to the treatment of the second multi-tissue organoid.

Additional aspects of the present disclosure are as described herein.

### BRIEF DESCRIPTION OF THE DRAWINGS

FIGS. 1A-1F are uniform manifold approximation and projection (UMAP) visualizations of multi-tissue organoid (MTO) cells from human lines 18511, 18858, and 19160 colored by expression of POU5F1 (FIG. 1A), SOX17 (FIG. 1B), HAND1 (FIG. 1C), PAX6 (FIG. 1D), and proportional stacked bar graphs of cell types from replicates of lines 18511, 18858, and 19160 colored by Seurat cluster assignment at clustering resolution 0.1 (FIG. 1E) and from cell lines 18856, 18912, 19140, 19159, and 19210 (FIG. 1F).

FIG. 2 is a UMAP visualizations of MTO cells from human lines 18511, 18858, and 19610, and cells from the fetal reference after integration.

FIGS. 3A-3D are a UMAP visualization of cells colored by loading Topic 1 (FIG. 3A), and box plots showing the loading of Topic 1 from the k=6 topic analysis on each Seurat cluster at clustering resolution 0.1 (FIG. 3B) and at clustering resolution 1 (FIG. 3C), and a volcano plot showing differentially expressed (DE) genes between Topic 1 and all other topics from the k=6 topic analysis on the logarithmic scale (FIG. 3D). Cells are from human lines 18511, 18858, and 19160.

FIGS. 4A-4C are a heatmap showing hierarchical clustering of cells based on normalized gene expression (FIG. 4A), and violin plots showing percent of variance in gene expression explained by cluster (resolution 0.1), replicate, and individual in this data set after partitioning variance in pseudobulk samples (FIG. 4B) and after partitioning variance at single-cell sample resolution (FIG. 4C). Cells are from human lines 18511, 18858, and 19160.

FIGS. 5A-5E are graphs of power curves showing ability to detect expression quantitative trait loci (eQTLs) where the horizontal red line represents a power to detect eQTLs of 0.8 when n=3 (FIG. 5A), n=10 (FIG. 5B), n=30 (FIG. 5C), n=58 (FIG. 5D), n=100 (FIG. 5E). Data are from human lines 18511, 18858, and 19160.

FIGS. 6A-6F are partition based graph abstraction (PAGA) visualizations where the numbers represent Seurat clusters identified at clustering resolution 1 and the trajectories were manually drawn based on expression of marker genes for each trajectory showing the neuronal lineage (FIG. 6A), the hepatic lineage (FIG. 6B), and the endothelial lineage (FIG. 6C), and heatmaps showing the frequency with which individual-replicate groups were assigned to the same cluster after running split-GPM 10 times in the neuronal (FIG. 6D), hepatic (FIG. 6E), and endothelial (FIG. 6F) lineages. Data are from human lines 18511, 18858, and 19160.

FIGS. 7A-7B are violin plots for the cells of each individual of each replicate after filtering showing the total unique molecular identifier (UMI) counts (FIG. 7A) and the number of genes (features) expressed (FIG. 7B). Data are from human lines 18511, 18858, and 19160.

FIGS. 8A-8C are quality control metrics for each of the human cell lines 18856, 18912, 19140, 19159, and 19210 after filtering showing the number of genes (features) expressed (FIG. 8A), the total unique molecular identifier (UMI) counts (FIG. 8B), and the percentage (FIG. 8C).

FIG. 9 is a UMAP visualization of MTO cells from human lines 18511, 18858, and 19160 and cells from each reference set after integration of separated data set.

FIGS. 10A-10C are volcano plots of all tested genes, that show labeled DE genes in reference annotated MTO cell types in cells from human lines 18511, 18858, and 19160 that show annotated cardiomyocytes compared to all other cell types with cardiomyocyte marker genes MYL7, MYL4, and TNNT2 labeled (FIG. 10A), annotated hepatoblasts compared to all other cell types with hepatoblast marker genes AFP, FGB, and ACSS2 labeled (FIG. 10B), and annotated mesothelial cells compared to all other cell types with mesothelial marker genes NID2, COL1A1, COL6A3, COL3A1, and COL6A1 labeled (FIG. 10C).

FIGS. 11A-11F are UMAP visualizations of k=6 topic loading showing Topic 1 (FIG. 11A), Topic 2 (FIG. 11B), Topic 3 (FIG. 11C), Topic 4 (FIG. 11D), Topic 5 (FIG. 11E), and Topic 6 (FIG. 11F). Data are from human lines 18511, 18858, and 19160.

FIGS. 12A-12F are volcano plots of the genes driving each topic from the k=6 topic analysis that show Topic 1 (FIG. 12A), Topic 2 (FIG. 12B), Topic 3 (FIG. 12C), Topic 4 (FIG. 12D), Topic 5 (FIG. 12E), and Topic 6 (FIG. 12F). Points are colored by the average count on the logarithmic scale, and the top 10 driver genes of each topic are labeled. Data are from human lines 18511, 18858, and 19160.

FIGS. 13A-13D are bar plots showing the loading of each topic from the k=6 analysis on each Seurat cluster at resolution 0.1 (FIG. 13A), resolution 0.5 (FIG. 13B), resolution 0.8 (FIG. 13C), and resolution 1 (FIG. 13D). Data are from human lines 18511, 18858, and 19160.

FIGS. 14A-14D are hierarchical clustering of samples' individual-replicate groups by the proportions of cells from each group assigned to each Seurat cluster at resolution 0.1 (FIG. 14A), resolution 0.5 (FIG. 14B), resolution 0.8 (FIG. 14C), and resolution 1 (FIG. 14D). Data are from human lines 18511, 18858, and 19160.

FIGS. 15A-15E are hierarchical clustering of samples' individual-replicate groups by the loading of each topic with k=6 topics (FIG. 15A), k=10 topics (FIG. 15B), k=15 topics (FIG. 15C), k=25 topics (FIG. 15D), and k=30 topics (FIG. 15E). Data are from human lines 18511, 18858, and 19160.

FIGS. 16A-16C are violin plots showing the percent of variance in gene expression explained by cluster, replicate, and individual in this data set after partitioning variance in pseudobulk samples when the clustering is at resolution 0.5 (FIG. 16A), resolution 0.8 (FIG. 16B), and resolution 1 (FIG. 16C). Data are from human lines 18511, 18858, and 19160.

FIGS. 17A-17G are violin plots showing the percent of variance in gene expression explained by replicate and individual in each Seurat cluster (clustering resolution 0.1) for cluster 0 of pluripotent cells (FIG. 17A), cluster 1 of early ectoderm (FIG. 17B), cluster 2 of mesoderm (FIG. 17C), cluster 3 of neural crest (FIG. 17D), cluster 4 of endoderm (FIG. 17E), cluster 5 of neurons (FIG. 17F), and cluster 6 of endothelia (FIG. 17G). Data are from human lines 18511, 18858, and 19160.

FIG. 18 is a bar graph showing the median percent of variance explained by replicate (black bars) and individual (white bars) in each Seurat cluster using pseudobulk samples. Data are from human lines 18511, 18858, and 19160.

FIGS. 19A-19C are a force atlas plot of MTO cells colored by broad cell type categories corresponding to Seurat clusters as shown in Table 2, identified at clustering resolution 0.1 (FIG. 19A), and a PAGA graph showing inferred edges between Seurat clusters defined at clustering resolution 1 (FIG. 19B), and a force atlas plot of diffusion pseudotime values (FIG. 19C). Data are from human lines 18511, 18858, and 19160.

FIGS. 20A-20C are dynamic expression patterns of identified gene modules in each cluster of replicate-individual samples for the neuronal lineage (FIG. 20A), hepatic lineage (FIG. 20B), and endothelial lineage (FIG. 20C). Data are from human lines 18511, 18858, and 19160.

FIGS. 21A-J are a collection of figures that explore differential expression of genes between species across cell types and the relationship between differential expression and measures of genomic evolution: a heatmap of correlation motifs across cell types from human and chimpanzee cell lines, with cells shaded according to the posterior probability of differential expression for each motif and cell type between species, and with columns clustered by mean gene expression (FIG. 21A), and boxplots of percent coding identity (FIG. 21B), dN/dS (FIG. 21C), and LOEUF in genes that are DE in zero or increasingly large numbers of cell types at posterior probability 0.5 (FIG. 21D), and a boxplot showing the distribution of the number of cell types in which genes are DE for HAR targets and non-HAR target genes (FIG. 21E), and the number of non-DE genes remaining, requiring the specified absolute expression cutoffs in at least the given number of cell types (FIG. 21F), and UMAP visualizations colored to show the posterior probability of DE for genes in motif 13 (FIG. 21G) and the expression z-score of genes in motif 13 (FIG. 21H), and boxplots showing expression relative to humans in select cell types for TCF7L2 (FIG. 21I) and SCG3 (FIG. 21J).

### DETAILED DESCRIPTION

In aspects, the present disclosure provides a method of analyzing the genome or transcriptome of a mammal, the method comprising, consisting essentially of, or consisting of: (a) forming a first multi-tissue organoid from a first pluripotent stem cell (PSC) from a first mammal; (b) forming a second multi-tissue organoid from a second PSC from a second mammal, wherein the second mammal is of the same species as the first mammal or of a different species than the first mammal; (c) permitting each of the first multi-tissue organoid and the second multi-tissue organoid to differentiate; (d) identifying cells within the first multi-tissue organoid and the second multi-tissue organoid by (1) clustering cells and annotating the cells based on the genes that are highly expressed in each cluster, (2) annotating cell type by correlation of gene expression using a reference data set of known primary cell types, or annotating cell state by correlation of gene expression using a reference data set of genes involved in known cells states, and (3) applying topic modeling, matrix factorization, or grades of membership modelling to the annotated cells; (e) performing single cell analysis on the first multi-tissue organoid and on the second multi-tissue organoid to generate data for the genome or transcriptome of the first multi-tissue organoid and to generate data for the genome or transcriptome of the second multi-tissue organoid, the generated data being for the genome of both the first multi-tissue organoid and the second multi-tissue organoid or for the transcriptome of both the first multi-tissue organoid and the second multi-tissue organoid; and (f) comparing the data of the first multi-tissue organoid to the data of the second multi-tissue organoid.

The term “multi-tissue organoid” used herein refers to a three dimensional collection of cells, derived from an iPSC or embryonic stem cell (ESC), that comprises spatially and temporally diverse cell types, including cells from each germ layer and cells representing multiple stages of development across distinct developmental trajectories.

The term “clustering cells” used herein refers to grouping similar cells, often, but not always, with similar gene expression profiles. Clustering can be performed, for example, by a clustering analysis algorithm, such as the Louvain algorithm in Seurat. Clustering using such an algorithm can be performed at various resolutions, for example, at 0.1, 0.5, 0.8, or 1.

The term “annotating the cells” used herein refers to characterizing cells as belonging to a particular cell type or cell state. Cell types include, but are not limited to: pluripotent cell, early ectodermal cell, endothelial cell, hepatocytes, mesodermal cells, neural crest cells, neurons, acinar cells, adrenocortical cells, AFP_ALB positive cells, amacrine cells, antigen presenting cells, astrocytes, bipolar cells, cardiomyocytes, CCL19_CCL21 positive cells, chromaffin cells, ciliated epithelial cells, CLC_IL5RA positive cells, corneal and conjunctival epithelial cells, ductal cells, ELF3_AGBL2 positive cells, endocardial cells, ENS glia, ENS neurons, epicardial fat cells, erythroblasts, excitatory neurons, ganglion cells, goblet cells, granule neurons, hematopoietic stem cells, hepatoblasts, horizontal cells, IGFBP1_DKK1 positive cells, inhibitory interneurons, inhibitory neurons, intestinal epithelial cells, islet endocrine cells, lens fibre cells, limbic system neurons, lymphatic endothelial cells, megakaryocytes, mesangial cells, mesothelial cells, metanephric cells, microglia, MUC13_DMBT1 positive cells, myeloid cells, neuroendocrine cells, oligodendrocytes, parietal and chief cells, PDE11A_FAM19A2 positive cells, photoreceptor cells, Purkinje neurons, retinal pigment cells, retinal progenitors and muller glia, scHCL.hESC, Schwann cells, skeletal muscle cells, SKOR2_NPSR1 positive cells, SLC24A4_PEX5L positive cells, smooth muscle cells, squamous epithelial cells, stellate cells, stromal cells, syncytiotrophoblasts, villous cytotrophoblasts, thymic epithelial cells, thymocytes, trophoblast giant cells, unipolar brush cells, ureteric bud cells, vascular endothelial cells, visceral neurons, and others. Annotating cell type can be performed, for example, by correlating gene expression data using a reference data set, such as the CelliD method (Cortal et al., 2021). The reference data set can include but is not limited to reference sets of: primary fetal cell types, Day 20 hESC-derived MTOs, and hESCs. Annotating cell type can also be performed by identifying upregulated canonical marker genes by clustering as described above, performing differential expression to find genes upregulated in each cluster compared to all other clusters, and then noting significantly increased expression of known marker genes. Cell states, include, but are not limited to: cell cycle stage, differentiation stage, or any other factor that can alter a cell. Annotating cell state can be performed using a single cell analysis tool, such as Seurat or Scanpy, based on the expression of a category of genes such as cell cycle genes or, genes involved in differentiation.

The term “topic modeling” used herein refers to a process of learning major patterns in gene expression within a data set, or topics, and modeling each cell as a combination of these topics. This process is sometimes referred to as aspect modeling, probabilistic latent semantic indexing (pLSI), or latent Dirichlet allocation (LDA). This process allows for cells to be characterized by grades of membership to multiple cell categories. Topic modeling can use non-negative matrix factorization algorithms for data analysis. Topic modeling can be performed using programs, including but not limited to: fastTopics and Multi-Omics Factor Analysis (MOFA).

Single cell analysis methods can be used to generate data for a genome or transcriptome using techniques such as: single cell RNA sequencing (sc-RNA-seq); single-cell assay for transposase-accessible chromatin using sequencing (scATAC-seq); spatial transcriptomics, fluorescence in situ hybridization (FISH); single molecule RNA FISH (smFISH); quantitative FISH (Q-FISH); Flow-FISH; microfluidics-assisted FISH (MA-FISH); microautoradiography FISH (MAR-FISH); hybrid fusion FISH (HF-FISH); multiplexed error-robust FISH (MERFISH); cellular indexing of transcriptomes and epitopes by sequencing (CITE-seq); fluorescence-activated cell sorting (FACS); lineage tracing by nuclease-activated editing of ubiquitous sequences (LINNAEUS); massively parallel RNA single-cell sequencing (MARS-seq); memory by engineered mutagenesis with optical in situ readout (MEMOIR); proximity extension assay (PEA); RNA expression and protein sequencing assay (REAP-seq); single-cell bisulfate sequencing (scBS-seq); single-cell chromatin immunoprecipitation followed by sequencing (scChIP-seq); single-cell genome editing of synthetic target arrays for lineage tracing (scGESTALT); single-cell combinatorial indexing for methylation analysis (sci-MET); single-cell combinatorial indexing RNA sequencing (sci-RNA-seq); single-cell combinatorial indexed sequencing (SCI-seq); single-cell combinatorial indexing assay for transposase-accessible chromatin using sequencing (sciATAC-seq); single-cell transposome hypersensitivity site sequencing (scTHS-seq); single-nucleus methylcytosine sequencing (snmC-seq); single-nucleus sequencing (SNS); split-pool ligation-based transcriptome sequencing (SPLiT-seq); spatially resolved transcript amplicon readout mapping (STARmap); spatial transcriptomics; Visium spatial gene expression; slide-seq; high definition spatial transcriptomics (HDST), GeoMX digital spatial profiler (DSP), or any other type of single-cell assay. In aspects the single cell analysis is single cell RNA-sequencing (scRNA-seq). In aspects the single cell analysis is single cell assay for transposase accessible chromatin (scATAC-seq).

Comparing data of multi-tissue organoids can include, but is not limited to identifying genetic variation using whole genome sequencing, identifying variation in the transcriptome using differential expression analysis, identifying variation in the chromatin landscape using differential chromatin accessibility, and identifying variation in cell type composition.

Pluripotent stem cells (PSC) are cells capable of differentiating into any cell type derived from the ectoderm, mesoderm, or endoderm germ layer. Induced pluripotent stem cells (iPSC) are derived from a non-pluripotent cell, such as a somatic cell, by the introduction of transcription factors. In aspects the first PSC or the second PSC is an induced PSC (iPSC). In aspects the first PSC and second PSC are each an iPSC.

The term “quantitative trait locus” (QTL) used herein refers to a DNA region, or locus, that explains a portion of the genetic variance of a phenotype. QTLs can be further categorized by their molecular phenotype into categories including, but not limited to gene expression QTLs (eQTL), chromatin accessibility QTLs (caQTL), and splicing QTLs (sQTL). eQTLs can be identified by identifying an association between genotype at a specific locus, such as a small nucleotide polymorphism (SNP) and differences in gene expression. Existing eQTL data sets can also be used to identify an eQTL, including, but not limited to: GTEx data set, Strober et al. data set, and eQTL Catalogue data set. In aspects the data identifies association of a genetic variant with a molecular phenotype. In aspects the data identifies an expression quantitative trait loci (eQTL).

The mammal may be any suitable mammal. Mammals include, but are not limited to, the order Rodentia, such as mice, and the order Lagomorpha, such as rabbits. The mammal can be from the order Carnivora, including Felines (cats) and Canines (dogs). The mammal can be from the order Artiodactyla, including Bovines (cows) and Swines (pigs) or of the order Perissodactyla, including Equines (horses). The mammal can be of the order Primates, Cebids, or Simioids (monkeys) or of the order Anthropoids (humans and apes). In aspects the mammals are primates. In aspects the primates are each human.

In aspects the present disclosure provides methods as described herein, wherein the method further comprises, consists essentially of, or consists of: (i) forming a third multi-tissue organoid from a third PSC from a third mammal, wherein the third mammal is of the same species as the first mammal and the second mammal or of a different species than the first mammal and the second mammal; (ii) permitting the third multi-tissue organoid to differentiate; (iii) identifying cells within the third multi-tissue organoid by (1) clustering cells and annotating the cells based on the genes that are highly expressed in each cluster, (2) annotating cell type by correlation of gene expression using a reference data set of known primary cell types, or annotating cell state by correlation of gene expression using a reference data set of genes involved in known cells states, and (3) applying topic modeling, matrix factorization, or grades of membership modelling to the annotated cells; (iv) performing single cell analysis on the third multi-tissue organoid to generate data for the genome or transcriptome of the third multi-tissue organoid, the generated data being for the genome of each of the first multi-tissue organoid, the second multi-tissue organoid, and the third multi-tissue organoid, or for the transcriptome of each of the first multi-tissue organoid, the second multi-tissue organoid, and the third multi-tissue organoid; and (v) comparing the data of the third multi-tissue organoid to the data of the first multi-tissue organoid and to the data of the second multi-tissue organoid. In aspects identifying cells comprise, consist essentially of, or consist of applying topic modeling.

In aspects, the present disclosure provides a method of classifying mammals, the method comprising, consisting essentially of, or consisting of: (A) analyzing the genome or transcriptome of two or more mammals according to the methods described above, wherein the analysis identifies a response expression quantitative trait loci (response eQTL); and (B) classifying the two or more mammals based on the response eQTL. When single-cell RNA sequencing is used, response QTLs can be called using a variety of differing techniques. For example, eQTLs can be called using pseudobulk data from cells assigned to a cell type via clustering and marker gene analysis. Or, eQTLs can be called using gene expression measurements from individual cells. In cases where a single genetic variant has a large effect on drug toxicity, presence or absence of that variant can be used to stratify the ideal patient population and to inform decisions about which patients receive the drug or what dose will be most appropriate.

Often, many genetic variants will have a small effect on drug response. Using the effect sizes of response eQTL analysis, a Polygenic Risk Score (PRS) can be calculated, representing an individual's likelihood for drug toxicity or efficacy based on the individual's genetic background. PRS can then be used to inform treatment decisions or to recruit the ideal patient population for clinical trials.

In aspects, the present disclosure provides a method of analyzing a disease-state of a mammal, the method comprising, consisting essentially of, or consisting of analyzing a genome or transcriptome of a mammal according to the methods described above, wherein the genome or transcriptome is associated with a disease-state.

In aspects the data generated is for the genome of each of the multi-tissue organoids. In aspects the data generated is for the transcriptome of each of the multi-tissue organoids.

In aspects, the present disclosure provides a method of analyzing a cellular response to a treatment, the method comprising, consisting essentially of, or consisting of: (a) forming a first multi-tissue organoid from a first pluripotent stem cell (PSC) from a first mammal; (b) forming a second multi-tissue organoid from a second PSC from the first mammal; (c) permitting each of the first multi-tissue organoid and the second multi-tissue organoid to differentiate; (d) identifying cells within the first multi-tissue organoid and the second multi-tissue organoid by (1) clustering cells and annotating the cells based on the genes that are highly expressed in each cluster, (2) annotating cell type by correlation of gene expression using a reference data set of known primary cell types, or annotating cell state by correlation of gene expression using a reference data set of genes involved in known cells states, and (3) applying topic modeling, matrix factorization, or grades of membership modelling to the annotated cells; (e) treating the first multi-tissue organoid with a substance while not treating the second multi-tissue organoid with the substance; and (f) comparing the treatment of the first multi-tissue organoid to the untreated second multi-tissue organoid.

A “substance” as used herein is any substance, for example, a small molecule, nucleic acid such as DNA or RNA, an antibody, peptides, or a large molecule such as a protein.

The terms “treated,” “treating,” “treatment,” “optimal dosage,” “on-target therapeutic effects,” etc. used herein do not necessarily imply 100% or complete treatment. Rather, there are varying degrees, which one of ordinary skill in the art recognizes as having a potential benefit or therapeutic effect. In this respect, the substance and methods can provide any amount of any level of treatment. Furthermore, the treatment provided by the disclosed method can include the treatment of one or more conditions or symptoms of the disease or condition being treated.

Toxicity of a substance can be assessed by identifying cell type specific patterns of cell death and cell type specific upregulation of genes associated with toxicity, including genes associated with stress and apoptosis. Patterns of gene expression associated with cell type specific drug toxicity can be learned by collecting single-cell RNA-seq data from multi-tissue organoids treated with drugs with known toxicities in vivo and extracting insights, e.g., by using machine learning algorithms.

In aspects the present disclosure provides methods as described herein, wherein the methods further comprise, consist essentially of, or consist of: (i) forming a third multi-tissue organoid from a third pluripotent stem cell (PSC) from a second mammal, wherein the second mammal is of the same species as the first mammal or of a different species than the first mammal; (ii) forming a fourth multi-tissue organoid from a fourth PSC from the second mammal; (iii) permitting each of the third multi-tissue organoid and the fourth multi-tissue organoid to differentiate; (iv) identifying cells within the third multi-tissue organoid and the fourth multi-tissue organoid by (1) clustering cells and annotating the cells based on the genes that are highly expressed in each cluster, (2) annotating cell type by correlation of gene expression using a reference data set of known primary cell types, or annotating cell state by correlation of gene expression using a reference data set of genes involved in known cells states, and (3) applying topic modeling, matrix factorization, or grades of membership modelling to the annotated cells; (v) treating the third multi-tissue organoid with a substance while not treating the fourth multi-tissue organoid with the substance; and (vi) comparing the treatment of the third multi-tissue organoid to the untreated fourth multi-tissue organoid.

In aspects the present disclosure provides methods as described herein, wherein the methods further comprise, consist essentially of, or consist of: comparing (1) the treatment of the first multi-tissue organoid compared to the untreated second multi-tissue organoid to (2) the treatment of the third multi-tissue organoid compared to the untreated fourth multi-tissue organoid.

In aspects, the present disclosure provides a method of determining an effect of a substance, the method comprising, consisting essentially of, or consisting of: (a) forming two or more multi-tissue organoids, each organoid formed from a separate pluripotent stem cell (PSC) from a mammal; (b) permitting each of the multi-tissue organoids to differentiate; (c) identifying cells within each of the multi-tissue organoids by (1) clustering cells and annotating the cells based on the genes that are highly expressed in each cluster, (2) annotating cell type by correlation of gene expression using a reference data set of known primary cell types, or annotating cell state by correlation of gene expression using a reference data set of genes involved in known cells states, and (3) applying topic modeling, matrix factorization, or grades of membership modelling to the annotated cells; (d) treating the two or more multi-tissue organoids with an amount of the substance, wherein each of the multi-tissue organoids is treated with a different amount of the substance; (f) comparing the two or more multi-tissue organoids; and (g) determining an effect of the substance on the two or more multi-tissue organoids.

Results from dose-range studies can inform dosing and toxicity testing in vivo. For example, if cell-type specific toxicity arises at particular doses, animal studies could be conducted to include specific testing of the affected cell types or tissues.

In aspects the present disclosure provides methods as described herein, wherein the methods further comprise, consist essentially of, or consist of: (i) forming an additional multi-tissue organoid from an additional PSC from the mammal; (ii) permitting the additional multi-tissue organoid to differentiate; (iii) identifying cells within the additional multi-tissue organoid by (1) clustering cells and annotating the cells based on the genes that are highly expressed in each cluster, (2) annotating cell type by correlation of gene expression using a reference data set of known primary cell types, or annotating cell state by correlation of gene expression using a reference data set of genes involved in known cells states, and (3) applying topic modeling, matrix factorization, or grades of membership modelling to the annotated cells; (iv) leaving the additional multi-tissue organoid untreated with the substance; and (v) comparing the two or more multi-tissue organoids to the additional multi-tissue organoid.

In aspects comparing the two or more multi-tissue organoids to the additional multi-tissue organoid comprises, consists essentially of, or consists of assessing in at least one of the multi-tissue organoids cell death of one or more cell types. In aspects comparing the two or more multi-tissue organoids to the additional multi-tissue organoid comprises, consists essentially of, or consists of assessing all cell types within the multi-tissue organoids.

In aspects the present disclosure provides methods as described herein, wherein the methods further comprise, consist essentially of, or consist of: determining an optimal dosage, wherein the optimal dosage is the amount of the substance that is determined to cause reduced cell death and reduced stress response across all cell types and to cause on-target effects for a subset of cell types compared to other amounts of the substance.

The term “environmental compound” used herein refers to any compounds existing in air, water, food, or soil, including but not limited to environmental contaminants, such as microplastics, per- and polyfluoroalkyl substances (PFAs), lead, mercury, radon, pesticides, and pollutants. In aspects the substance is an environmental compound.

In aspects, the present disclosure provides a method of treating a multi-tissue organoid, the method comprising, consisting essentially of, or consisting of: (A) determining an optimal dosage of a substance according to the method of determining an effect described above; (B) forming another multi-tissue organoid from a PSC from a mammal; (C) permitting the multi-tissue organoid of (B) to differentiate; and (D) treating the differentiated multi-tissue organoid of (C) with the dosage determined in (A).

In aspects, the present disclosure provides a method of treating a mammal, the method comprising, consisting essentially of, or consisting of: (A) determining an optimal dosage of a substance according to the method of determining an effect described above; and (B) treating the mammal with the optimal dosage of the substance as determined in (A).

In aspects the present disclosure provides methods as described herein, wherein the methods further comprise, consist essentially of, or consist of: assessing in the treated mammal the health of a cell type that was adversely affected by the substance in a multi-tissue organoid during determination of the optimal dosage.

In aspects, the present disclosure provides a method of analyzing a cellular response to a treatment, the method comprising, consisting essentially of, or consisting of: (a) forming a first multi-tissue organoid from a first pluripotent stem cell (PSC) from a first mammal, wherein the first mammal has a disease-state; (b) forming a second multi-tissue organoid from a second PSC from a second mammal, wherein the second mammal is of the same species as the first mammal or of a different species than the first mammal and the second mammal does not have the disease-state; (c) permitting each of the first multi-tissue organoid and the second multi-tissue organoid to differentiate; (d) identifying cells within the first multi-tissue organoid and the second multi-tissue organoid by (1) clustering cells and annotating the cells based on the genes that are highly expressed in each cluster, (2) annotating cell type by correlation of gene expression using a reference data set of known primary cell types, or annotating cell state by correlation of gene expression using a reference data set of genes involved in known cells states, and (3) applying topic modeling, matrix factorization, or grades of membership modelling to the annotated cells; (e) treating the first multi-tissue organoid and the second multi-tissue organoid with a substance; and (f) comparing the treatment of the first multi-tissue organoid to the treatment of the second multi-tissue organoid.

In aspects the present disclosure provides methods as described herein, wherein the methods further comprise, consist essentially of, or consist of: (i) forming an additional multi-tissue organoid from an additional PSC from the first mammal or the second mammal; (ii) permitting the additional multi-tissue organoid to differentiate; (iii) identifying cells within the additional multi-tissue organoid by (1) clustering cells and annotating the cells based on the genes that are highly expressed in each cluster, (2) annotating cell type by correlation of gene expression using a reference data set of known primary cell types, or annotating cell state by correlation of gene expression using a reference data set of genes involved in known cells states, and (3) applying topic modeling, matrix factorization, or grades of membership modelling to the annotated cells; and (iv) leaving the additional multi-tissue organoid untreated with the substance; and (v) comparing the treatment of the first multi-tissue organoid and the treatment of the second multi-tissue organoid to the additional multi-tissue organoid.

In aspects the assessment comprises, consists essentially of, or consists of using microscopy, live/dead cell staining, immunostaining, or cell counting. In aspects the assessment comprises, consists essentially of, or consists of using flow cytometry, qPCR, or single cell sequencing. In aspects the assessment comprises, consists essentially of, or consists of assessing expression of genes associated with cell stress or apoptosis. In aspects assessing expression of genes associated with cell stress or apoptosis comprises, consists essentially of, or consists of using low-depth single cell sequencing or qPCR. In aspects the assessment comprises, consists essentially of, or consists of assessing on-target therapeutic effects on gene expression. In aspects assessing on-target therapeutic effects on gene expression comprises, consists essentially of, or consists of using low-depth single cell sequencing or qPCR.

In aspects, a multi-tissue organoid can be used in optimizing diffusion of a substance into a cell, tissue, or organoid. This may include treating a multi-tissue organoid with an enzyme such as collagenase to break up the outer layer or may involve treating a multi-tissue organoid with a combination of enzymes or culture conditions (e.g., spinner flasks).

In aspects, a multi-tissue organoid may be used in spatial transcriptomic analysis of multi-tissue organoids (MTOs) or spatial analysis of expression of one or a few genes or proteins within MTOs. It is contemplated that such analysis could determine localization of cell types and could be used to determine the effects of paracrine signaling on gene expression and analyze how gene expression in neighboring cells influence gene regulation in a cell.

The following are aspects of the disclosure.

1. A method of analyzing the genome or transcriptome of a mammal, the method comprising:
(a) forming a first multi-tissue organoid from a first pluripotent stem cell (PSC) from a first mammal;
(b) forming a second multi-tissue organoid from a second PSC from a second mammal, wherein the second mammal is of the same species as the first mammal or of a different species than the first mammal;
(c) permitting each of the first multi-tissue organoid and the second multi-tissue organoid to differentiate;
(d) identifying cells within the first multi-tissue organoid and the second multi-tissue organoid by
   (1) clustering cells and annotating the cells based on the genes that are highly expressed in each cluster,
   (2) annotating cell type by correlation of gene expression using a reference data set of known primary cell types, or annotating cell state by correlation of gene expression using a reference data set of genes involved in known cells states, and
   (3) applying topic modeling, matrix factorization, or grades of membership modelling to the annotated cells;
(e) performing single cell analysis on the first multi-tissue organoid and on the second multi-tissue organoid to generate data for the genome or transcriptome of the first multi-tissue organoid and to generate data for the genome or transcriptome of the second multi-tissue organoid,
   the generated data being for the genome of both the first multi-tissue organoid and the second multi-tissue organoid or for the transcriptome of both the first multi-tissue organoid and the second multi-tissue organoid; and
(f) comparing the data of the first multi-tissue organoid to the data of the second multi-tissue organoid.

2. The method of aspect 1, wherein the first PSC or second PSC is an induced PSC (iPSC).

3. The method of aspect 1, wherein the first PSC and second PSC are each an induced PSC (iPSC).

4. The method of any one of aspects 1-3, wherein the data identifies association of a genetic variant with a molecular phenotype.

5. The method of any one of aspects 1-4, wherein the data identifies an expression quantitative trait loci (eQTL).

6. The method of any one of aspects 1-5, wherein the single cell analysis is single cell RNA-sequencing (scRNA-seq).

7. The method of any one of aspects 1-5, wherein the single cell analysis is single cell assay for transposase accessible chromatin (scATAC-seq).

8. The method of any one of aspects 1-7, wherein the mammals are each primates.

9. The method of aspect 8, wherein the primates are each human.

10. The method of any one of aspects 1-9, wherein the method further comprises:
(i) forming a third multi-tissue organoid from a third PSC from a third mammal, wherein the third mammal is of the same species as the first mammal and the second mammal or of a different species than the first mammal and the second mammal;
(ii) permitting the third multi-tissue organoid to differentiate;
(iii) identifying cells within the third multi-tissue organoid by
   (1) clustering cells and annotating the cells based on the genes that are highly expressed in each cluster,
   (2) annotating cell type by correlation of gene expression using a reference data set of known primary cell types, or annotating cell state by correlation of gene expression using a reference data set of genes involved in known cells states, and
   (3) applying topic modeling, matrix factorization, or grades of membership modelling to the annotated cells;
(iv) performing single cell analysis on the third multi-tissue organoid to generate data for the genome or transcriptome of the third multi-tissue organoid,
   the generated data being for the genome of each of the first multi-tissue organoid, the second multi-tissue organoid, and the third multi-tissue organoid, or for the transcriptome of each of the first multi-tissue organoid, the second multi-tissue organoid, and the third multi-tissue organoid; and
(v) comparing the data of the third multi-tissue organoid to the data of the first multi-tissue organoid and to the data of the second multi-tissue organoid.

11. The method of any one of aspects 1-10, wherein identifying cells comprises applying topic modeling.

12. A method of classifying mammals, the method comprising:
(A) analyzing the genome or transcriptome of two or more mammals according to any one of aspects 1-11, wherein the analysis identifies a response expression quantitative trait loci (response eQTL); and
(B) classifying the two or more mammals based on the response eQTL.

13. A method of analyzing a disease-state of a mammal, the method comprising analyzing a genome or transcriptome of a mammal according to the method of any one of aspects 1-11, wherein the genome or transcriptome is associated with a disease-state.

14. The method of any one of aspects 1-13, wherein the data generated is for the genome of each of the multi-tissue organoids.

15. The method of any one of aspects 1-13, wherein the data generated is for the transcriptome of each of the multi-tissue organoids.

16. A method of analyzing a cellular response to a treatment, the method comprising:
(a) forming a first multi-tissue organoid from a first pluripotent stem cell (PSC) from a first mammal;
(b) forming a second multi-tissue organoid from a second PSC from the first mammal;
(c) permitting each of the first multi-tissue organoid and the second multi-tissue organoid to differentiate;
(d) identifying cells within the first multi-tissue organoid and the second multi-tissue organoid by
   (1) clustering cells and annotating the cells based on the genes that are highly expressed in each cluster,
   (2) annotating cell type by correlation of gene expression using a reference data set of known primary cell types, or annotating cell state by correlation of gene expression using a reference data set of genes involved in known cells states, and
   (3) applying topic modeling, matrix factorization, or grades of membership modelling to the annotated cells;
(e) treating the first multi-tissue organoid with a substance while not treating the second multi-tissue organoid with the substance; and
(f) comparing the treatment of the first multi-tissue organoid to the untreated second multi-tissue organoid.

17. The method of aspect 16, wherein the first PSC or second PSC is an induced PSC (iPSC).

18. The method of aspect 16, wherein the first PSC and second PSC are each an induced PSC (iPSC).

19. The method of any one of aspects 16-18, wherein identifying cells comprises applying topic modeling.

20. The method of any one of aspects 16-19, further comprising
(i) forming a third multi-tissue organoid from a third pluripotent stem cell (PSC) from a second mammal, wherein the second mammal is of the same species as the first mammal or of a different species than the first mammal;
(ii) forming a fourth multi-tissue organoid from a fourth PSC from the second mammal;
(iii) permitting each of the third multi-tissue organoid and the fourth multi-tissue organoid to differentiate;
(iv) identifying cells within the third multi-tissue organoid and the fourth multi-tissue organoid by
   (1) clustering cells and annotating the cells based on the genes that are highly expressed in each cluster,
   (2) annotating cell type by correlation of gene expression using a reference data set of known primary cell types, or annotating cell state by correlation of gene expression using a reference data set of genes involved in known cells states, and
   (3) applying topic modeling, matrix factorization, or grades of membership modelling to the annotated cells;
(v) treating the third multi-tissue organoid with a substance while not treating the fourth multi-tissue organoid with the substance; and
(vi) (vi) comparing the treatment of the third multi-tissue organoid to the untreated fourth multi-tissue organoid.

21. The method of aspect 20, further comprising comparing (1) the treatment of the first multi-tissue organoid compared to the untreated second multi-tissue organoid to (2) the treatment of the third multi-tissue organoid compared to the untreated fourth multi-tissue organoid.

22. A method of determining an effect of a substance, the method comprising:
(a) forming two or more multi-tissue organoids, each organoid formed from a separate pluripotent stem cell (PSC) from a mammal;
(b) permitting each of the multi-tissue organoids to differentiate;
(c) identifying cells within each of the multi-tissue organoids by
   (1) clustering cells and annotating the cells based on the genes that are highly expressed in each cluster,
   (2) annotating cell type by correlation of gene expression using a reference data set of known primary cell types, or annotating cell state by correlation of gene expression using a reference data set of genes involved in known cells states, and
   (3) applying topic modeling, matrix factorization, or grades of membership modelling to the annotated cells;
(d) treating the two or more multi-tissue organoids with an amount of the substance, wherein each of the multi-tissue organoids is treated with a different amount of the substance;
(e) comparing the two or more multi-tissue organoids; and
(f) determining an effect of the substance on the two or more multi-tissue organoids.

23. The method of aspect 22, wherein the first PSC or second PSC is an induced PSC (iPSC).

24. The method of aspect 22, wherein the first PSC and second PSC are each an induced PSC (iPSC).

25. The method of any one of aspects 22-24, further comprising:
(i) forming an additional multi-tissue organoid from an additional PSC from the mammal;
(ii) permitting the additional multi-tissue organoid to differentiate;
(iii) identifying cells within the additional multi-tissue organoid by
   (1) clustering cells and annotating the cells based on the genes that are highly expressed in each cluster,
   (2) annotating cell type by correlation of gene expression using a reference data set of known primary cell types, or annotating cell state by correlation of gene expression using a reference data set of genes involved in known cells states, and
   (3) applying topic modeling, matrix factorization, or grades of membership modelling to the annotated cells;
(iv) leaving the additional multi-tissue organoid untreated with the substance; and
(v) comparing the two or more multi-tissue organoids to the additional multi-tissue organoid.

26. The method of any one of aspects 22-25, wherein comparing the two or more multi-tissue organoids to the additional multi-tissue organoid comprises assessing in at least one of the multi-tissue organoids cell death of one or more cell types.

27. The method of aspect 26, wherein the assessment is of all cell types within the multi-tissue organoids.

28. The method of aspect 26 or 27, wherein the assessment comprises using microscopy, live/dead cell staining, immunostaining, or cell counting.

29. The method of any one of aspects 26-28, wherein the assessment comprises using flow cytometry, qPCR, or single cell sequencing.

30. The method of any one of aspects 26-29, wherein the assessment comprises assessing expression of genes associated with cell stress or apoptosis.

31. The method of aspect 30, wherein assessing expression of genes associated with cell stress or apoptosis comprises using low-depth single cell sequencing or qPCR.

32. The method of any one of aspects 26-31, wherein the assessment comprises assessing on-target therapeutic effects on gene expression.

33. The method of aspect 32, wherein assessing on-target therapeutic effects on gene expression comprises using low-depth single cell sequencing or qPCR.

34. The method of any one of aspects 26-33, further comprising determining an optimal dosage, wherein the optimal dosage is the amount of the substance that is determined to cause reduced cell death and reduced stress response across all cell types and to cause on-target effects for a subset of cell types compared to other amounts of the substance.

35. The method of any one of aspects 22-31, wherein the substance is an environmental compound.

36. The method of any one of aspects 22-35, wherein identifying cells comprises applying topic modeling.

37. A method of treating a multi-tissue organoid, the method comprising:
(A) determining an optimal dosage of a substance according to aspect 34;
(B) forming another multi-tissue organoid from a PSC from a mammal;
(C) permitting the multi-tissue organoid of (B) to differentiate; and
(D) treating the differentiated multi-tissue organoid of (C) with the dosage determined in (A).

38. A method of treating a mammal, the method comprising:
(A) determining an optimal dosage of a substance according to aspect 34; and
(B) treating the mammal with the optimal dosage of the substance as determined in (A).

39. The method of aspect 38, further comprising assessing in the treated mammal the health of a cell type that was adversely affected by the substance in a multi-tissue organoid during determination of the optimal dosage.

40. The method of any one of aspects 37-39, wherein identifying cells comprises applying topic modeling.

41. A method of analyzing a cellular response to a treatment, the method comprising:
(a) forming a first multi-tissue organoid from a first pluripotent stem cell (PSC) from a first mammal, wherein the first mammal has a disease-state;
(b) forming a second multi-tissue organoid from a second PSC from a second mammal, wherein the second mammal is of the same species as the first mammal or of a different species than the first mammal and the second mammal does not have the disease-state;
(c) permitting each of the first multi-tissue organoid and the second multi-tissue organoid to differentiate;
(d) identifying cells within the first multi-tissue organoid and the second multi-tissue organoid by
   (1) clustering cells and annotating the cells based on the genes that are highly expressed in each cluster,
   (2) annotating cell type by correlation of gene expression using a reference data set of known primary cell types, or annotating cell state by correlation of gene expression using a reference data set of genes involved in known cells states, and
   (3) applying topic modeling, matrix factorization, or grades of membership modelling to the annotated cells;
(e) treating the first multi-tissue organoid and the second multi-tissue organoid with a substance; and
(f) comparing the treatment of the first multi-tissue organoid to the treatment of the second multi-tissue organoid.

42. The method of aspect 41, wherein the first PSC or second PSC is an induced PSC (iPSC).

43. The method of aspect 41, wherein the first PSC and second PSC are each an induced PSC (iPSC).

44. The method of any one of aspects 41-43, further comprising:
(i) forming an additional multi-tissue organoid from an additional PSC from the first mammal or the second mammal;
(ii) permitting the additional multi-tissue organoid to differentiate;
(iii) identifying cells within the additional multi-tissue organoid by
   (1) clustering cells and annotating the cells based on the genes that are highly expressed in each cluster,
   (2) annotating cell type by correlation of gene expression using a reference data set of known primary cell types, or annotating cell state by correlation of gene expression using a reference data set of genes involved in known cells states, and
   (3) applying topic modeling, matrix factorization, or grades of membership modelling to the annotated cells; and
(iv) leaving the additional multi-tissue organoid untreated with the substance; and
(v) comparing the treatment of the first multi-tissue organoid and the treatment of the second multi-tissue organoid to the additional multi-tissue organoid.

45. The method of any one of aspects 41-44, wherein comparing the treatment of the organoids comprises assessing in at least one of the multi-tissue organoids cell death of one or more cell types.

46. The method of aspect 45, wherein the assessment is of all cell types within the multi-tissue organoids.

47. The method of aspect 45 or 46, wherein the assessment comprises using microscopy, live/dead cell staining, immunostaining, or cell counting.

48. The method of any one of aspects 45-47, wherein the assessment comprises using flow cytometry, qPCR, or single cell sequencing.

49. The method of any one of aspects 45-48, wherein the assessment comprises assessing expression of genes associated with cell stress or apoptosis.

50. The method of aspect 49, wherein assessing expression of genes associated with cell stress or apoptosis comprises using low-depth single cell sequencing or qPCR.

51. The method of any one of aspects 45-50, wherein the assessment comprises assessing on-target therapeutic effects on gene expression.

52. The method of aspect 51, wherein assessing on-target therapeutic effects on gene expression comprises using low-depth single cell sequencing or qPCR.

53. The method of any one of aspects 41-52, wherein identifying cells comprises applying topic modeling.

It shall be noted that the preceding are merely examples of aspects of the disclosure. Other exemplary aspects are apparent from the entirety of the description herein. It will also be understood by one of ordinary skill in the art that each of these aspects may be used in various combinations with the other aspects provided herein.

The following examples further illustrate aspects of the disclosure, but, of course, should not be construed as in any way limiting its scope.

### EXAMPLE

### Materials and Methods

### Samples

iPSC lines from eight unrelated individuals from the Yoruba HapMap population were used to form MTOs. The iPSC lines were reprogrammed from lymphoblastoid cell lines (LCLs) and were characterized and validated previously (Banovich et al., 2018). The original LCL lines were genotyped by the HapMap project and identity of the stocks used in this study is confirmed by scRNA-seq data collected for this study (Belmont et al., 2003). All cell lines used in this study tested negative for mycoplasm. Lines 18511, 18858, 18912, 19140, and 19159 are from female individuals. Lines 19160, 18856, and 19210 are from male individuals. Preprocessing and analysis of lines 18511, 18858, and 19160 are described throughout the Materials and methods section.

iPSC Maintenance

Feeder-free iPSC cultures were maintained on Matrigel Growth Factor Reduced Matrix (CB-40230, Thermo Fisher Scientific) with StemFlex Medium (A3349401, Thermo Fisher Scientific) and Penicillin/Streptomycin (30,002 Cl, Corning). Cells grew in an incubator at 37° C., 5% CO2, and atmospheric O2. Every 3-5 days thereafter, the cells were passaged to a new dish using a dissociation reagent (0.5 mM EDTA, 300 mM NaCl in PBS) and seeded with ROCK inhibitor Y-27632 (ab120129, Abcam).

### MTO Formation and Maintenance

MTOs were formed using a modified version of the STEMCELL Aggrewell 400 protocol. Briefly, wells of an Aggrewell 400 24-well plate (34415, STEMCELL) were coated with anti-adherence rinsing solution (07010, STEMCELL). iPSCs were dissociated and seeded them into the Aggrewell 400 24-well plate at a density of 1,000 cells per microwell (1.2×106 cells per well) in Aggrewell MTO Formation Medium (05893, STEMCELL). After 24 hr, half of the spent media was replaced with fresh Aggrewell MTO Formation Medium. Forty-eight hr after seeding the Aggrewell plate, MTOs were harvested and moved to an ultra-low attachment six-well plate (CLS3471-24EA, Sigma) in E6 media (A1516401, ThermoFisher Scientific). MTOs were maintained in culture for an additional 19 days, replacing media with fresh E6 every 48 hr. Three replicates were performed of MTO formation on different days; each replicate included all three lines.

### MTO Dissociation

MTOs 21 days after formation were collected and dissociated. MTOs were dissociated by washing them with phosphate-buffered saline (PBS) (Corning 21-040-CV), treating them with AccuMax (STEMCELL 7921) and incubating them at 37° C. in for 15-35 min. After 10 min in Accumax, MTOs were pipetted up and down with a clipped p1000 pipette tip for 30 s. Pipetting was repeated every 5 min until MTOs were completely dissociated. Dissociation was then stopped by adding E6 media and straining cells through a 40 μm strainer (Fisherbrand 22-363-547). Cells were resuspended in PBS and counted them with a TC20 Automated Cell Counter (450102, BioRad). Before scRNA-seq, an equal number of cells from each line was mixed together.

### Single-Cell Sequencing

scRNA-seq data was collected using the 10× Genomics V3.0 kit. Single-cell collections for this experiment were mixed with cells from a larger experiment in all three replicates. From the first replicate of MTO differentiations, MTO cells from YRI individuals 18511, 18858, and 19160 were mixed with MTO cells from an additional three humans and chimpanzees (nine individuals total). Even numbers of cells from all nine individuals were collected across nine lanes of a 10×chip, targeting 10,000 cells per lane. In this replicate, reagents from three different 10×kits were used. From replicates 2 and 3 of MTO differentiation, MTOs were only generated from the same three YRI individuals (18511, 18858, and 19160) and the three chimpanzees (six individuals total). In each replicate, even numbers of cells of each individual were mixed and cells were collected in four lanes of a 10× chip, targeting 10,000 cells per lane, and samples were processed using reagents from a single 10× kit.

Libraries were sequenced using paired-end 100 base pair sequencing on the HiSeq 4000 in the University of Chicago Functional Genomics Core. For libraries from replicate 1, equal proportions of each of the six MTO libraries were mixed and the pooled samples were sequenced on one lane of the HiSeq 4000. Preliminary analyses showed that two of these lines were low quality. One of the low-quality libraries was remade and the other was discarded. Then equal proportions of the remade library were mixed with the remaining three libraries from replicate one and sequenced the pooled samples on one lane of the HiSeq 4000. Preliminary analyses indicated that three out of four of these libraries were below optimal quality, but would produce usable data. Then samples from the final eight libraries from replicate 1 were pooled together, mixing equal parts of each of the five high-quality libraries with half the amount of the other three, and this pool was deep-sequenced on eight lanes of the HiSeq 4000. For replicate two libraries, equal parts of all four libraries were mixed and sequenced on one lane. After confirming that each library was high-quality, the same pool was deep-sequenced on six additional lanes of the HiSeq 4000. For replicate three libraries, equal parts of all four libraries were mixed and sequenced on one lane. After confirming that each library was high-quality, the same pool was deep-sequenced on four additional lanes of the HiSeq 4000. In all cases, the number of lanes for deep sequencing was calculated to reach 50% saturation.

### Alignment and Sample Deconvolution

STARsolo was used to align samples to both the human genome (GRCh38) (Dobin et al., 2013) and the chimpanzee genome (Clint PTRv2/panTro6; retrieved from GenBank January 2018). Gene annotations from ensemb198 and transmapV5 were used, respectively. In order to separate human cells from chimpanzee cells, discordant reads—those that mapped with different scores in each species, were identified. A cell was identified as human if (1) at least five discordant reads that had a higher human mapping score and (2) at least 80% of discordant reads had a higher human mapping score. The remainder of analyses in this work were restricted to these human cells. Individual samples were demultiplexed and doublets were identified using demuxlet (Kang et al., 2018). For this demultiplexing with demuxlet, previously collected and imputed genotype data for these three Yoruba individuals from the HapMap and 1000 Genomes Project was used (Auton et al., 2015; Belmont et al., 2003).

### Filtering and Integration

EmptyDrops were run to identify barcodes tagging empty droplets and only barcodes with a high probability of tagging a cell-containing droplet were kept (i.e. cells with an EmptyDrops FDR<0.0001 were kept) (Lun et al., 2019). Cells labeled as doublets or ambiguous by demuxlet were removed, keeping only barcodes labeled as singlets. The data was also filtered to include only high-quality cells expressing between 3% and 20% mitochondrial reads and expressing more than 1500 genes. Data from each 10× lane were normalized using SCTransform in Seurat (Butler et al., 2018; Hafemeister and Satija, 2019). In total, 42,488 high-quality cells were obtained. Then data from each of the 10× lanes from all replicates were merged, the data were scaled, and principal components analysis (PCA) was run using 5000 variable features. Then data were integrated with Harmony to correct the PCA embeddings for batch effects and individual effects (Korsunsky et al., 2019).

### Clustering and Cell Type Annotation

To cluster the data, Seurat's FindNeighbors were applied using 100 dimensions from the Harmony-corrected reduced dimensions, followed by FindClusters at resolutions 0.1, 0.5, 0.8, and 1.

Differential expression analysis was performed using the limma R package (Ritchie et al., 2015). First, genes were filtered to include only those expressed in at least 20% of cells in at least one cluster at a given clustering resolution. Then pseudobulk expression values were calculated for each individual-replicate-cluster grouping (i.e. cells from the same individual, same replicate, and same cluster assignment). Accordingly, pseudobulk expression values were defined as the sum of normalized counts in each group. Next pseudobulk expression values were TMM-normalized and voom was used to calculate a weighted gene expression value to account for the mean-variance relationship (Law et al., 2014). Then the following linear model was fit:

Y=0+β_{cluster}*x+β_{replicate}*x+β_{individual}*x

Contrasts were used to first test for differential expression of each cluster compared to all other and then to test for differential expression between pairs of similar clusters to find distinguishing markers. Cell type identity of each cluster was annotated based on significant differential expression of the known marker genes.

### Assessment of Cell Type Composition and Differentiation Efficiency in Five Additional Lines

To evaluate the cell type composition resulting from MTO differentiation of YRI iPSC lines more generally, five additional randomly chosen lines (18856, 18912, 19140, 19159, and 19210) from the YRI iPSC panel were differentiated. iPSCs were differentiated and dissociated in parallel using the same protocols described above. After dissociation, cells from each individual in equal proportions were mixed and scRNA-seq data was collected using the 10× genomics V3.1 kit, targeting collection of 10,000 cells per lane and 10,000 cells per individual. Notably, cells from these five lines were mixed with cells from two additional lines (each with distinct genotypes) from a separate experiment during 10× collections. Libraries were sequenced using paired-end 100 bp sequencing on the NovaSeq 6000 at the University of Chicago Genomics Core. Samples were aligned to the human genome (GRCh38) using CellRanger (Zheng et al., 2017). Cells were then assigned to individuals. Demuxlet was used to identify doublets with previously collected and imputed genotype data for the five additional YRI individuals; this data originated from the HapMap and 1000 Genomes Projects (Kang et al., 2018; Auton et al., 2015; Belmont et al., 2003). Finally, cells assigned to individuals that were not a part of this experiment were removed. Doublets were filtered out, as well as cells with greater than 15% mitochondrial reads or fewer than 3% mitochondrial reads, and cells with fewer than 1000 unique genes expressed.

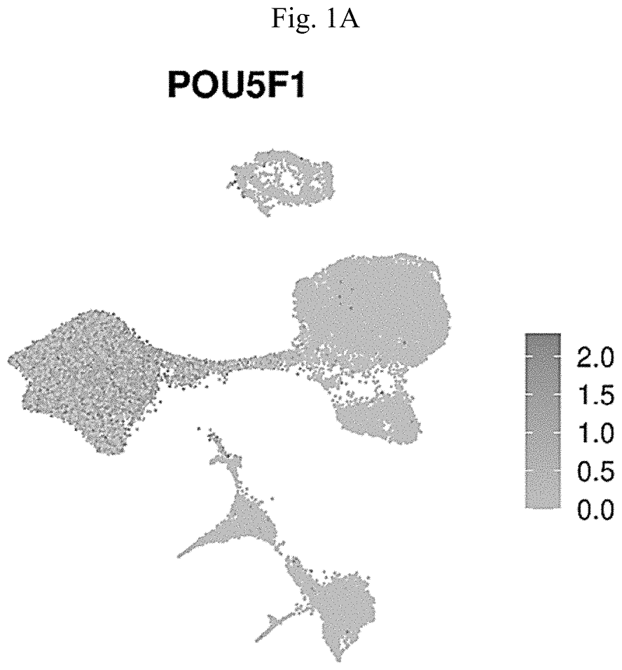

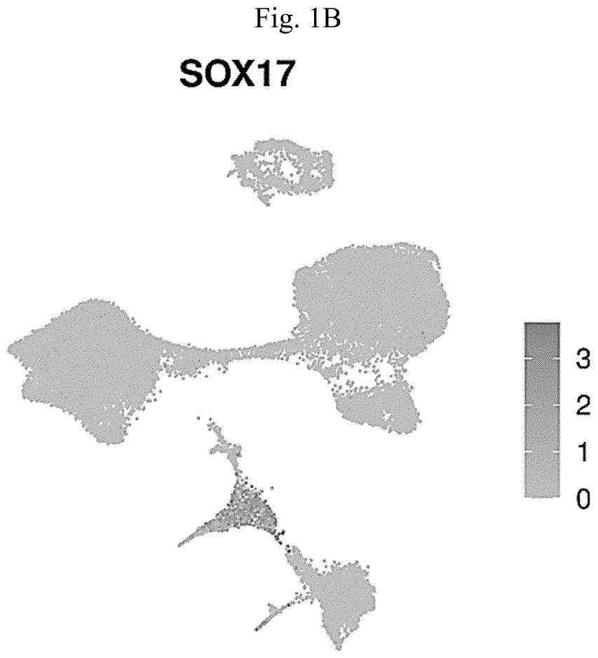

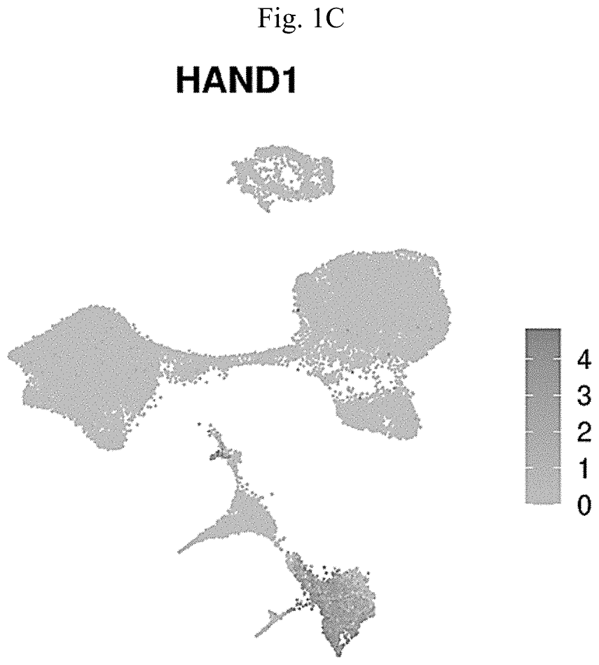

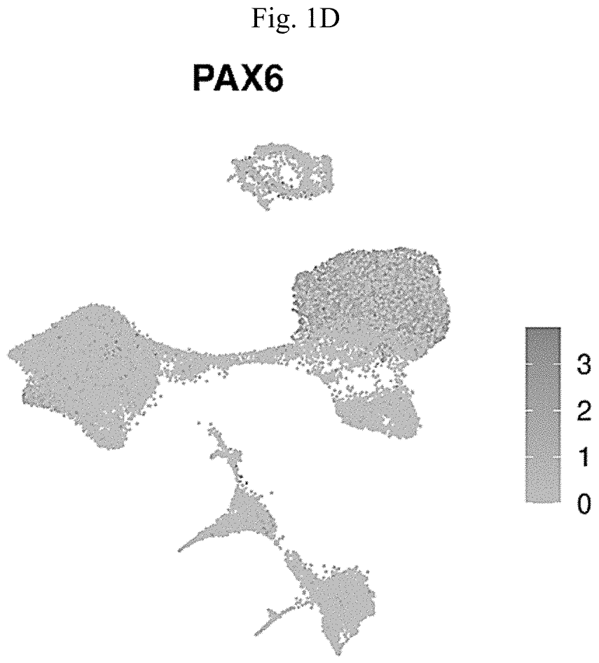

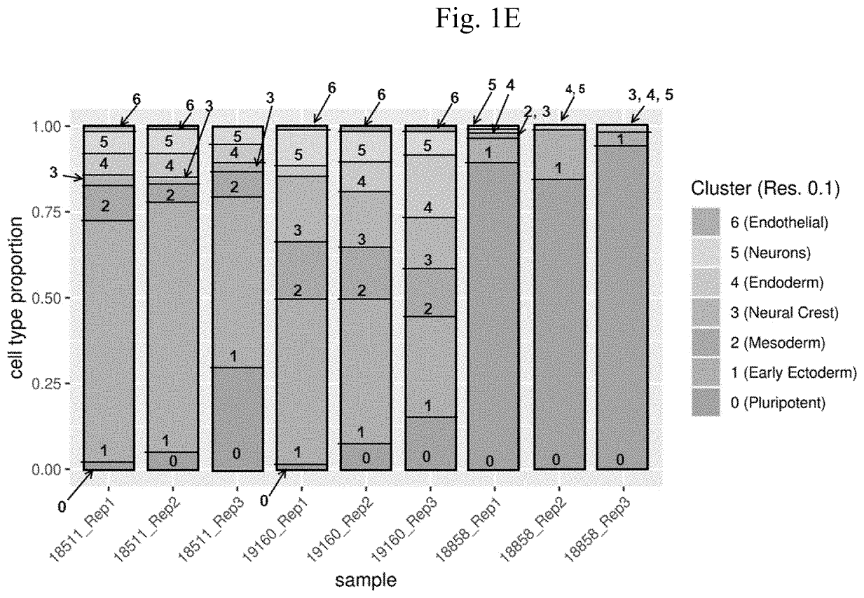

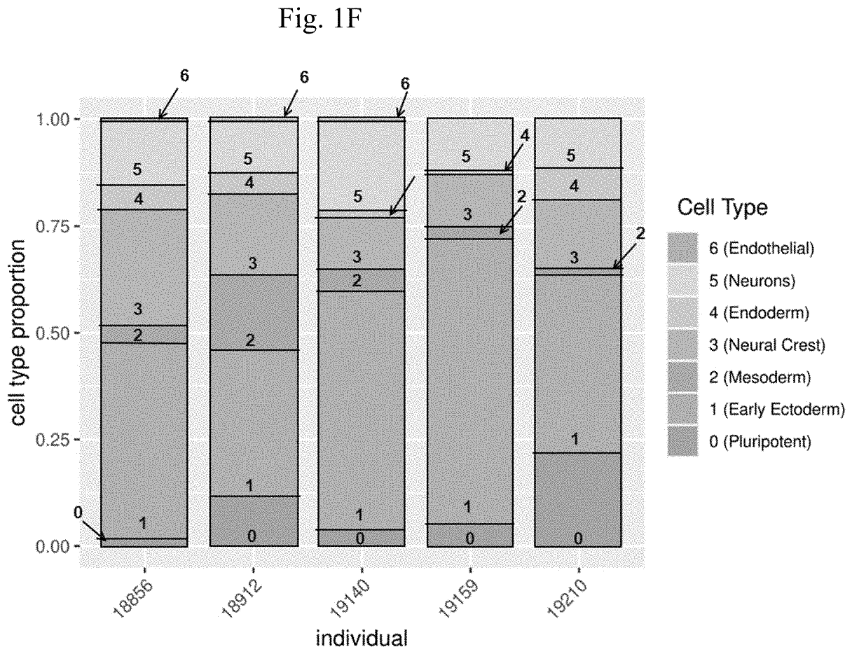

Data was then normalized using SCTransform (Butler et al., 2018; Hafemeister and Satija, 2019), clusters were identified using the Louvain algorithm in Seurat (at Resolution 0.15), and expression was visualized for canonical marker genes and the most significant marker genes of clusters identified in differential expression analysis (FIG. 1—figure supplement 4). Based on marker gene expression, clusters 0 and 2 represent early ectoderm, cluster one represents neural crest cells, cluster three represents pluripotent cells, clusters 4 and 6 represent neurons, cluster 5 represents mesoderm, cluster seven represents endoderm, and cluster 8 represents endothelial cells. The proportion of cells from each individual that were assigned to each of these cell type categories was calculated. Each of these five additional cell lines exhibits high differentiation efficiency, comparable to that of iPSC lines 18511 and 19160. Additional lines were also integrated with reference data to annotate cell types as described below.

### Reference Integration and Label Transfer

Next cells were compared to reference data sets of primary fetal cell types, Day 20 hESC-derived MTOs, and hESCs (Cao et al., 2020; Han et al., 2020). To integrate cells with the reference sets, first each reference set was subset to include only protein coding genes. Because the Cao et al. reference set is so large, containing over 4 million cells, cells from this reference set were subset to include a maximum of 500 cells per cell type. Each reference set was then normalized using SCTransform (Butler et al., 2018; Hafemeister and Satija, 2019). Then the data sets were merged using Seurat, SCTransform was rerun, regressing out data set specific effects of sequencing depth, the data were scaled, and PCA was ran. Then Harmony was run to correct PCA embeddings for the effects of each data set to complete the integration (Korsunsky et al., 2019). Cell type annotations were then transferred from cell types present in the fetal reference and hESC to MTO cells. For each MTO cell, the five nearest reference cells were found in Harmony-corrected PCA space based on Euclidean distance; if at least ⅗ of the nearest reference cells shared an annotation, that annotation was transferred to the MTO cell. If fewer than three of the nearest reference cells shared an annotation, the MTO cell was annotated as ‘uncertain’.

To assess the quality of the reference integration strategy, testing was performed to determine whether (1) datasets are being over-corrected and (2) MTO cells annotated using reference cell types express expected marker genes. MTO cells were first subsetted to broad cell type categories identified using clustering (at resolution 0.1) and differential expression analysis: Pluripotent (cluster 0), Early Ectoderm (cluster 1), Endoderm (cluster 4), Meso-derm (clusters 2, 6), Neural Crest (cluster 3), and Neurons (cluster 5). Using each subset of cells, the reference integration pipeline was repeated by merging the MTO cells with three reference data sets (fetal cells, hESCs, and an external set of Day 20 MTOs), normalizing using SCTransform, running PCA using 5,000 variable features, integrating the data using Harmony, and transferring labels based on the five nearest reference cells (see Materials and methods) (Butler et al., 2018; Hafemeister and Satija, 2019; Korsunsky et al., 2019). 79% of MTO cells are assigned to the same cell type in the full integration and subset integration. Of MTO cells that are annotated differently in the full and subset integrations, 82% were labeled as ‘hESC’ or ‘uncertain’ in either the full or subset integration. This suggests that differences in these annotations are often be due to slight changes in the positioning of cells between the hESC reference cells and fetal reference cells; this is expected when pluripotent cells are not included in subsets of MTO cells. And, importantly, cells are not often annotated as a different fetal cell type. Together, these results suggest that this integration approach is robust to subsetting input cell types and is likely not over-integrating the test and reference data sets.

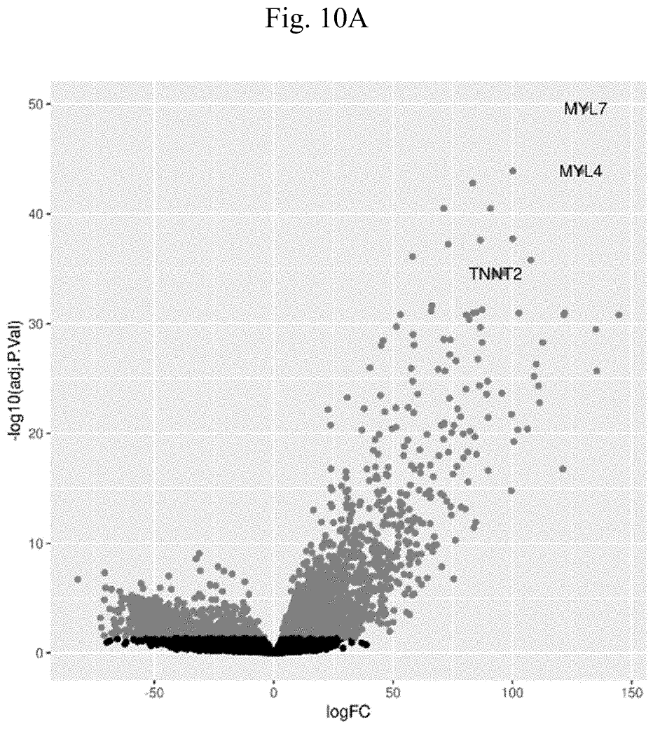

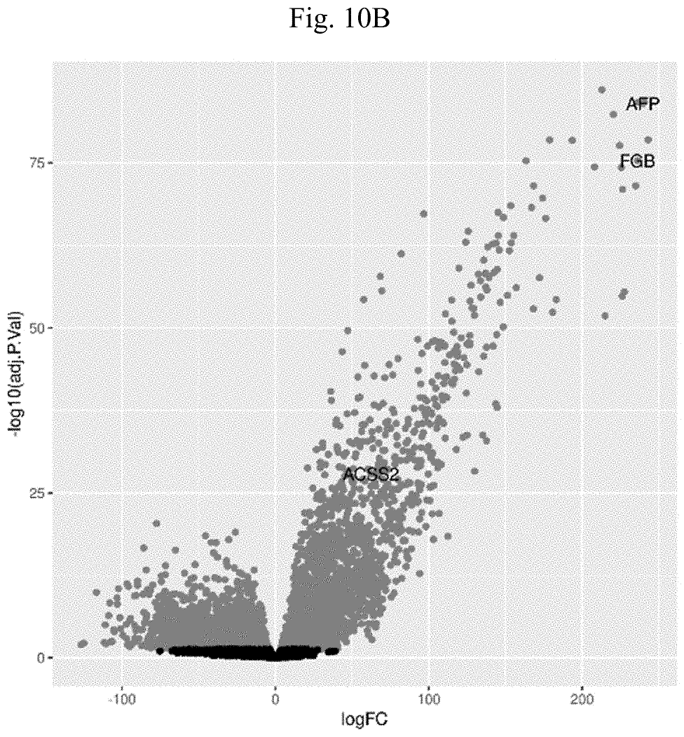

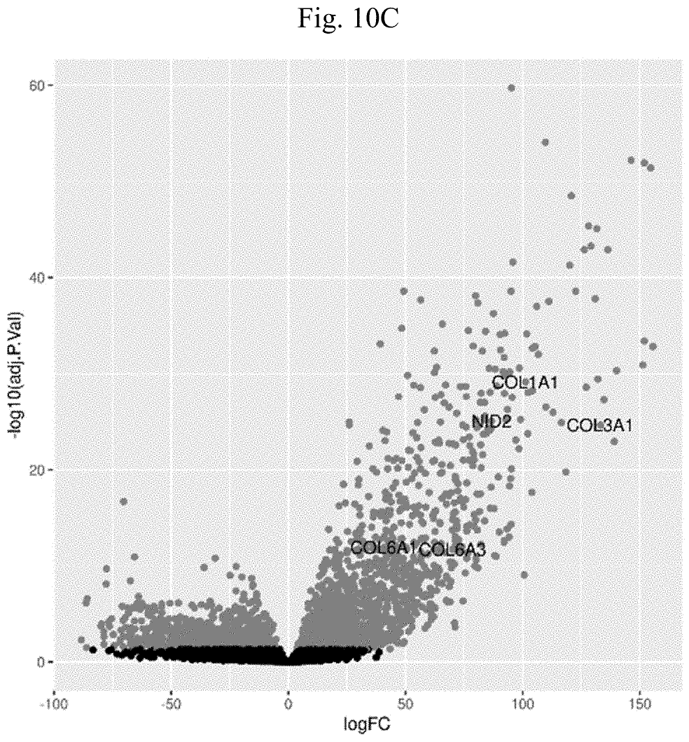

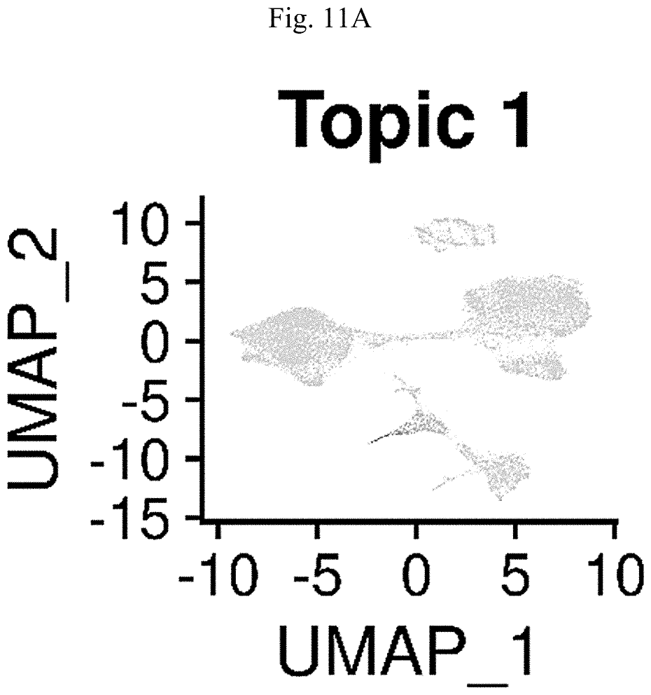

Next, testing was performed to determine whether annotated MTO cells differentially express expected marker genes. This analysis was limited to annotations with at least 10 total MTO cells from at least two individuals in two replicates. Pseudobulk expression was then calculated for cells of the same annotation, individual, and replicate, and genes were filtered to include only those with at least 10 counts in at least one sample and at least 15 total counts across all samples. Pseudobulk expression values were then TMM-normalized, voom was used to calculate a weighted gene expression value, and differential expression between annotations was tested for using limma. Of the annotations tested, the most significantly differentially expressed genes often included known cell type markers. For example, cells annotated as cardiomyocytes showed significant upregulation of of MYL7, MYL4, and TNNT2 (FIGS. 10A, 10B, and 10C). Cells annotated as hepatoblasts showed significant upregulation of AFP, FGB, and ACSS2. Cells annotated as mesothelial cells showed significant upregulation of NID2 and collagen genes (COL6A3, COL1A1, COL3A1, COL6A1). These results provide further support that this reference integration approach yields meaningful annotation of MTO cells.

### Topic Modeling

Topic modeling was also performed using FastTopics to learn major patterns in gene expression within the data set, or topics, and to model each cell as a combination of these topics. For this analysis, raw counts were used and the data was filtered to include genes expressed in at least 10 cells. A Poisson non-negative matrix factorization was then pre-fit by running 1000 EM updates without extrapolation to identify a good initialization at values of k equal to 6, 10, 15, 25, and 30. This initialization was used to fit a non-negative matrix factorization using 500 updates of the scd algorithm with extrapolation to identify 6, 10, 15, 25, and 30 topics. FastTopics' diff_count_analysis function was then used to identify genes differentially expressed between topics. These differentially expressed genes were used to interpret the cellular functions and identities captured by each topic. In some cases, differentially expressed genes included known marker genes (Table 1).

TABLE 1
Top 15 driver genes of each topic from the k = 6 topic model based on Z-score.

| Topic | Top 15 driver genes |
|---|---|
| k1 | S100A10, FTL, FN1, APOC1, CST3, APOE, SERPINE2, KRT19, CKB, S100A11, |
| | LGALS3, TMSB10, S100A16, AFP, PTGR1 |
| k2 | MT-CO2, MT-CO3, MT-CO1, MT-CYB, PRDX1, MT-ND4, MT-ATP6, GSTP1, |
| | MT-ND1, RPL8, APOE, RPSA, RPL12, PFN1, HMGA1 |
| k3 | PTMA, NCL, RPL23, SET, HSP90AB1, TPL27A, MT-ND4, L1TD1, SERBP1, TERF1, |
| | HSPD1, CENPF, DPPA4, MT-ATP6, UGP2 |
| k4 | S100A10, KRT19, S100A11, VIM, MDK, TMSB10, KRT8, SPARC, COL1A1, FN1, |
| | COL1A2, COL6A2, KRT18, TPM1, ANXA2 |
| k5 | TUBA1A, VIM, MARCKSL1, MARCKS, TUBA1B, MAP1B, ID3, CRABP1, PTMS, |
| | TMSB10, H1FX, STMN1, CENPV, CRABP2, NUCKS1 |
| k6 | RPS27, VIM, LDHA, GAPDH, IGFBP2, TUBA1A, APOA1, RPL13, TMSB10, S100A10, |
| | RPL6, RPL30, RPL9, RPS19, RPL37 |

### Hierarchical Clustering Based on Cell Type Composition and Gene Expression

To understand how similar cell type composition is between replicates and individuals, first the proportion of cells from each individual in each replicate assigned to each Seurat cluster at resolution 0.1 was calculated. Then, using the ComplexHeatmap R package and performing hierarchical clustering based on the complete linkage method, the clustering of these replicate-individual groups (Gu et al., 2016) was visualized. This analysis was repeated using Seurat clusters at resolution 0.5, 0.8, and 1 to show that the overall patterns of hierarchical clustering are robust to cluster resolution. An analogous analysis was performed using topic loadings instead of cluster proportions. Here, the loading of each topic on cells from the same individual and replicate was determined, then the same hierarchical clustering was used with ComplexHeatmap to visualize patterns of similarity between cells of each individual and replicate (Gu et al., 2016).

Hierarchical clustering was also performed on gene expression of individual cells. To do so, the pseudobulk expression for each individual-replicate-cluster group was taken and filtered to genes expressed in at least 20% of cells in at least one cluster. The log 10 counts per million expression of each gene was then calculated. A heatmap using the ComplexHeatmap R package was then generated, again performing hierarchical clustering based on the complete linkage method (Gu et al., 2016).

### Variance Partitioning

Using the same pseudobulk data and precision weights computed by voom from differential expression analysis, the VariancePartition R package was used to quantify the variation attributable to cluster, replicate, and individual (Hoffman and Schadt, 2016). A random effect model was fit and modeled cluster, replicate, and individual as random effects. This analysis was performed across all tested Seurat clustering resolutions (0.1, 0.5, 0.8, 1). This analysis was performed using both pseudobulk samples of cells from the same cluster, replicate, and individual and at single-cell resolution with each cell as a sample. The variance in each Seurat cluster was partitioned separately using a random effect model with terms for replicate and individual. For this analysis, pseudobulk samples of cells from each individual and replicate were used.

### Power Analysis

To ascertain the power to detect eQTLs and dynamic eQTLs across a range of sample sizes, standardized effect sizes, and experiment sizes a power function was used as derived in Sarkar et al., 2019:

$$P o w ( β , α , n , σ ) = Φ ( {Φ}^{- 1} \frac{α}{2} + \frac{β}{σ} \sqrt{n} )$$

where β denotes the true additive significance level, α denotes the significance level, n denotes sample size, and 6 represents the phenotype standard deviation. This approach assumes a simple linear regression for eQTL mapping and a conservative Bonferroni correction (FWER=0.05, assuming 10,000 genes tested, α=5e-6).

To estimate the standard deviation for a given experiment size, cells were downsampled from this experiment, sampling evenly between individuals and replicates to range of experiment sizes from 2700 cells to 21,600 cells. For each experiment size, 10 random samples of cells were taken and pseudobulk expression of cells was calculated from the same cluster (defined at resolution 1), individual, and replicate. Genes were filtered to include those expressed in at least 20% of cells in at least one cluster (at resolution 1) in the full set of MTO cells. For each sample the median variance attributable to residuals was then partitioned using the variancePartition package. The median of the median variance from the 10 samples at each experiment size was taken and was used to fit an exponential decay model to quantify the relationship between experiment size and residual variance. The square root of this variance was used to determine the standard deviation for a given experiment size in the power calculations.

### Trajectory Inference and Identification of Dynamic Gene Modules

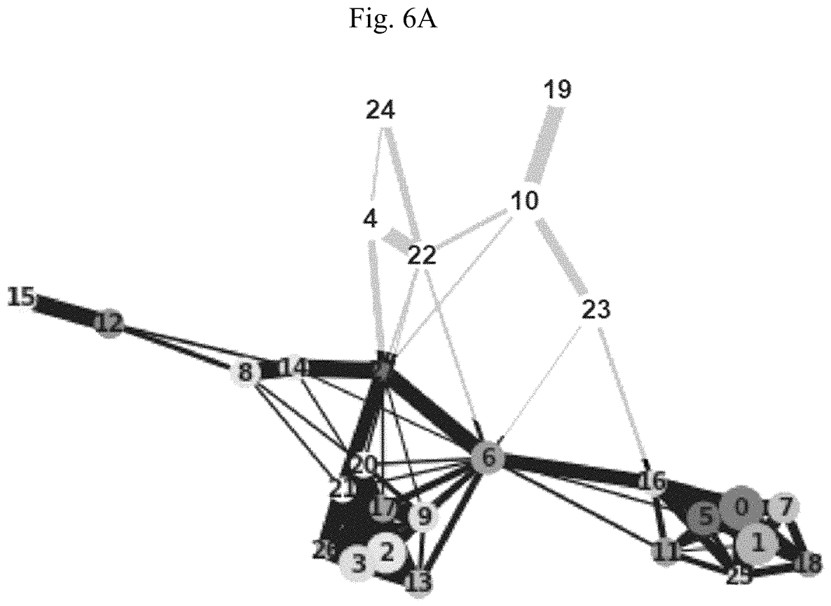

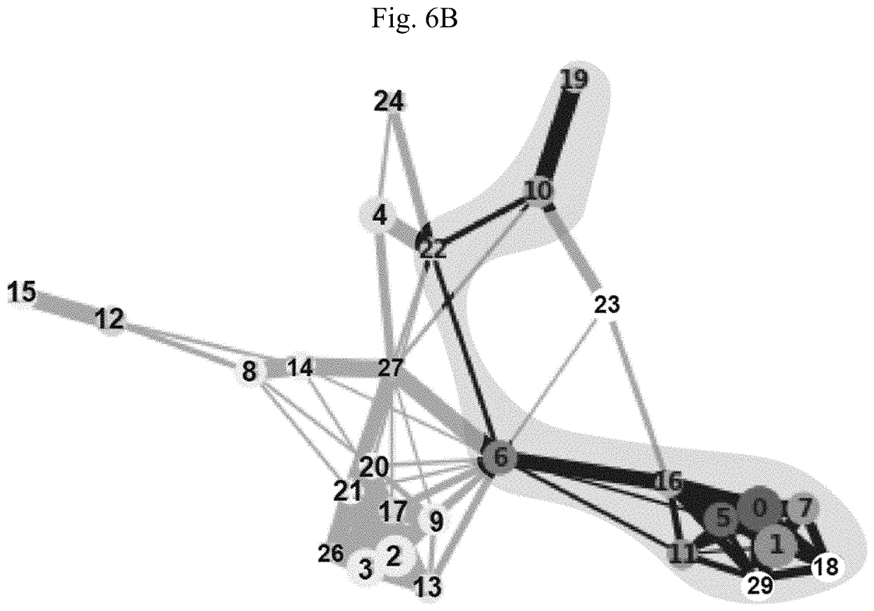

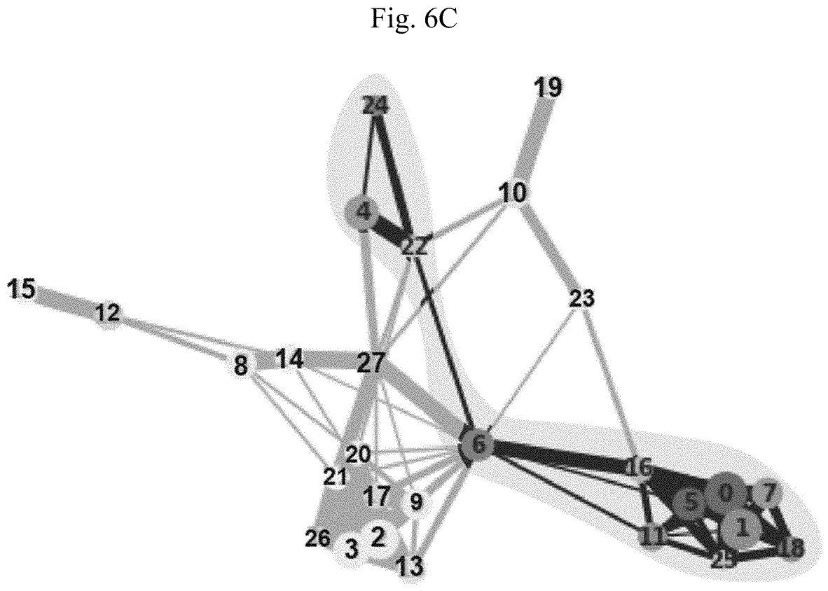

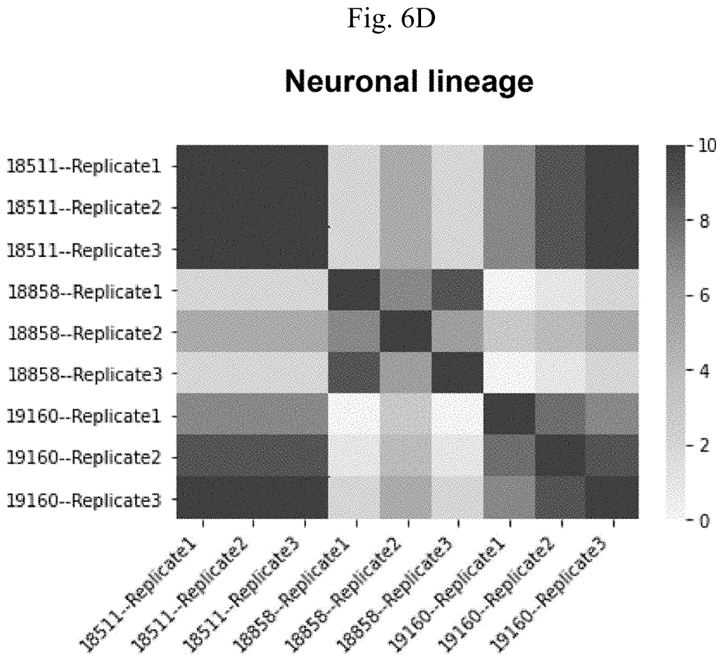

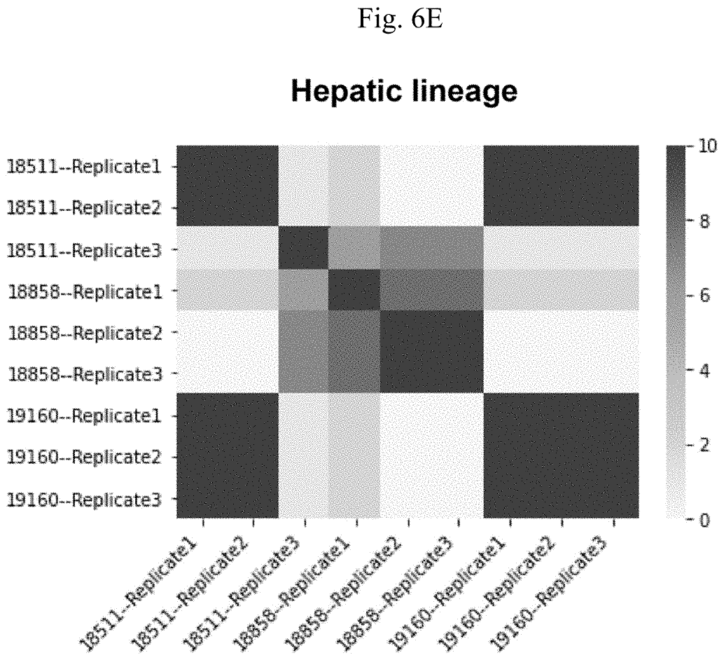

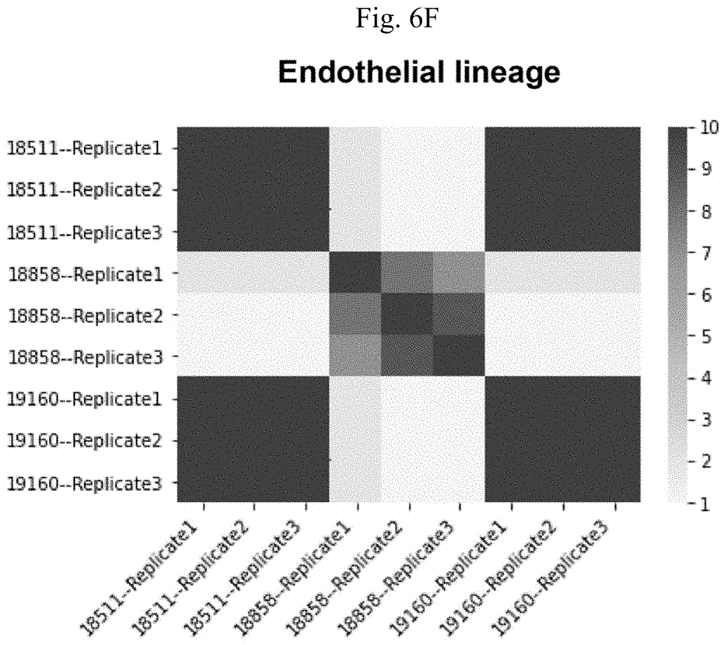

Trajectories were inferred using PAGA in Scanpy using Seurat clusters at all tested resolutions (Wolf et al., 2019). Pseudo-time was assigned using diffusion pseudo-time with the pluripotent cells assigned as the root (Haghverdi et al., 2016). Known developmental trajectories were then traced supported by the PAGA graph. At clustering resolution 1, the trajectory was traced from pluripotent cells to hepatocytes (clusters 0, 1, 5, 6, 7, 10, 11, 16, 18, 19, 25, and 22), pluripotent cells to endothelial cells (clusters 0, 1, 4, 5, 6, 7, 11, 16, 18, 22, 24, and 25), and pluripotent cells to neurons (clusters 0, 1, 2, 3, 5, 6, 7, 8, 9, 11, 12, 13, 14, 15, 16, 17, 18, 20, 21, 26, and 27) (FIGS. 6A, 6B, and 6C).

Cells were then isolated from each of these three trajectories and Split-GPM was used to simultaneously cluster samples and identify dynamic gene modules. For this analysis, data were divided into decile pseudo-time bins and pseudobulk gene expression was calculated for cells of the same individual, replicate, and pseudo-time bin. 20 dynamic gene modules were identified in each trajectory and they were interpreted using gene set enrichment. To understand the variation in dynamic gene expression between individuals and replicates, split-GPM was rerun ten times and how often cells from each individual and replicate were assigned to the same sample cluster was observed.

### Results

### Study Design, Data Collection, and Preprocessing

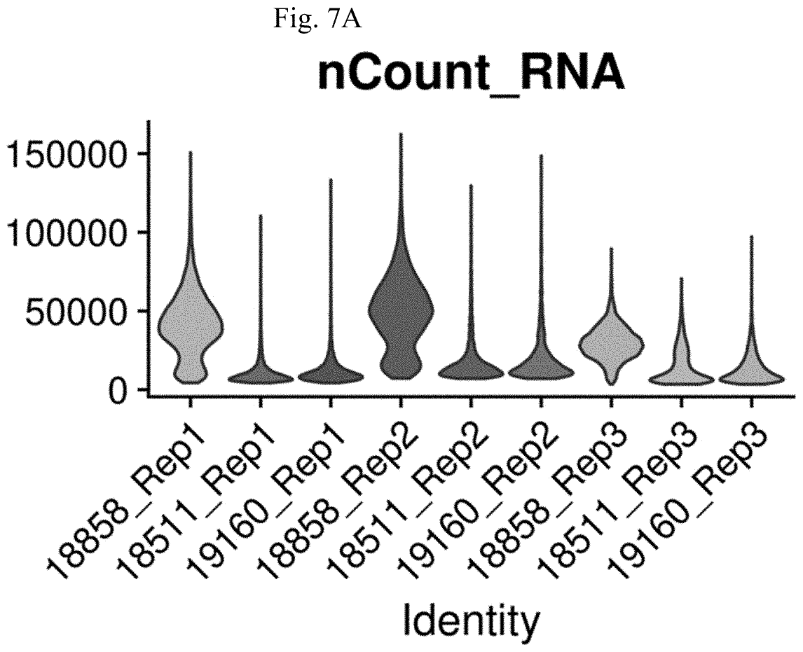

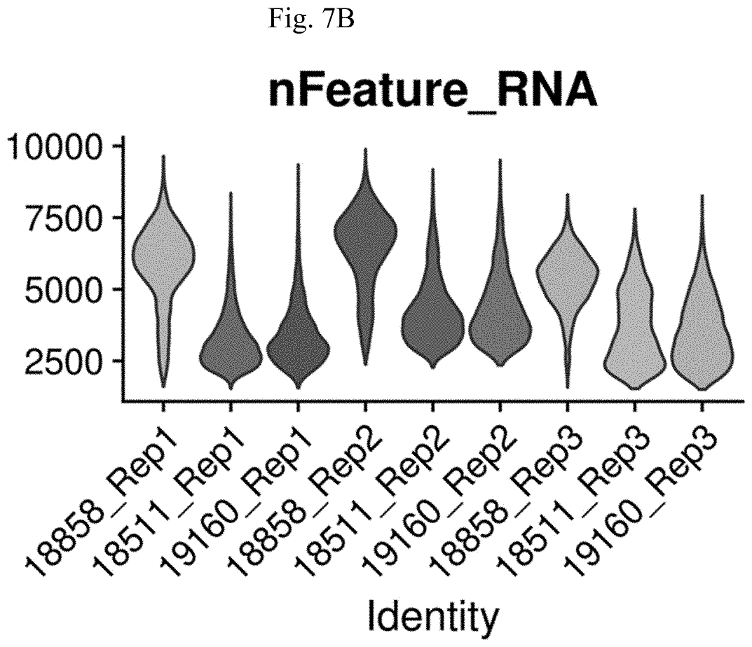

To characterize sources of variation in gene expression in human MTOs, MTOs were initially differentiated from three human iPSC lines (18511, 18858, and 19160) in three replicates. The experiment was performed in three batches, where each batch includes one replicate from each of the three individuals. MTOs differentiate quickly, with cell types representing endoderm, mesoderm, and ectoderm present after 8 days (Han et al., 2018). In this study, MTOs were maintained for 3 weeks after formation, allowing cells to continue to differentiate and mature. After 21 days, scRNA-seq data were collected, targeting equal numbers of cells from each individual in each replicate. After filtering and quality control, high-quality data were retained from a sample of 42,488 cells (an average of 4721 cells per individual/replicate). For these cells, a median of 16,712 UMI counts per cell was obtained, which allowed for measuring the expression of a median of 4274 genes per cell (FIGS. 7A and 7B). Data from all cells were integrated using Harmony, which anchors the data sets by cell type (Korsunsky et al., 2019).

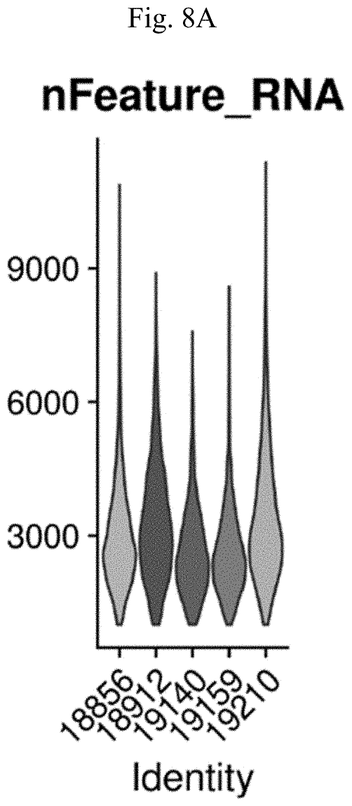

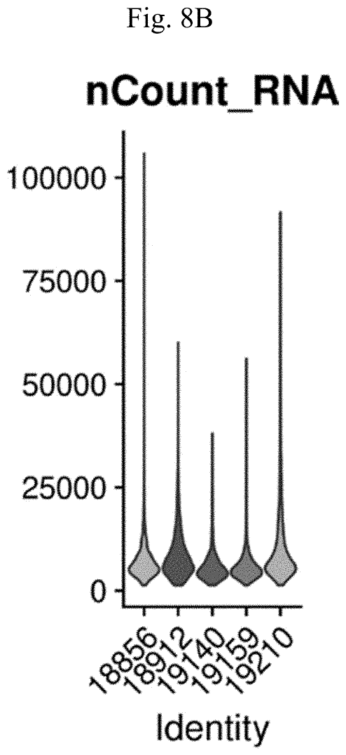

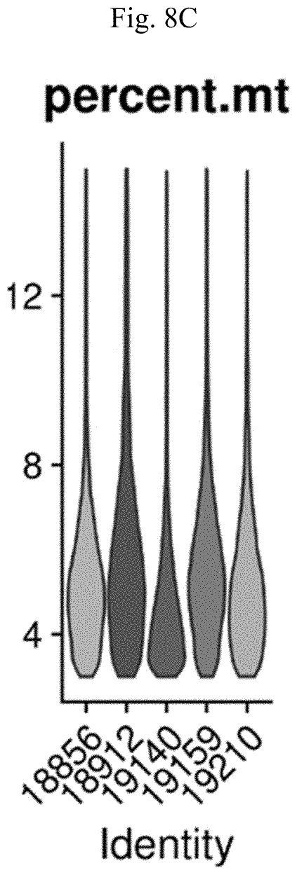

After these initial collections, one individual, 18858, was found to have lower differentiation efficiency than the other two lines (FIG. 1E, see differentiation efficiency across individuals). Too assess the robustness of differentiation efficiency and cell type composition among a larger sample of individuals from the YRI iPSC panel, MTOs were differentiated in one replicate from each of five additional randomly chosen lines (18856, 18912, 19140, 19159, and 19210). After filtering and quality control, an average of 5243 cells per individual were retained in this new set of data, a median of 5983 UMI counts per cell, and a median of 2775 genes per cell (FIGS. 8A, 8B, and 8C). Throughout the text, when data from the five lines that were subsequently collected using only a single replicate (18856, 18912, 19140, 19150, and 19210) were used, those lines are referred to as the ‘additional lines’. The initial replicated data collected from individuals 18511, 18858, and 19160 are used in all analyses throughout this study, while the additional lines are used only to demonstrate consistency of cell type composition across individuals.

To validate the expectation that MTOs should contain cells from each germ layer, the expression of early developmental marker genes was first characterized. Cells were found expressing markers for endoderm (SOX17, FOXA2), mesoderm (HAND1), and ectoderm (PAX6), in addition to cells still expressing pluripotency markers (POU5F1, MYC, NANOG). The data were visualized with uniform manifold approximation and projection (UMAP) and the cells expressing each of these germ layer markers occupied distinct groups in UMAP space (FIGS. 1A, 1B, 1C, and 1D; Becht et al., 2018). Moreover, every replicate in the experiment, regardless of the individual, was found to include cells from all three germ layers (FIG. 1E and Table 2).

Next the heterogeneous cell types present in these MTOs were further explored. In studies of scRNA-seq data from tissues and samples with well-characterized cell type composition, clustering is often applied to demarcate populations of pure cell types within heterogeneous samples. In these studies, clustering resolution, which determines the number of clusters identified by the algorithm, is typically chosen to recapitulate the expected number of known cell types. The identified clusters can be annotated based on the expression of known marker genes.

However, this experiment there was no a priori knowledge of the exact number or types of cells that would result from the spontaneous differentiation of the MTOs. Hence, three complementary approaches were used to annotate cells, capturing various perspectives on what might define a cell type in this data set. First, cell types were identified by clustering cells and annotating the cells based on the genes that are highly expressed in each cluster. Second, cell types were annotated by considering the correlation of gene expression in the data with a reference data set of known primary cell types. For the third approach, a different perspective was used, and topic modeling was applied to consider a less discrete definition of cell type.

For the first approach, a standard clustering analysis was used, the Louvain algorithm in Seurat, to identify groups of cells with similar transcriptomes (Blondel et al., 2008). To avoid making assumptions about the true number of cell types present, this analysis was repeated across different clustering resolutions (resolution 0.1, 0.5, 0.8, and 1). As expected, the number of clusters identified varied greatly depending on the resolution (Table 2). Each subsequent analysis was performed using clusters defined at multiple resolutions, to ensure that the qualitative conclusions are robust with respect to the number of clusters identified.

For each clustering resolution, pseudobulk gene expression levels were calculated using cells from the same cluster, individual, and replicate. To identify marker genes expressed in each cluster, Limma and voom were used to perform differential expression analysis using the pseudobulk estimates. For example, considering the gene expression data of the seven clusters identified at resolution 0.1, the most significantly upregulated genes in each cluster were found to include known marker genes for pluripotent cells (cluster 0), early ectoderm (cluster 1), mesoderm (cluster 2), neural crest (cluster 3), endoderm (cluster 4), neurons (cluster 5), and endothelial cells (cluster 6) (Table 2). Using this approach provides a confident set of broad cell type categories present in these data. At higher resolutions, DE analysis between clusters enabled annotation of some more specific cell types; for example, at clustering resolution 1, cluster 19 is characterized by higher expression of hepatocyte marker genes FGB, TTR, and AFP. More generally, however, confident cell type classification of Seurat clusters at higher resolution based on DE alone proved difficult.

TABLE 2
Classification of Seurat cluster identity (clustering resolution 0.1) based on differential
expression of marker genes.

| Cluster | # cells in | Top 10 marker genes by | Top 10 marker genes by | |
|---|---|---|---|---|
| (Res. 0.1) | cluster | adj. P | logFC | Annotation |
| 0 | 17,693 | TERF1, PHC1, SEPHS1, | DPPA5, DPPA3, GDF3, | Pluripotent |
| | | UGP2, DPPA4, TBC1D23, | NANOG, FGF4, POU5F1, | Cells |
| | | JARID2, USO1, ZNF398, | CBR3, PRDM14, DPPA2, | |
| | | LRRC47 | TRIML2 | |
| 1 | 14,383 | TPBG, FGFBP3, FZD3, | FEZF2, EMX2, LHX2, | Early |
| | | LIX1, SDK2, BTBD17, | SOX3, PAX6, WNT7, | Ectoderm |
| | | DACH1, PLAGL1, DEK, | BARX, SOX1, ZIC1, SIX3 | |
| | | ZNF219 | | |
| 2 | 3086 | TNNI1, COL6A3, COL5A1, | RGS13, LUM, TECRL, | Mesoderm |
| | | RGS4, ACTA2, TMEM88, | DCN, HAND1, PITX1, | |
| | | DOK4, SLC40A1, HAND2, | COL3A1, SLN, IGF2, FIBIN | |
| | | COL3A1 | | |
| 3 | 2673 | NR2F1, CNP, S100B, | MPZ, PRSS56, ROPN1, | Neural Crest |
| | | EDNRA, FGFBP3, ATP1A2, | SOX10, S100B, SCRG1, | |
| | | DNAJC1, ZMTO2, | NPR3, MOXD1, TF AP2B, | |
| | | PHACTR3, METRN | PHACTR3 | |
| 4 | 2368 | S100A16, LGALS3, GATA3, | APOA2, CST1, APOA1, | Endoderm |
| | | CST3, KRT19, FN1, EPSTI1, | APOC3, FGB, RBP4, | |
| | | DYNLT3, HDHD3, PKP2 | S100A14, TTR, FGA, APOB | |
| 5 | 1990 | TAGLN3, RTN1, NHLH1, | NEUROD1, NHLH1, | Neurons |
| | | STMN2, ELAVL2, FNDC5, | STMN2, NEUROD4, TBR1, | |
| | | PCBP4, ELAVL4, DCX, | STMN4, NEUROG1, SST, | |
| | | MLLT11 | ELAVL3, SLC17A6 | |
| 6 | 295 | EGFL7, GNG11, RAMP2, | PLVAP, CD34, CD93, | Endothelial |
| | | IGFBP4, PPM1F, RASGRP3, | CDH5, DIPK2B, PECAM1, | Cells |
| | | RCSD1, MAP4K2, PLVAP, | EMCN, CRHBP, ESAM, | |
| | | DOCK6 | ECSCR | |

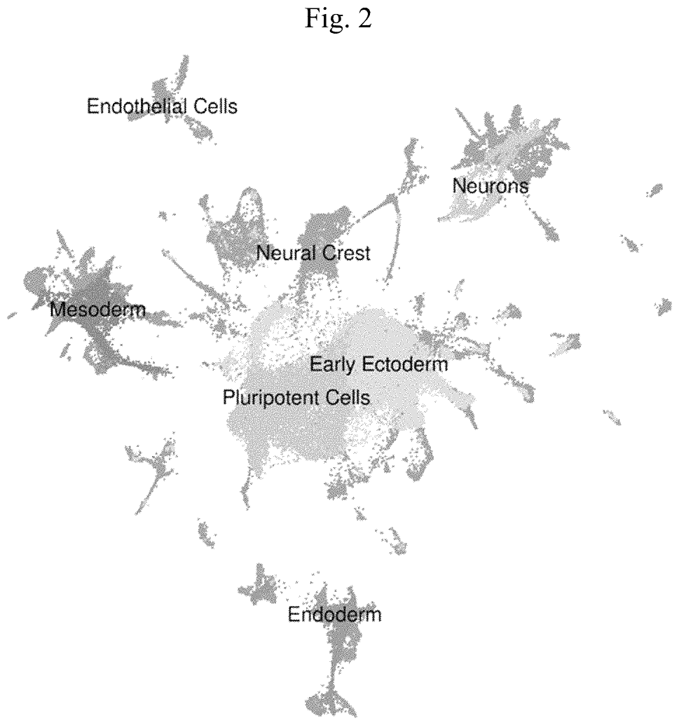

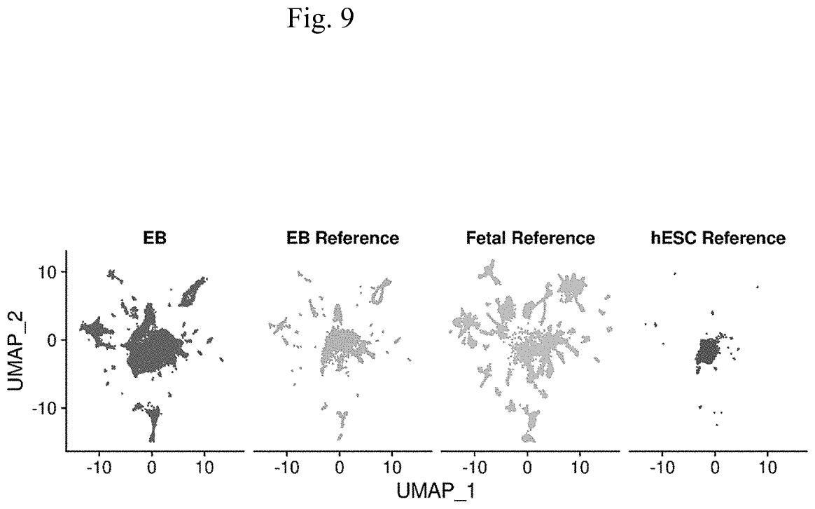

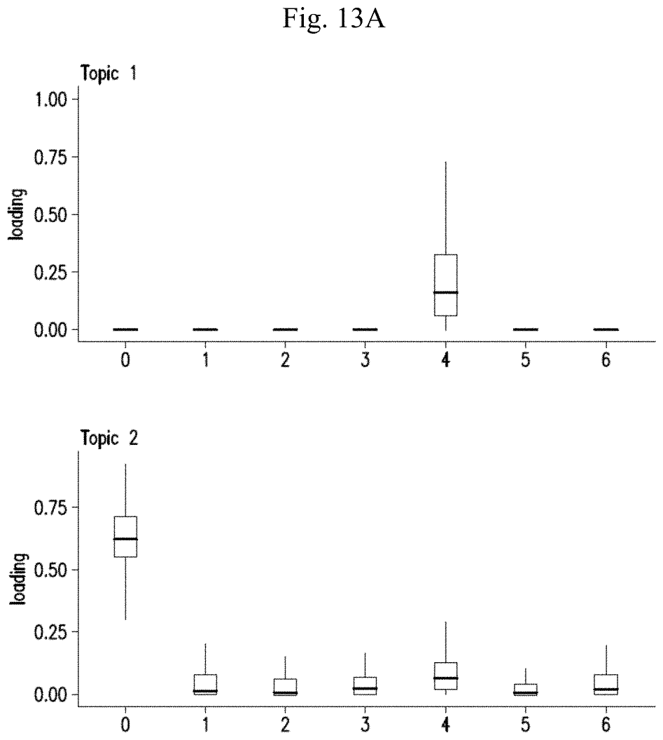

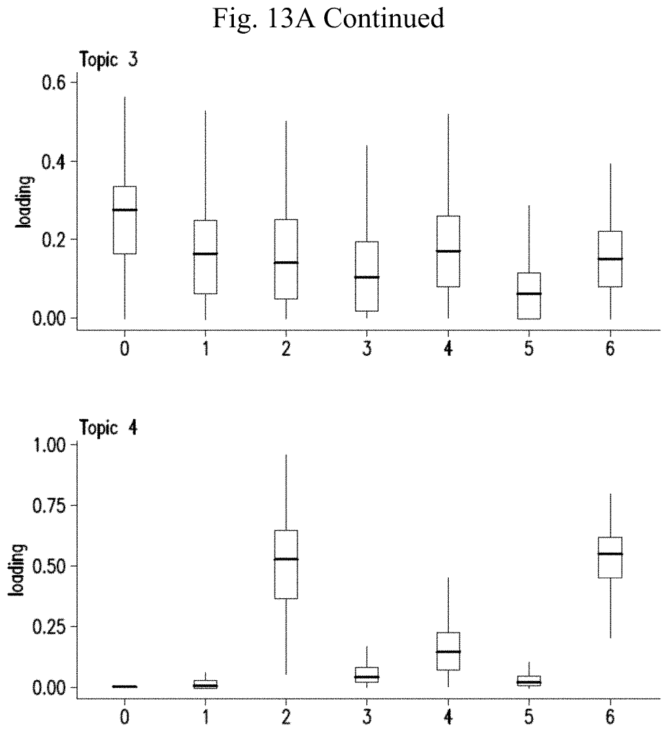

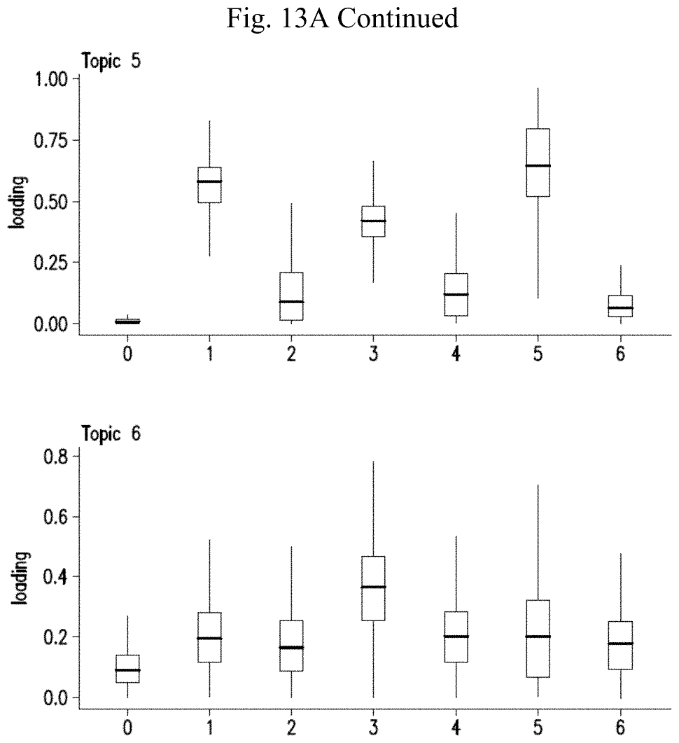

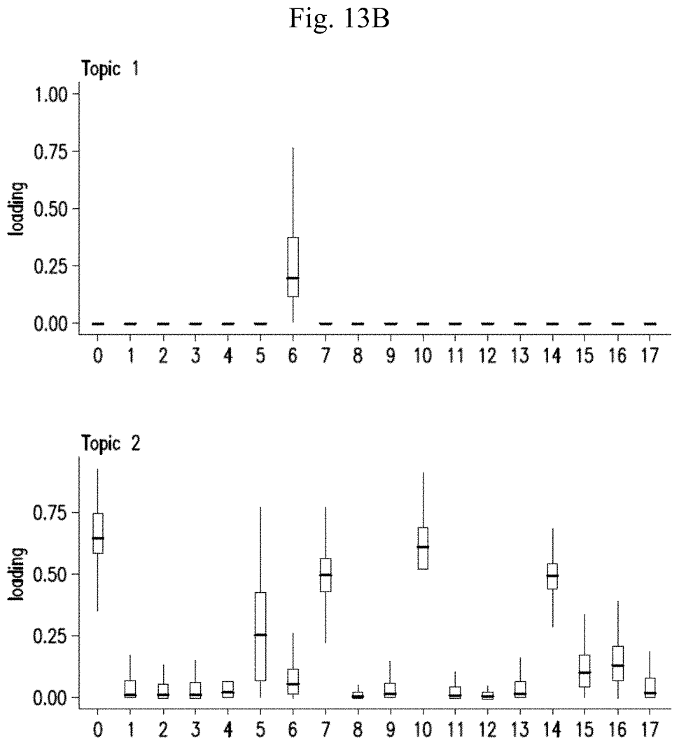

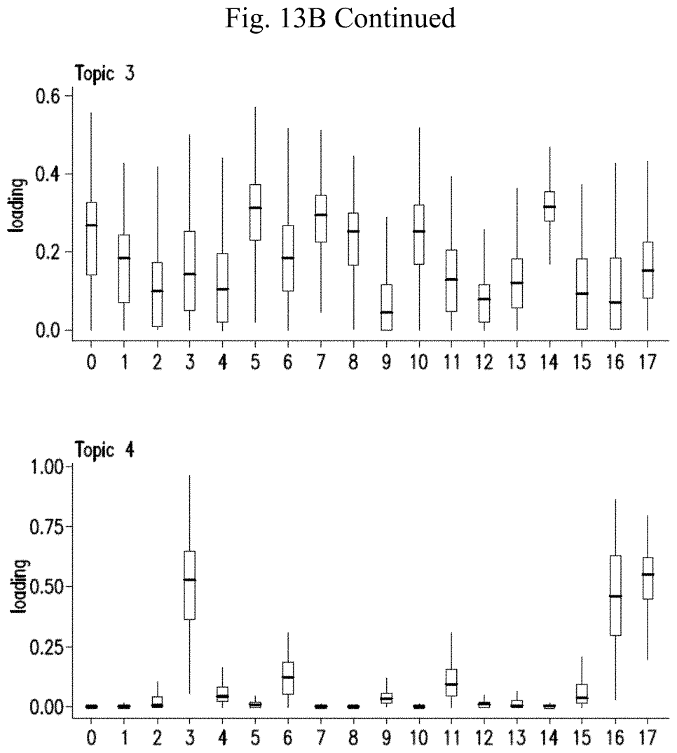

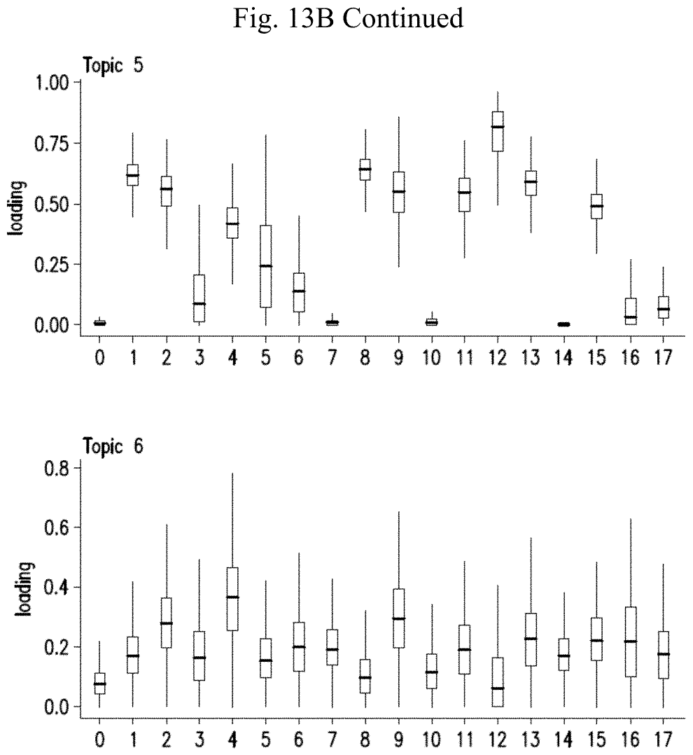

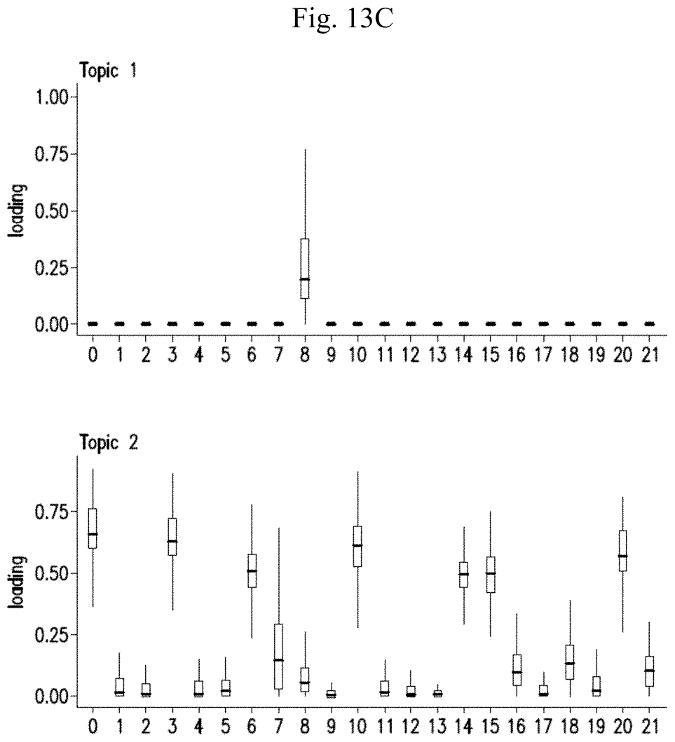

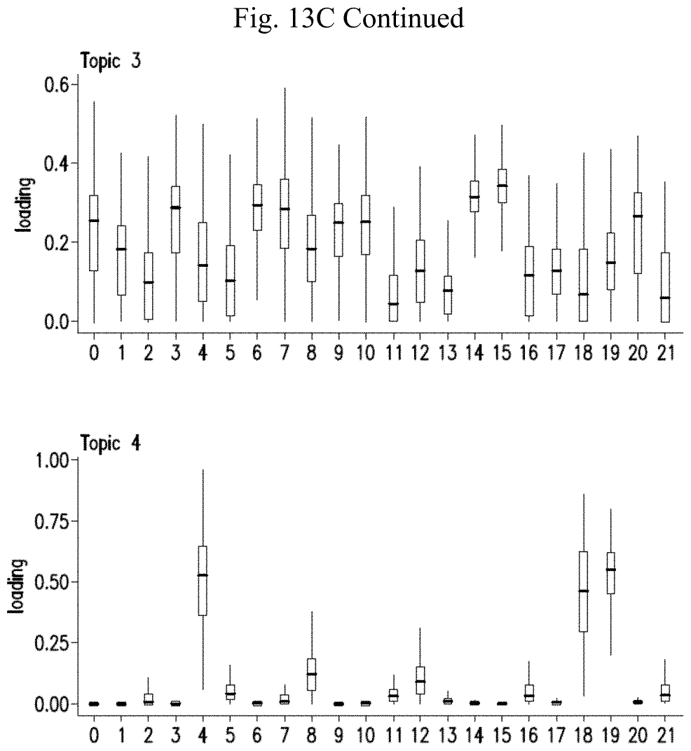

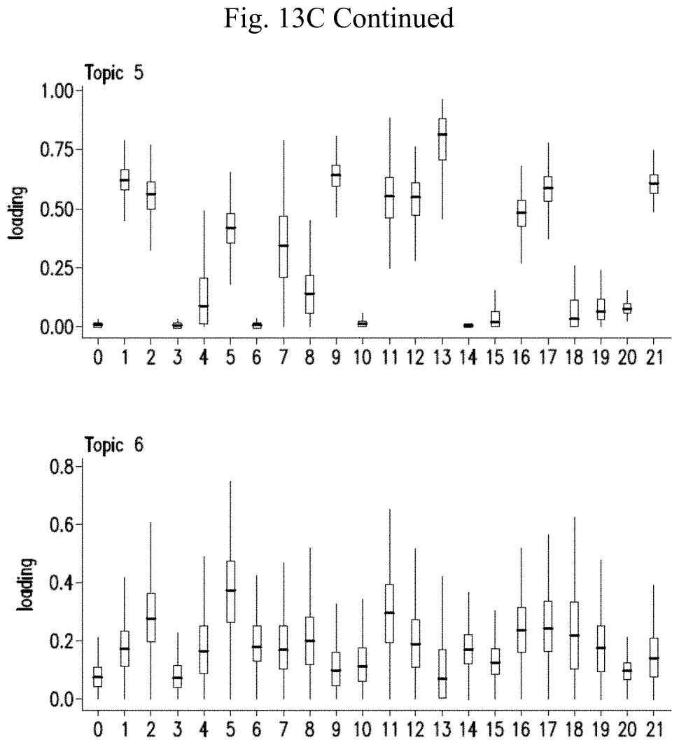

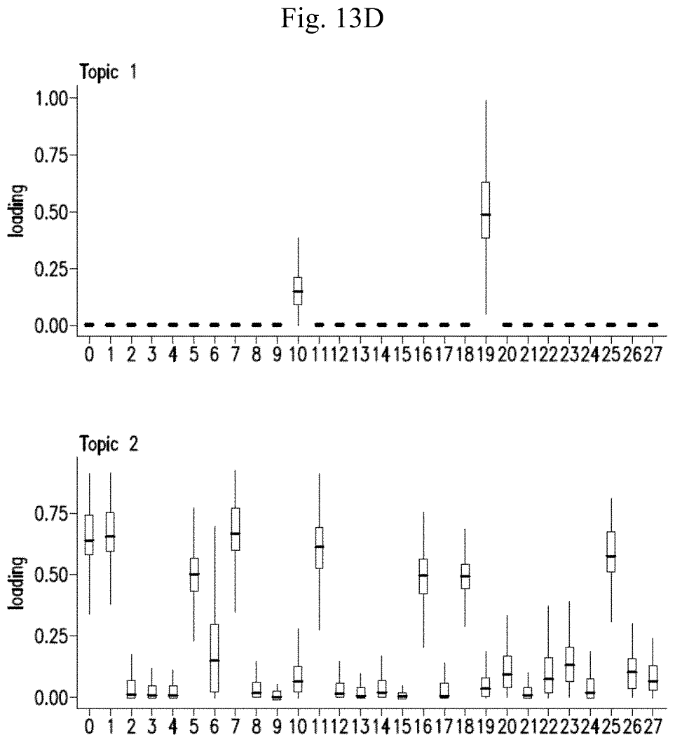

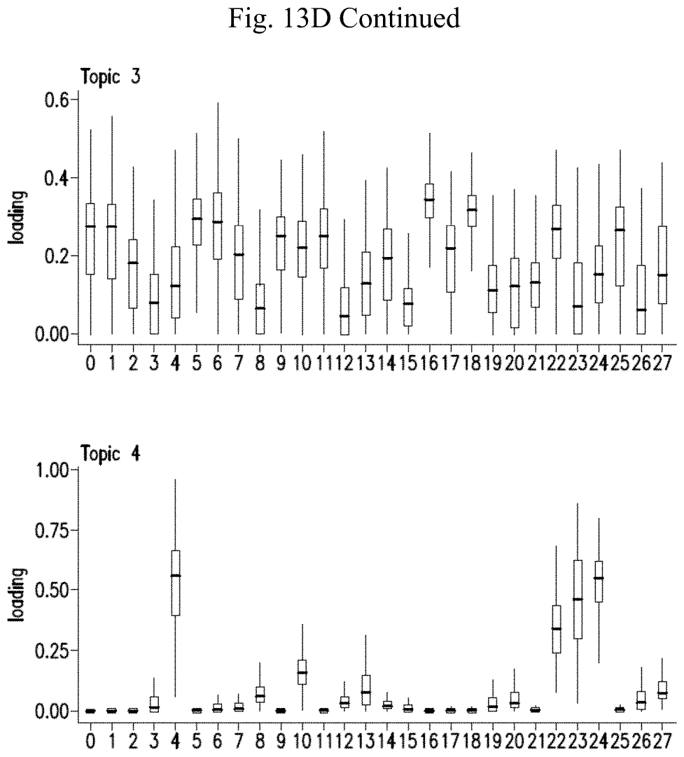

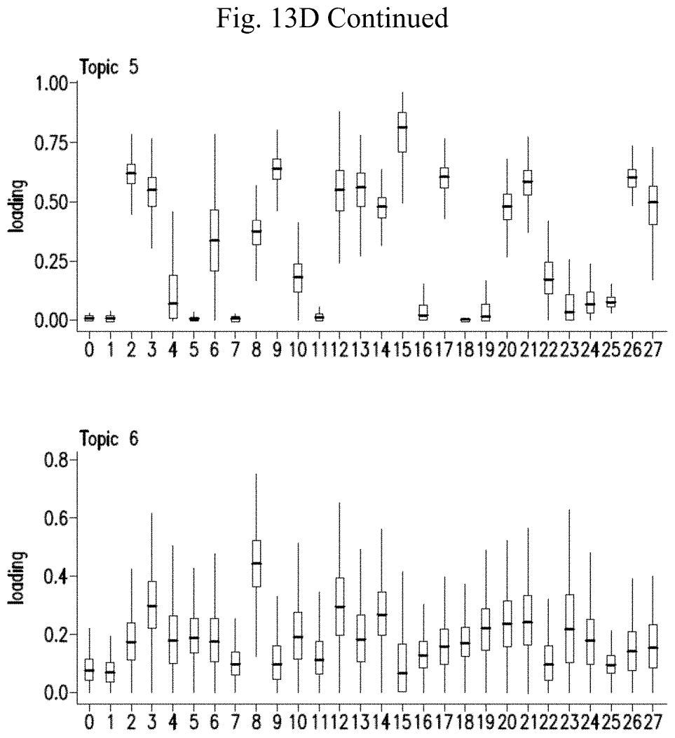

To pursue the second approach, cells were annotated by comparing the gene expression data to available reference sets of scRNA-seq data from primary fetal tissues, human embryonic stem cells (hESCs), and hESC-derived MTOs (Cao et al., 2020; Han et al., 2020). To do this, the data set was first integrated with the reference data sets and cells were visualized with UMAP (FIG. 9). Reference hESCs cluster closely with pluripotent MTO cells. The hESC-derived MTOs and the iPSC-derived MTOs tend to occupy the same areas in UMAP space, implying high overall similarity in cell type composition despite differing protocols for MTO differentiation (and despite the fact that the experiments were performed in different labs). MTO cells also show overlap with many primary fetal cell types (FIGS. 2 and 13B and Table 3). For example, MTO cells annotated as neural crest based on gene expression analysis, overlap with primary fetal cell types derived from neural crest, such as Schwann cells and enteric nervous system (ENS) glia (FIG. 2). MTO cells annotated as neurons based on gene expression analysis overlap with fetal neuronal subtypes, including inhibitory neurons, excitatory neurons, granule neurons, ENS neurons, and others. MTO cells also show overlap with populations of cells that are rare in the fetal data set, including AFP_ALB positive cells (hepatic cells), thymic epithelial cells, and lens fibre cells.

The annotation of MTO cells (which up to this point were based on the expression of known marker genes) was then expanded by using the known annotations of the reference primary fetal cell type data set. Specifically, cell annotations were transferred to MTO cells based on the nearest reference cells in harmony-corrected PCA space. Using this approach, MTO cells were found representing 66 of the 77 primary cell types present in the reference fetal data set (Table 3). Overall, annotation based on the reference set revealed the presence of dozens of diverse cell types in MTOs.

TABLE 3

| | Frequency in MTO |
|---|---|
| Cell Type Annotation | cells |
| Acinar cells | 52 |
| Adrenocortical cells | 7 |
| AFP_ALB positive cells | 36 |
| Amacrine cells | 79 |
| Antigen presenting cells | 1 |
| Astrocytes | 39 |
| Bipolar cells | 40 |
| Cardiomyocytes | 521 |
| CCL19_CCL21 positive cells | 2 |
| Chromaffin cells | 24 |
| Ciliated epithelial cells | 314 |
| CLC_IL5RA positive cells | 3 |
| Corneal and conjunctival epithelial cells | 21 |
| Ductal cells | 329 |
| ELF3_AGBL2 positive cells | 23 |
| Endocardial cells | 5 |
| ENS glia | 30 |
| ENS neurons | 72 |
| Epicardial fat cells | 112 |
| Erythroblasts | 34 |
| Excitatory neurons | 1 |
| Ganglion cells | 61 |
| Goblet cells | 271 |
| Granule neurons | 158 |
| Hematopoietic stem cells | 9 |
| Hepatoblasts | 212 |
| Horizontal cells | 53 |
| IGFBP1_DKK1 positive cells | 341 |
| Inhibitory interneurons | 9 |
| Inhibitory neurons | 65 |
| Intestinal epithelial cells | 501 |
| Islet endocrine cells | 552 |
| Lens fibre cells | 51 |
| Limbic system neurons | 15 |
| Lymphatic endothelial cells | 2 |
| Megakaryocytes | 37 |
| Mesangial cells | 222 |
| Mesothelial cells | 160 |
| Metanephric cells | 142 |
| Microglia | 44 |
| MUC13_DMBT1 positive cells | 181 |
| Myeloid cells | 1 |
| Neuroendocrine cells | 4 |
| Oligodendrocytes | 53 |
| Parietal and chief cells | 14 |
| PDE11A_FAM19A2 positive cells | 35 |
| Photoreceptor cells | 9 |
| Purkinje neurons | 35 |
| Retinal pigment cells | 391 |
| Retinal progenitors and | 21 |
| Muller glia | |
| scHCL.hESC | 34086 |
| Schwann cells | 38 |
| Skeletal muscle cells | 5 |
| SKOR2_NPSR1 positive cells | 39 |
| SLC24A4_PEX5L positive cells | 297 |
| Smooth muscle cells | 60 |
| Squamous epithelial cells | 160 |
| Stellate cells | 247 |
| Stromal cells | 217 |
| Syncytiotrophoblasts and villous | 17 |
| cytotrophoblasts | |
| Thymic epithelial cells | 116 |
| Thymocytes | 1 |
| Trophoblast giant cells | 5 |
| uncertain | 1566 |
| Unipolar brush cells | 37 |
| Ureteric bud cells | 26 |
| Vascular endothelial cells | 58 |
| Visceral neurons | 119 |

### Differentiation Efficiency Across Individuals

To assess the differentiation efficiency of each individual in each replicate, the proportion of cells assigned to each cluster as resolution 0.1 was calculated (FIG. 1E). While MTOs from two of the individuals in this study differentiated efficiently across all replicates, 89% of cells from individual 18858 were assigned to cluster 0, the cluster annotated as pluripotent cells based on differential expression of marker genes (Table 2 and Table 4). The MTOs from this line do differentiate, producing high quality cells assigned to clusters representing each germ layer (FIG. 1E), but these MTO have overall markedly lower differentiation efficiency than MTOs from individuals 18511 and 19160. To determine whether individual 18858 is an outlier, and more generally estimate how often MTO differentiation is less efficient, an additional five human iPSC lines from individuals 18856, 18912, 19140, 19159, and 19210 were differentiated. MTOs from additional lines differentiated efficiently, with cell type composition similar to 18511 and 19160 (FIG. 1F). These results suggest that poor differentiation efficiency is expected to be rare among the YRI iPSC panel. The robustness of cell type composition was further explored by integrating the additional lines with the fetal and hESC reference data sets using the same methodology as was used for the original lines. Annotations assigned to cells from these additional lines represented 66 of the 77 fetal cell types; this set of annotations included several cell types that were missing in the original three lines, but also excludes several that were seen in the original three lines (Table 5). Again, most MTO cells from the additional lines are annotated as hESC (82% of cells), although many no longer express pluripotency markers and do express markers of various germ layers as previously observed. Together, these results support the conclusions that MTOs contain many diverse cell types, many of which likely capture earlier stages of development than are captured in fetal data.

TABLE 4

| | Cluster | | | | | | |
|---|---|---|---|---|---|---|---|
| Individuals | 0 | 1 | 2 | 3 | 4 | 5 | 6 |
| 18511_Rep1 | 99 | 2918 | 423 | 139 | 277 | 278 | 37 |
| 18511_Rep2 | 136 | 1958 | 35148 | 35 | 212 | 193 | 17 |
| 18511_Rep3 | 1508 | 2514 | 372 | 128 | 272 | 250 | 35 |
| 18858_Rep1 | 5995 | 526 | 29 | 28 | 116 | 35 | 3 |
| 18858_Rep2 | 4005 | 703 | 4 | 3 | 24 | 9 | 0 |
| 18858_Rep3 | 4882 | 235 | 5 | 9 | 47 | 12 | 0 |
| 19160_Rep1 | 81 | 2870 | 997 | 1129 | 196 | 634 | 63 |
| 19160_Rep2 | 179 | 1025 | 361 | 383 | 221 | 207 | 42 |
| 19160_Rep3 | 808 | 1634 | 747 | 819 | 1003 | 372 | 98 |

TABLE 5

| | Frequency in |
|---|---|
| Cell Type Annotation | MTOs |
| Acinar cells | 8 |
| Adrenocortical cells | 10 |
| AFP_ALB positive cells | 9 |
| Amacrine cells | 13 |
| Astrocytes | 71 |
| Bipolar cells | 35 |
| Bronchiolar and alveolar epithelial | 9 |
| cells | |
| Cardiomyocytes | 89 |
| Chromaffin cells | 35 |
| Ciliated epithelial cells | 70 |
| CLC_IL5RA positive cells | 1 |
| Ductal cells | 5 |
| ELF3_AGBL2 positive cells | 3 |
| Endocardial cells | 11 |
| ENS glia | 177 |
| ENS neurons | 22 |
| Epicardial fat cells | 3 |
| Erythroblasts | 19 |
| Excitatory neurons | 33 |
| Extravillous trophoblasts | 8 |
| Ganglion cells | 106 |
| Granule neurons | 775 |
| Hematopoietic stem cells | 1 |
| Hepatoblasts | 398 |
| Horizontal cells | 53 |
| IGFBP1_DKK1 positive cells | 17 |
| Inhibitory interneurons | 11 |
| Inhibitory neurons | 86 |
| Intestinal epithelial cells | 6 |
| Islet endocrine cells | 44 |
| Lens fibre cells | 5 |
| Limbic system neurons | 70 |
| Lymphatic endothelial cells | 9 |
| Lymphoid cells | 1 |
| Megakaryocytes | 14 |
| Mesangial cells | 101 |
| Mesothelial cells | 1 |
| Metanephric cells | 58 |
| Microglia | 46 |
| MUC13_DMBT1 positive cells | 2 |
| Myeloid cells | 2 |
| Neuroendocrine cells | 10 |
| Oligodendrocytes | 44 |
| PAEP_MECOM positive cells | 7 |
| Parietal and chief cells | 2 |
| PDE11A_FAM19A2 positive cells | 49 |
| Photoreceptor cells | 21 |
| Purkinje neurons | 25 |
| Retinal pigment cells | 90 |
| Retinal progenitors and Muller glia | 77 |
| scHCL.hESC | 21724 |
| Schwann cells | 70 |
| Skeletal muscle cells | 12 |
| SKOR2_NPSR1 positive cells | 29 |
| SLC24A4_PEX5L positive cells | 37 |
| Smooth muscle cells | 3 |
| Squamous epithelial cells | 32 |
| Stellate cells | 32 |
| Stromal cells | 448 |
| Sympathoblasts | 11 |
| Thymic epithelial cells | 5 |
| Thymocytes | 4 |
| Trophoblast giant cells | 8 |
| uncertain | 873 |
| Unipolar brush cells | 38 |
| Ureteric bud cells | 13 |
| Vascular endothelial cells | 59 |
| Visceral neurons | 46 |

### Topic Modeling of the Single-Cell Gene Expression Data

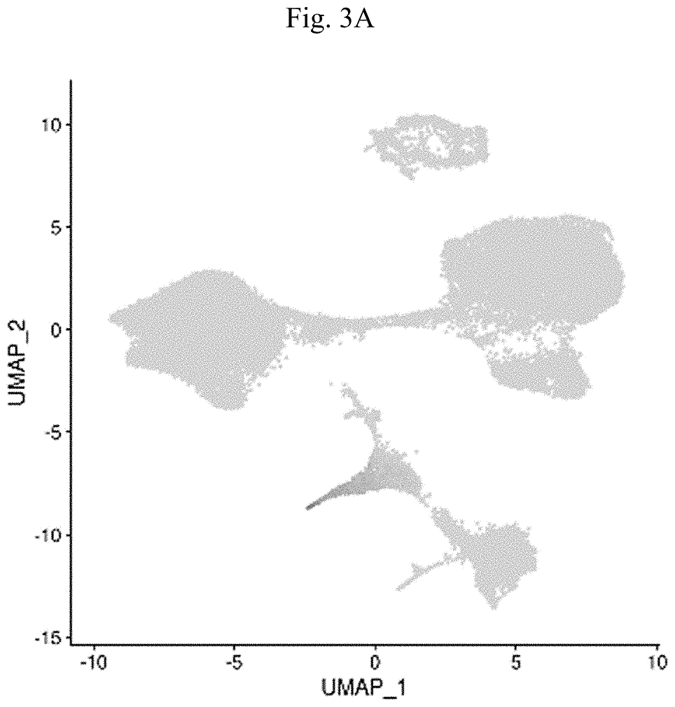

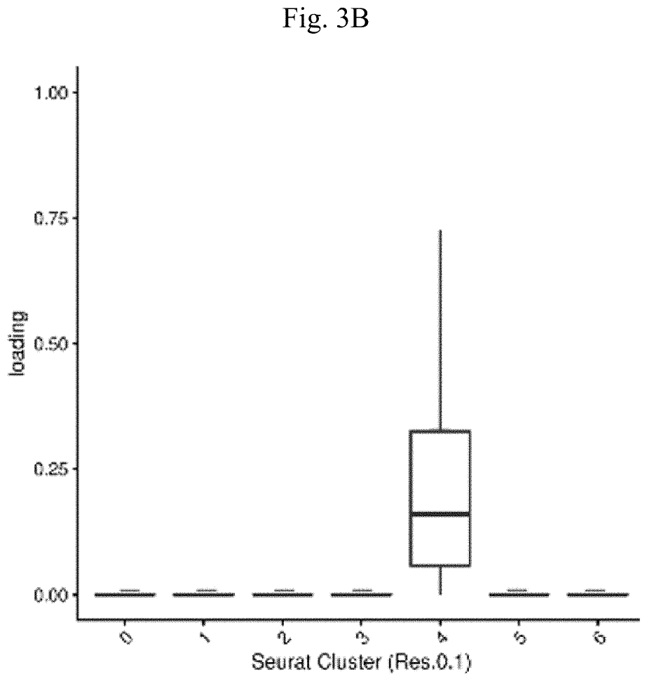

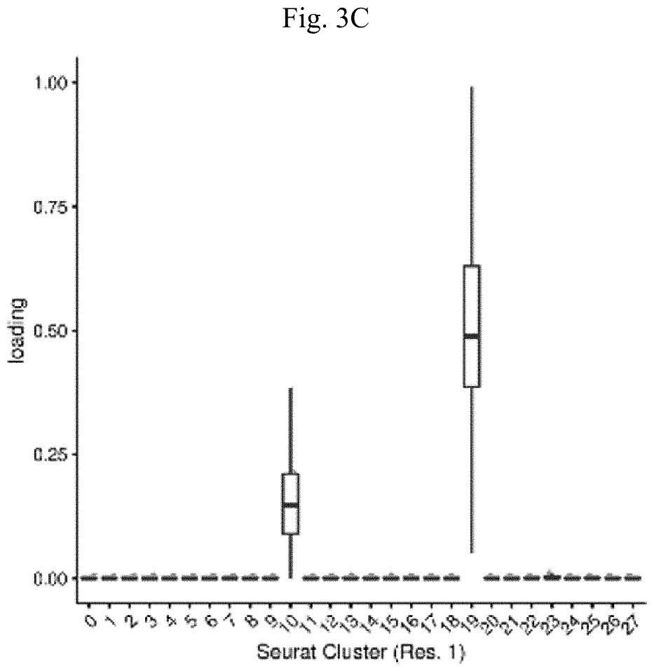

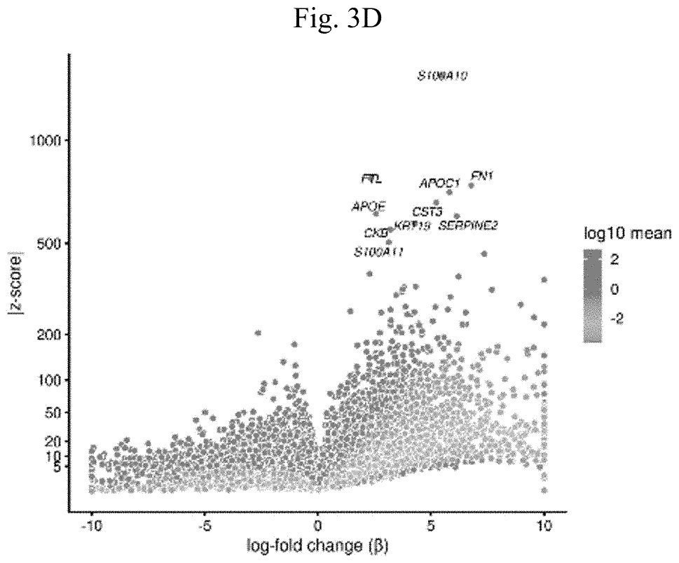

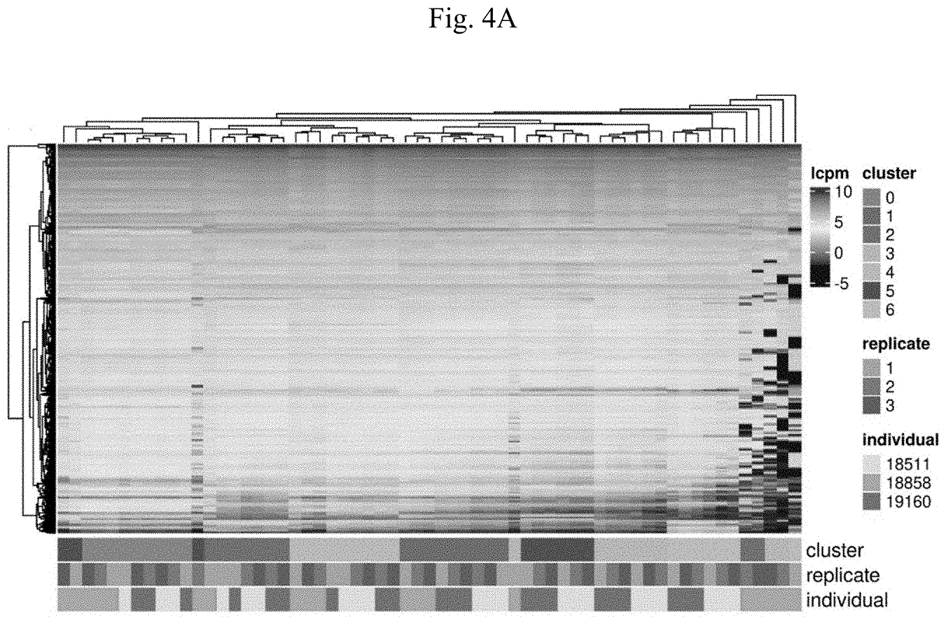

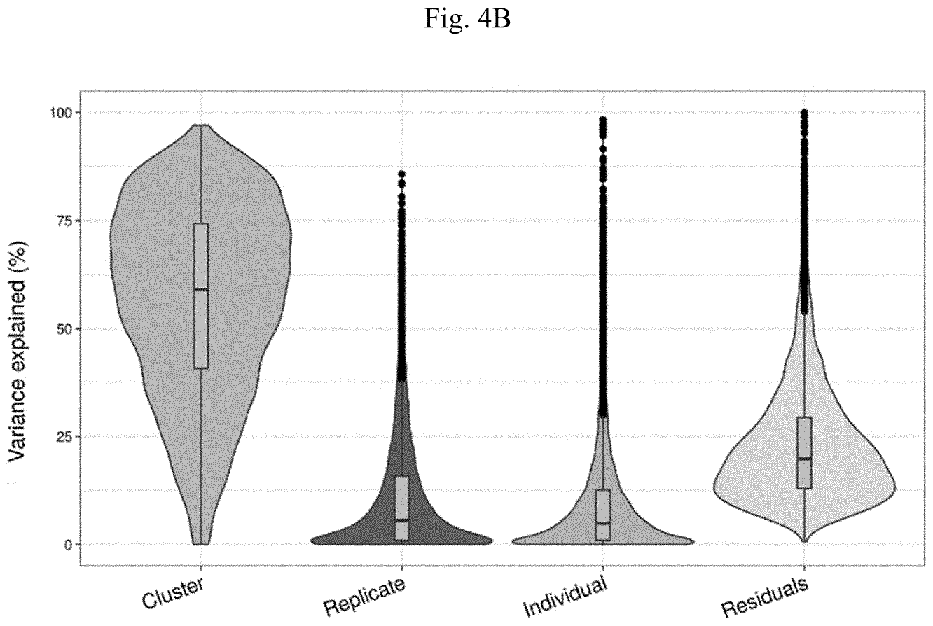

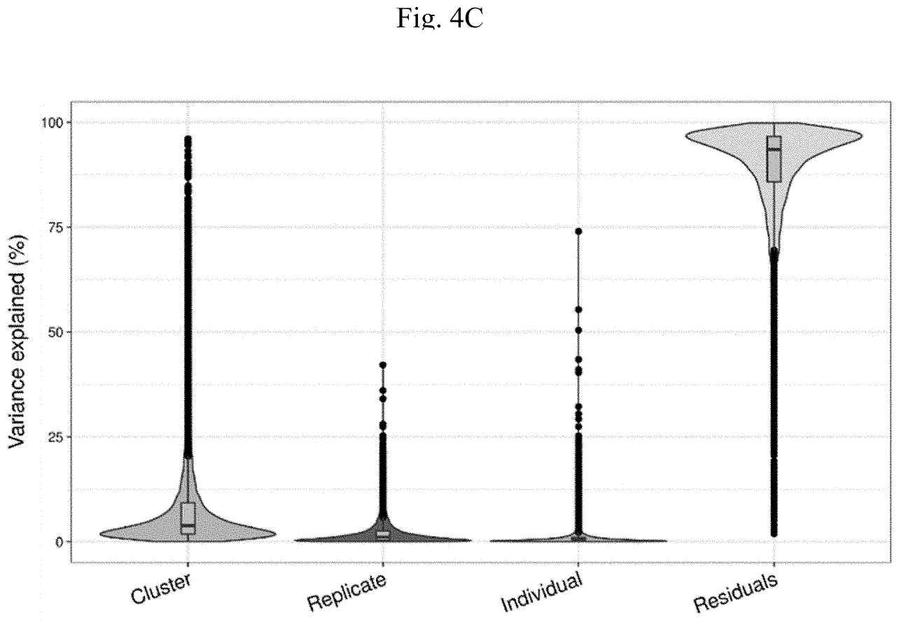

Both of the approaches described above (clustering, and comparison to a reference data set) assume that ‘cell types’ are discrete categories. Accordingly, each cell has a single true identity, and cell type categories are assumed to be homogeneous and static. This definition of a cell type is intuitive and makes it practical to consider results from single-cell analysis in the context of the wealth of knowledge previously gained from bulk assays. However, partitioning cells into discrete groups is unlikely to capture the full degree of heterogeneity in gene expression of single cells. A particular cluster or cell ‘type’ may collapse multiple cell states, obscuring functionally distinct subgroups such as cells in different stages of the cell cycle. This problem becomes more apparent in data sets that include differentiating cells, which are expected to show varying degrees of similarity to a terminal cell type. In an alternate paradigm, cell type can be viewed as continuous, with the expression profile of each cell representing grades of membership to multiple categories (Dey et al., 2017). One method used to capture cell identity in this paradigm is topic modeling, which learns major patterns in gene expression within the data set, or topics, and models each cell as a combination of these topics. Topic modeling was applied using fastTopics at a range of topic resolutions, identifying 6, 10, 15, 25, and 30 topics in this data. Some topics correspond closely to Seurat clusters, loaded on cells of a given cluster but not on others. For example, in the k=6 topic analysis, topic 1 is loaded exclusively on cells assigned to Seurat cluster 4 (cluster resolution 0.1) which was previously annotated as endoderm (FIGS. 3A and 3B and Table 2). Compared to other topics, topic 1 shows an increase in expression of FN1 and AFP, which are known markers of hepatocytes (FIG. 3C and Table 2). Seurat clustering at higher resolution (resolution 1) results in further categorical division of this large endoderm group of cells into definitive endoderm and hepatocytes. Topic modeling revealed that these cells actually exhibit variable grades of membership in topic 1 (in k=6 topic model); this gradient captures a temporal continuum of differentiation.

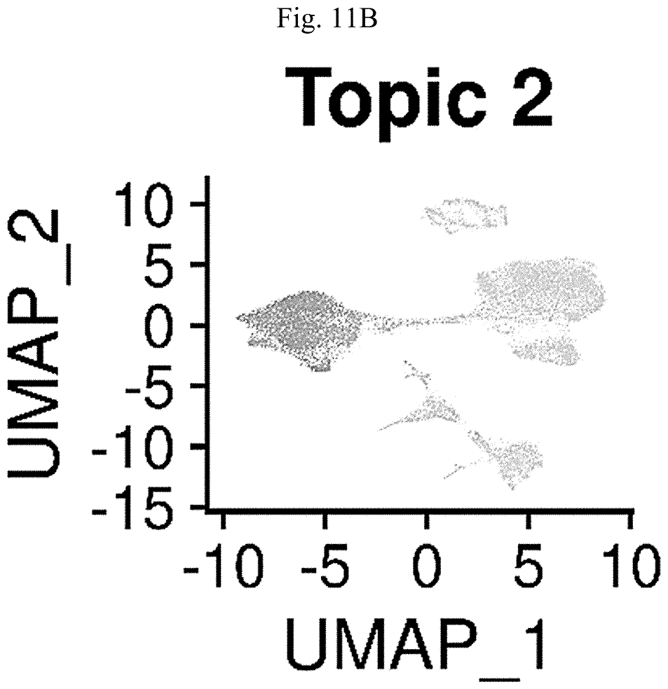

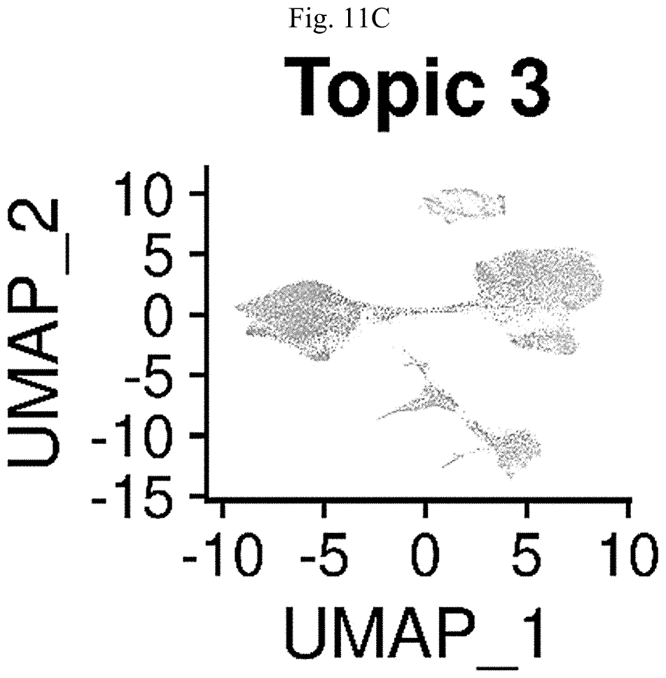

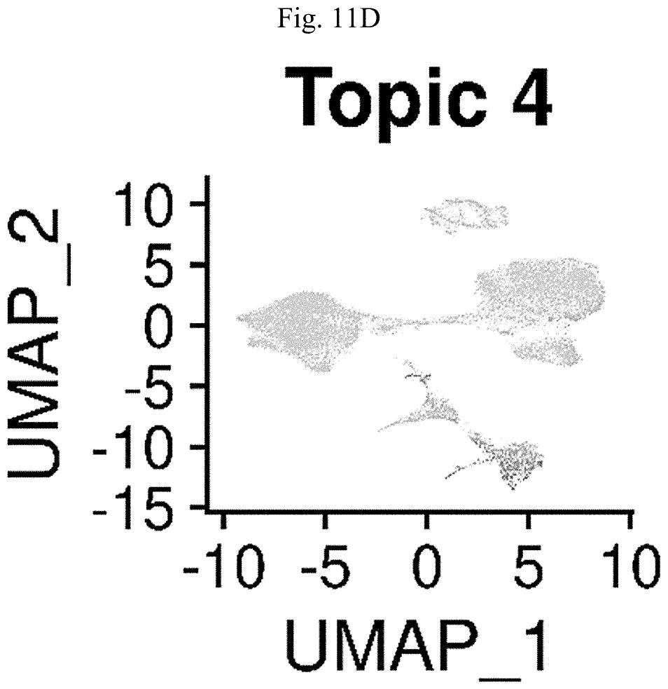

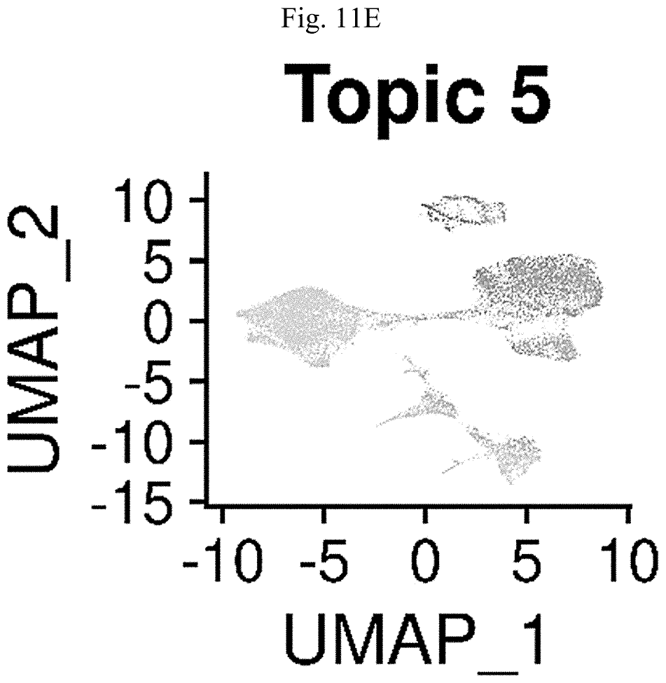

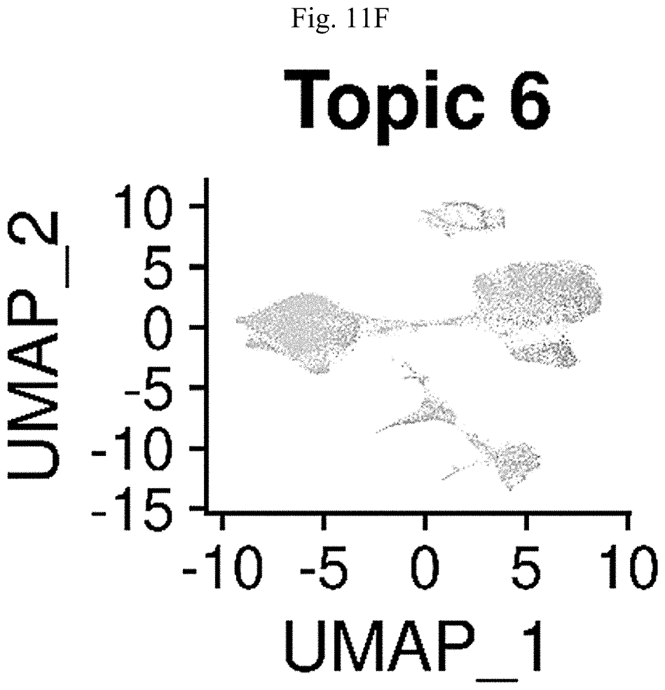

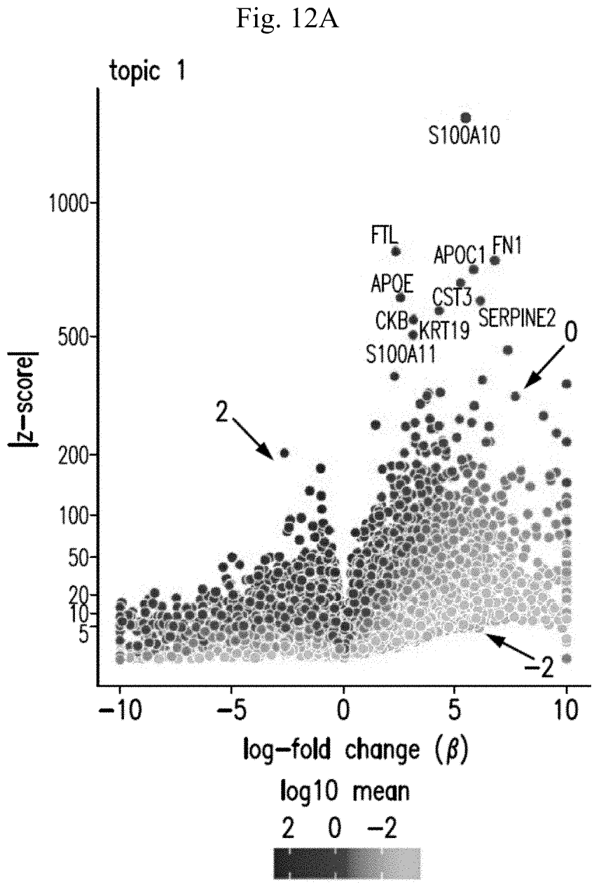

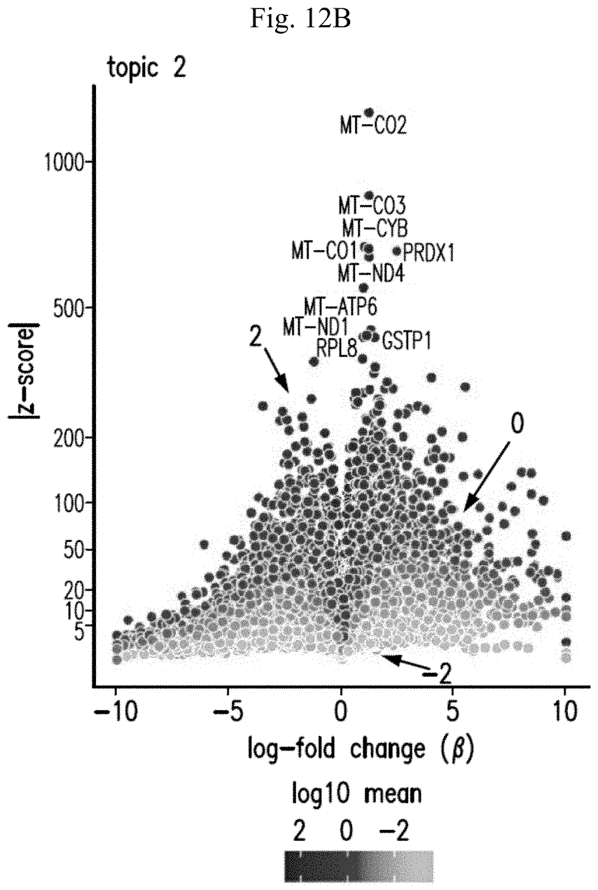

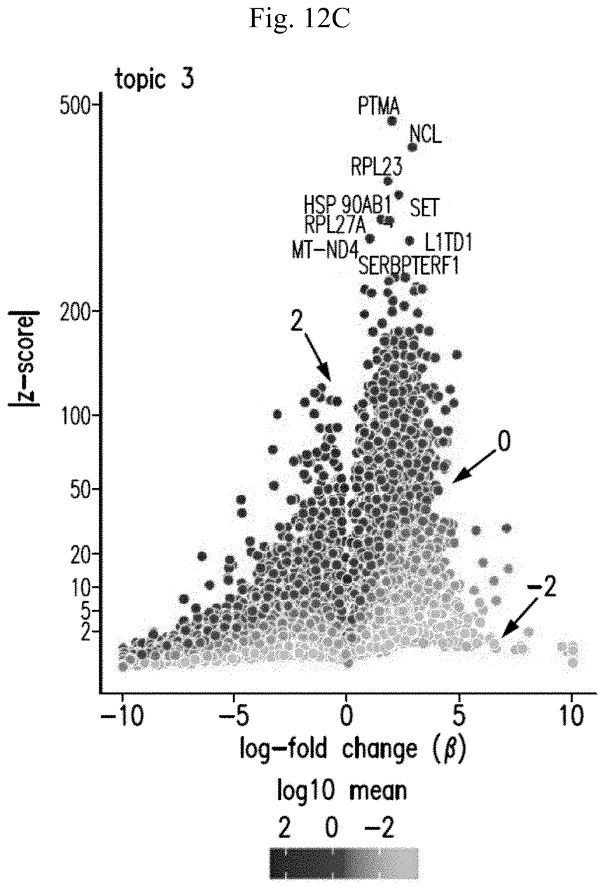

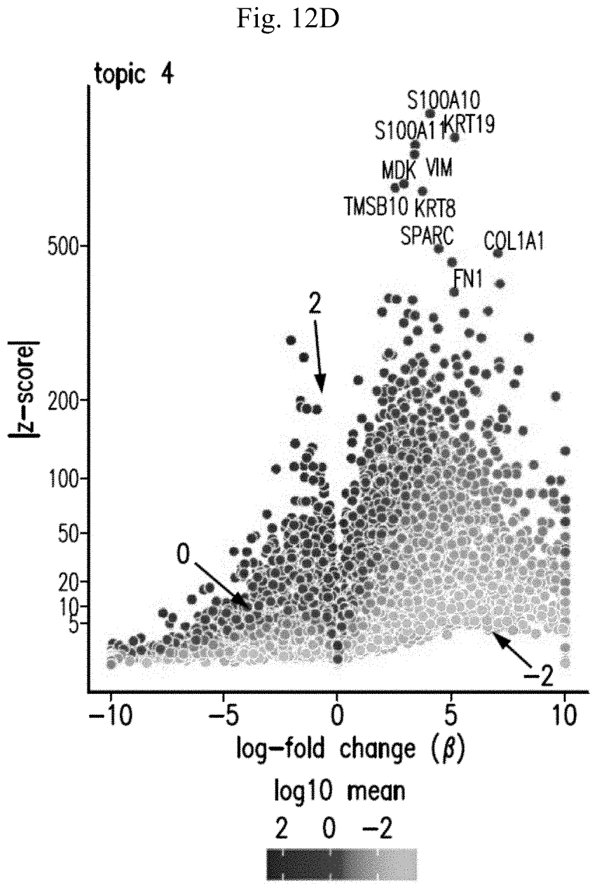

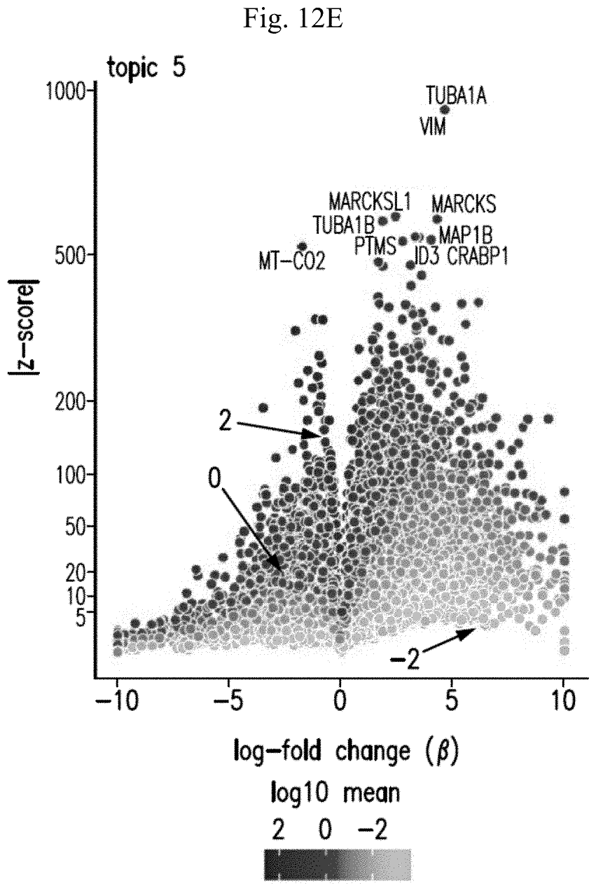

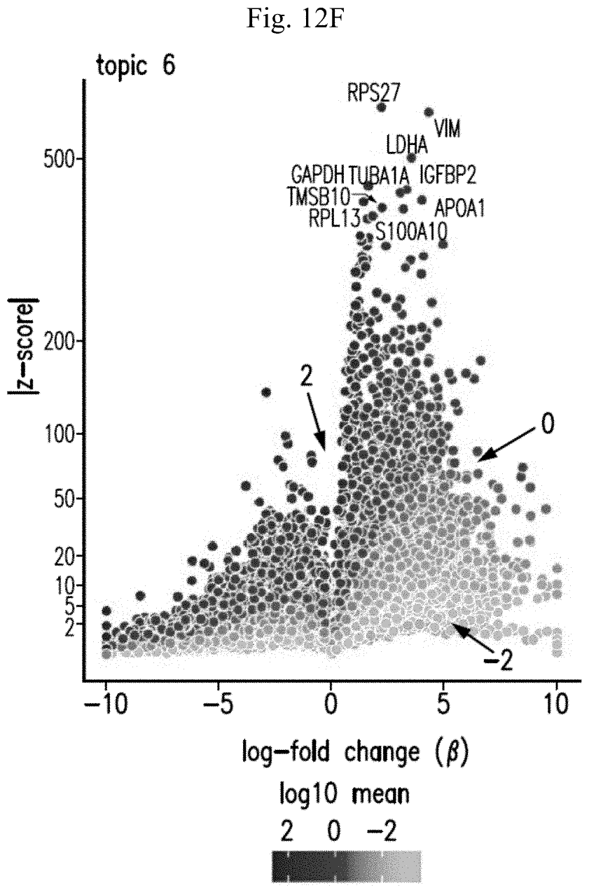

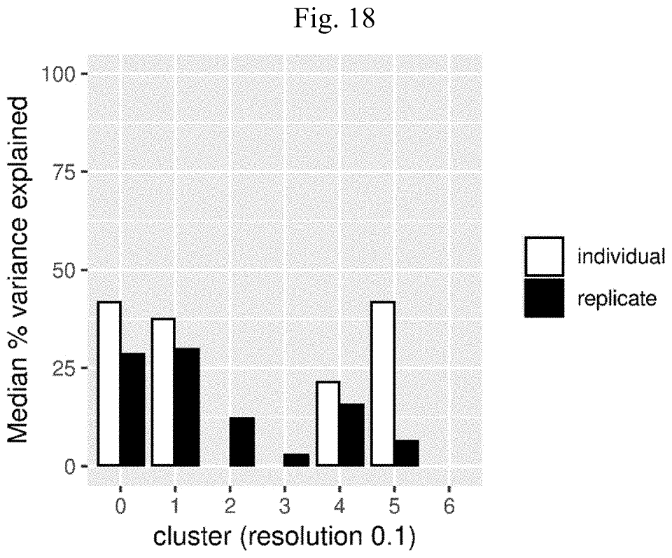

Certain topics are shared across cells assigned to different Seurat clusters (FIGS. 18 A, 18B, 18C, and 18D). For example, topic 6 from the k=6 topic analysis is loaded across all Seurat clusters; compared to all other topics, topic 6 shows increased expression of many ribosomal genes, housekeeping genes (GAPDH), and genes coding for proteins involved in cellular metabolism (LDHA) (FIGS. 11A, 11B, 11C, 11D, 11E, 11F, 12A, 12B, 12C, 12D, 12E, 12F, 13A, 13B, 13C, and 13D). This indicates that topic six captures patterns of gene expression associated with cellular processes and functions that are not specific to a particular cell type. This again highlights an advantage of topic modeling, enabling the exploration of variation in the representation of gene expression profiles associated with processes shared across many cell types, simultaneous with identifying cell-type-specific patterns.

### Biological and Technical Sources of Variation

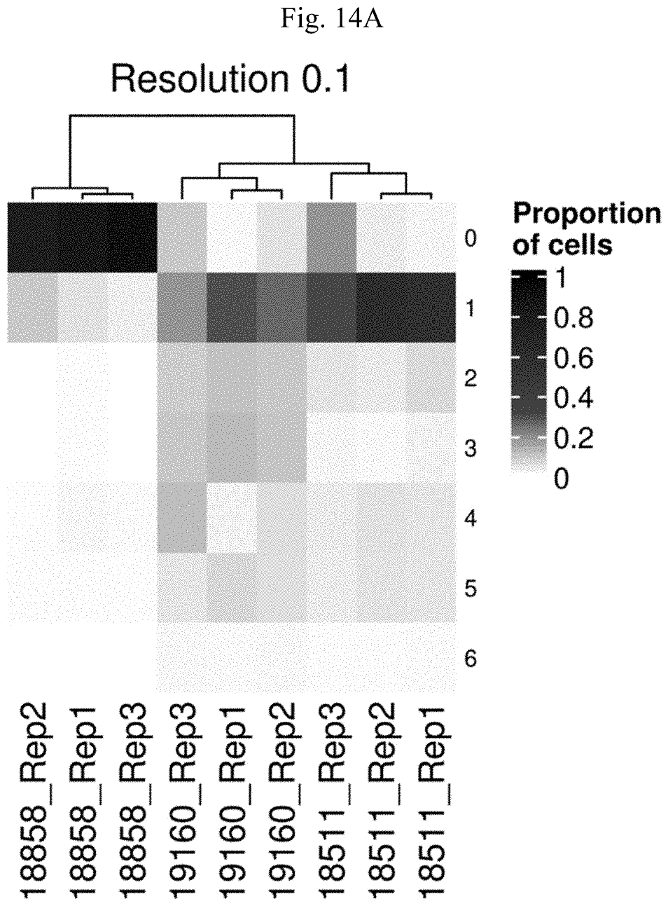

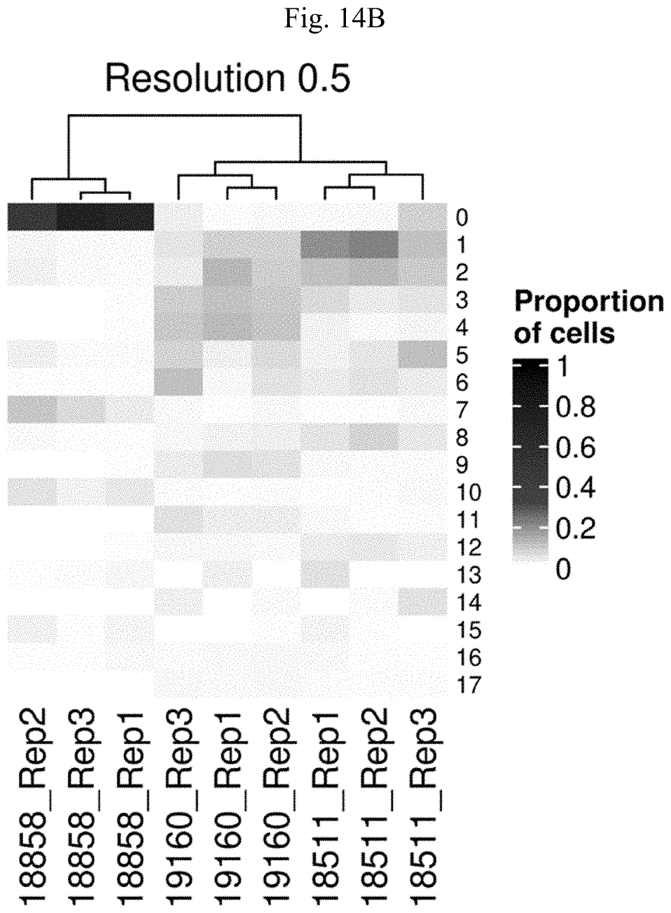

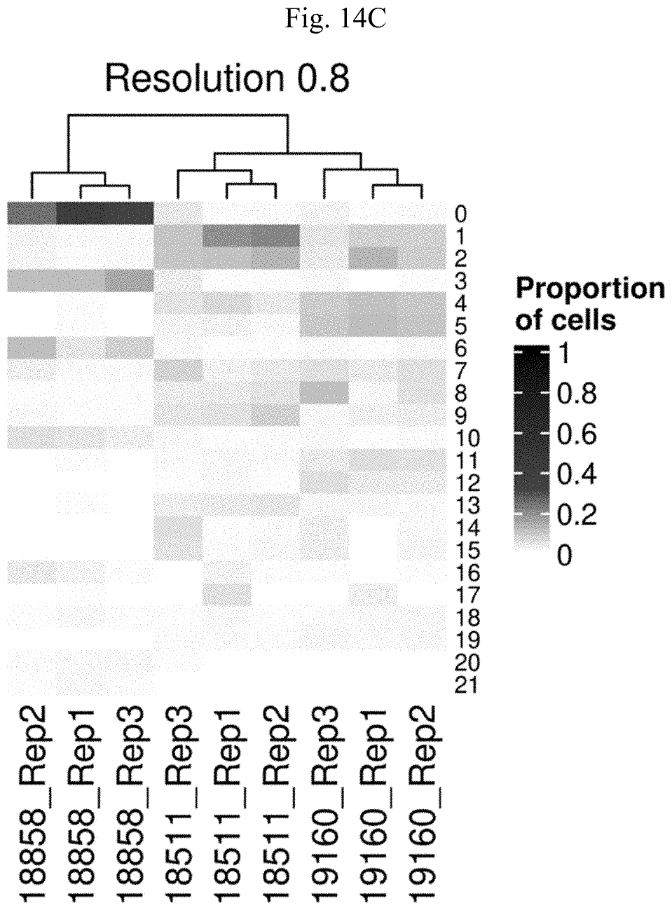

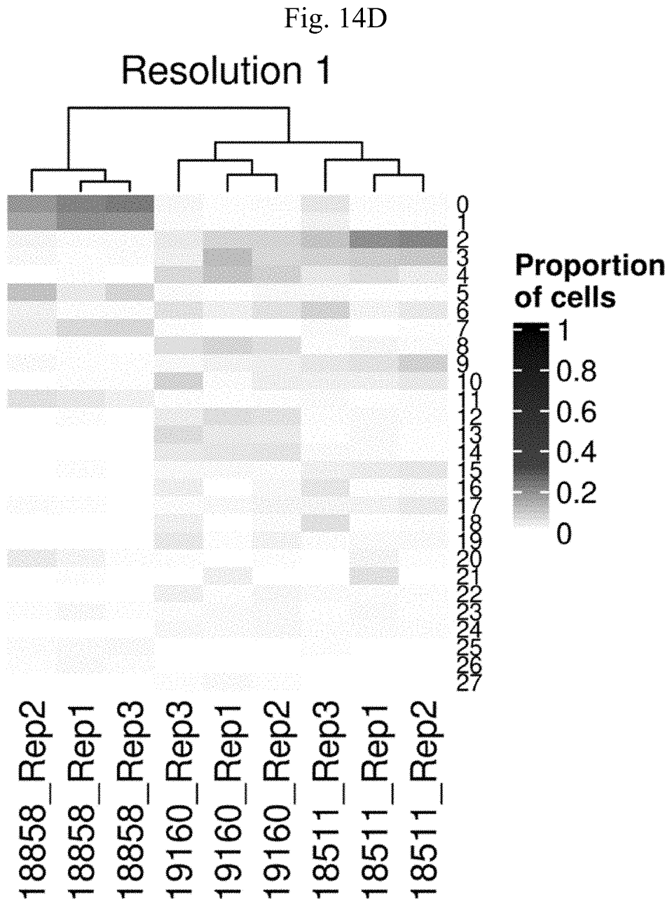

Once MTO cells were functionally annotated using the three approaches discussed above, the consistency in cell type composition across individuals and between replicates was further investigated. Here, ‘replicate’ corresponds to a batch of MTO differentiations in which each cell line was differentiated, dissociated, and collected in tandem; ‘replicate’ therefore captures technical variation related to differentiation batch, dissociation batch, and single-cell collection batch. First, the proportion of cells that were assigned to each Seurat cluster at resolution 0.1 for each replicate was calculated. Then hierarchical clustering of the samples was performed based on the proportion of cells in each Seurat cluster (FIGS. 14A, 14B, 14C, and 14D). Using this approach, replicate-individual samples cluster first by individual, indicating that cell type composition is distinct between individuals and is consistent between replicates of each individual. This analysis was repeated at a range of cluster resolutions and determined that this finding is robust with respect to the number of clusters (FIGS. 14A, 14B, 14C, and 14D).

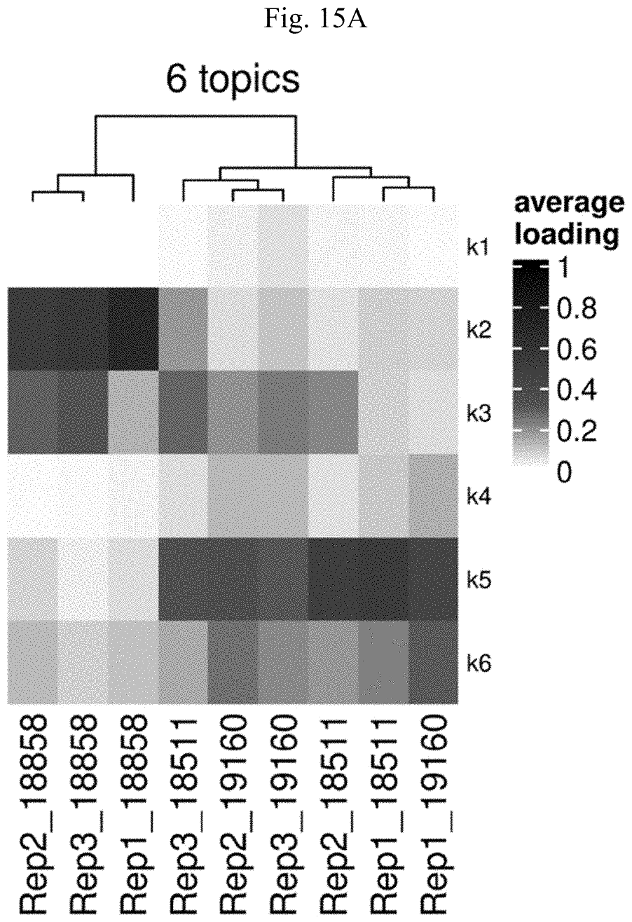

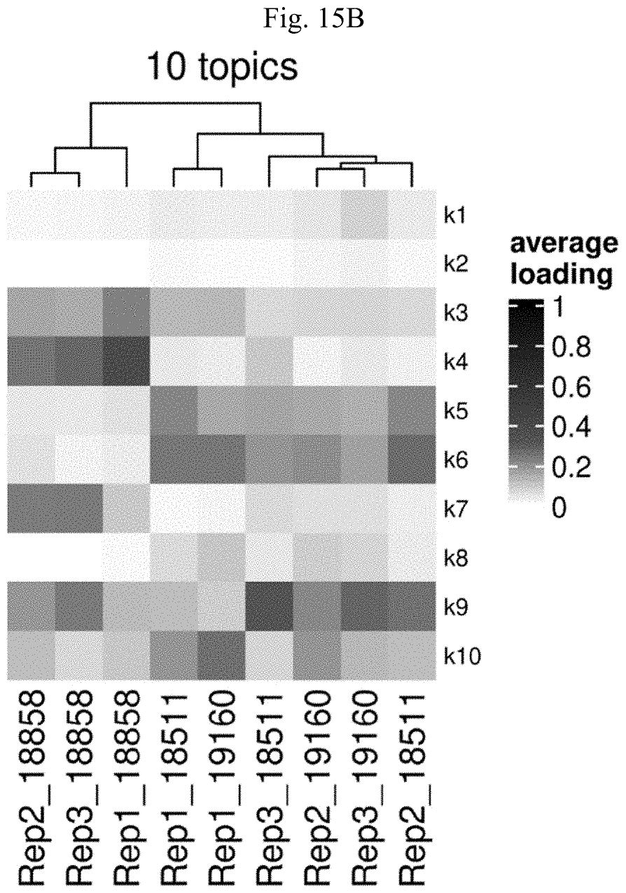

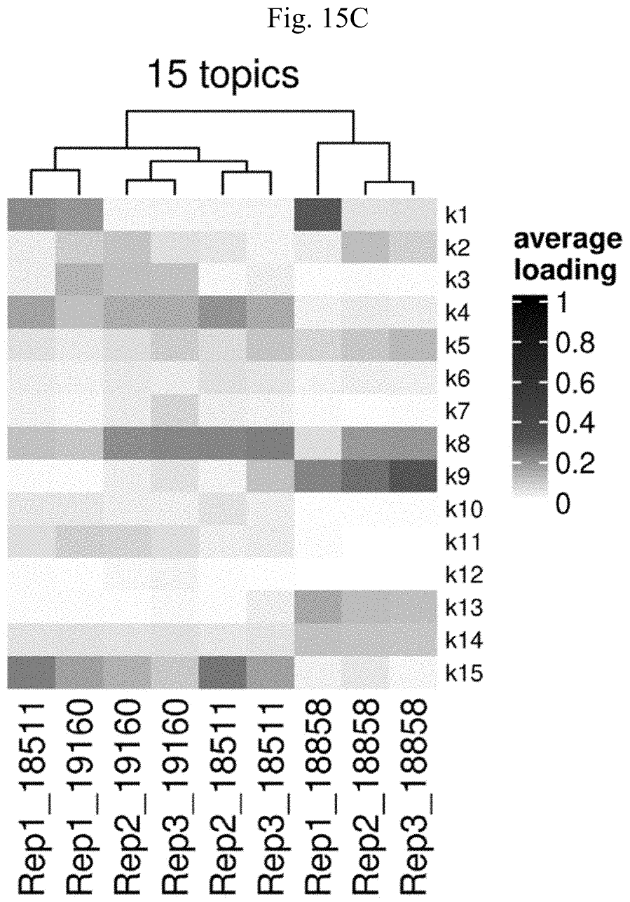

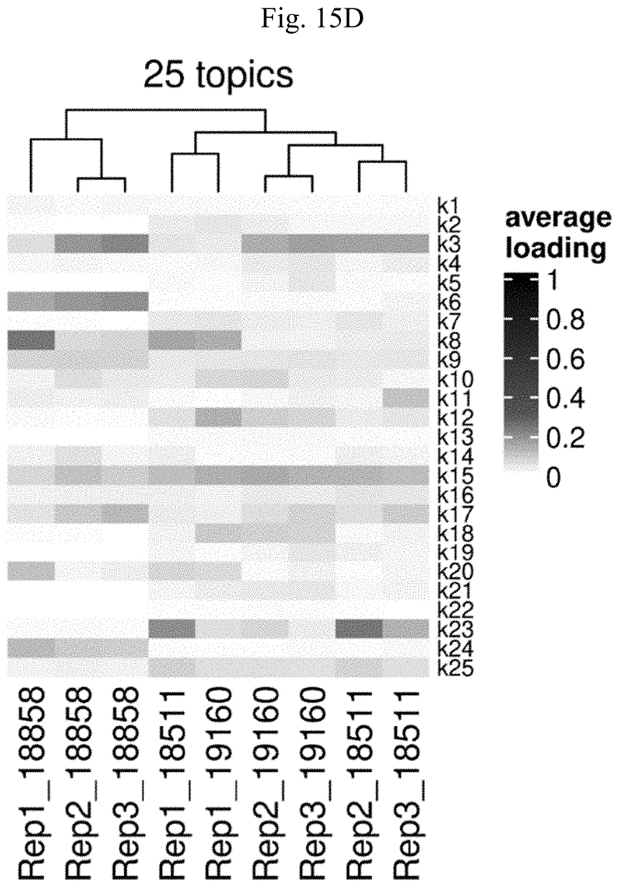

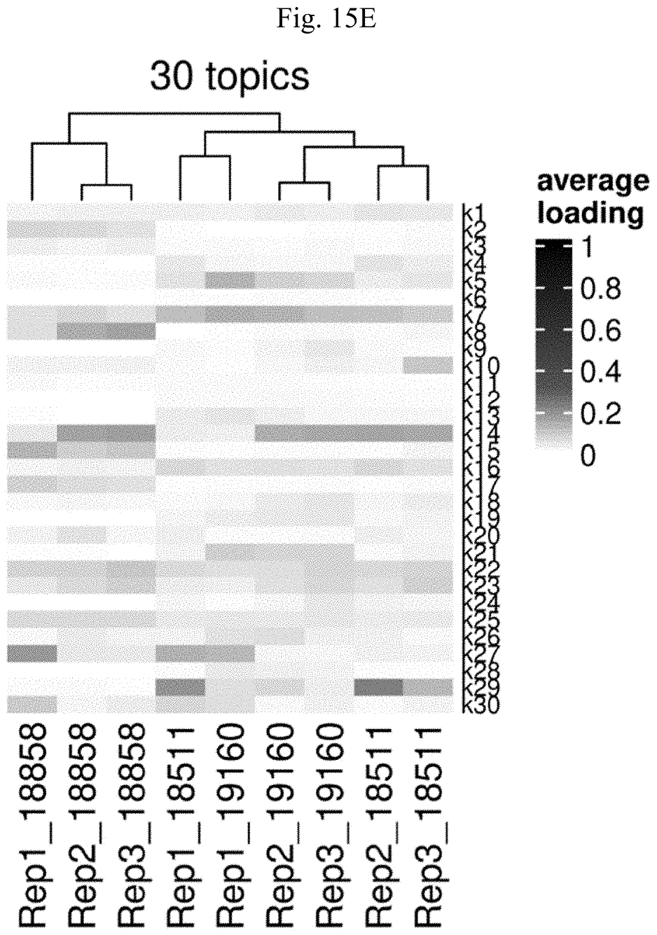

This analysis was also repeated using topic loadings as a measure of cell type composition. The loading of each topic was calculated on each individual-replicate group and hierarchical clustering was performed (FIGS. 15A, 15B, 15C, and 15D). Again, at varying values of k, samples generally cluster by individual, but using the higher resolution topic-based approach, there was substantial variation between replicates (FIGS. 15A, 15B, 15C, and 15D). Individual 18858 always clusters away from the other two lines, due to the consistent and distinct distribution of cell types caused by low differentiation efficiency.

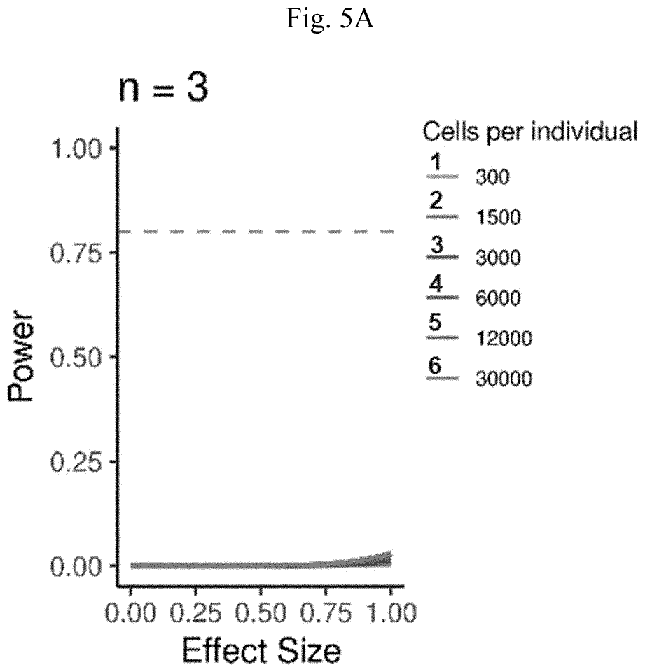

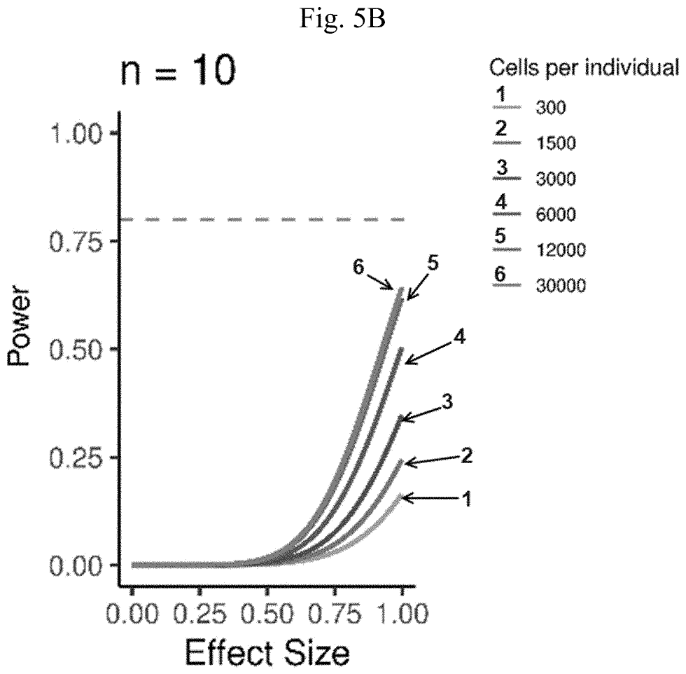

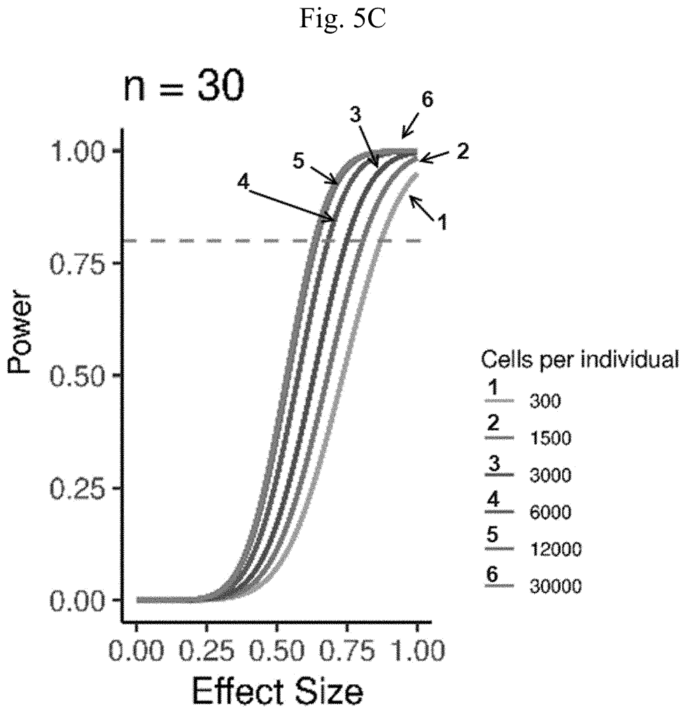

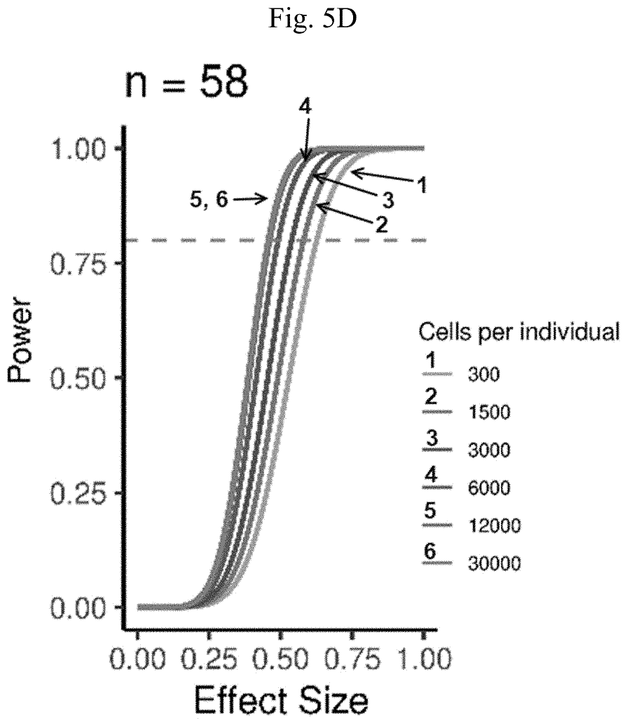

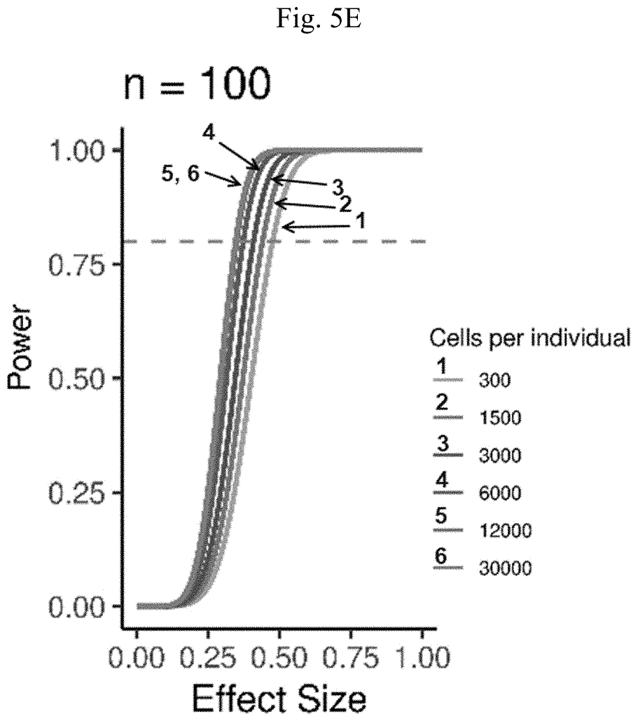

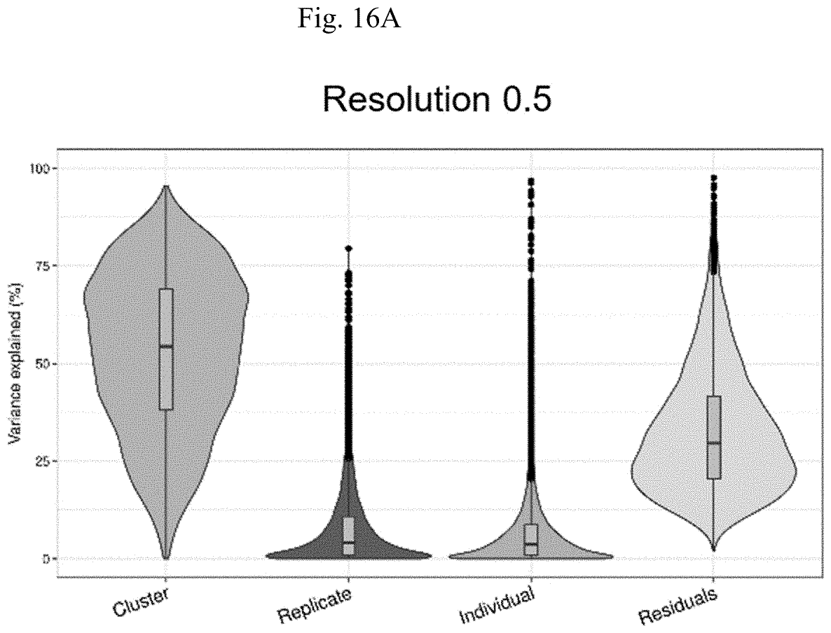

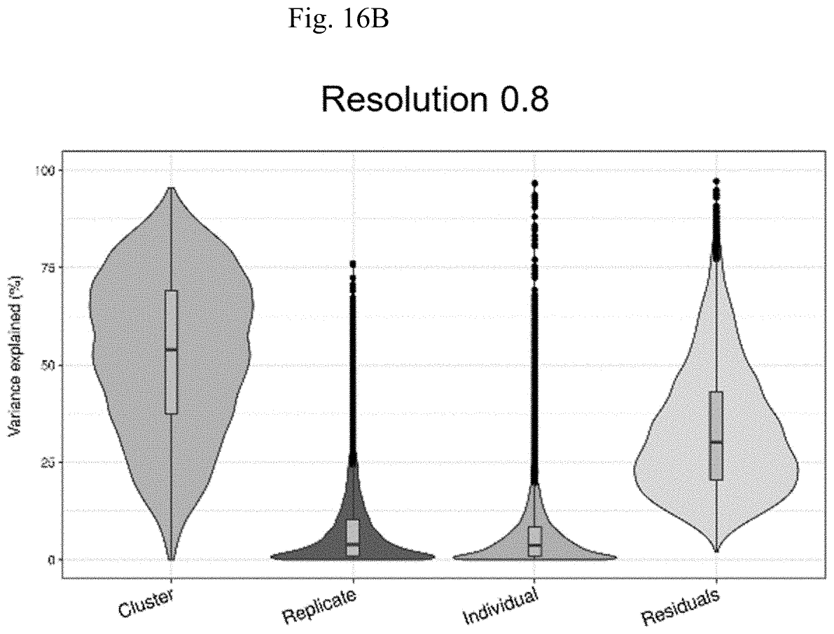

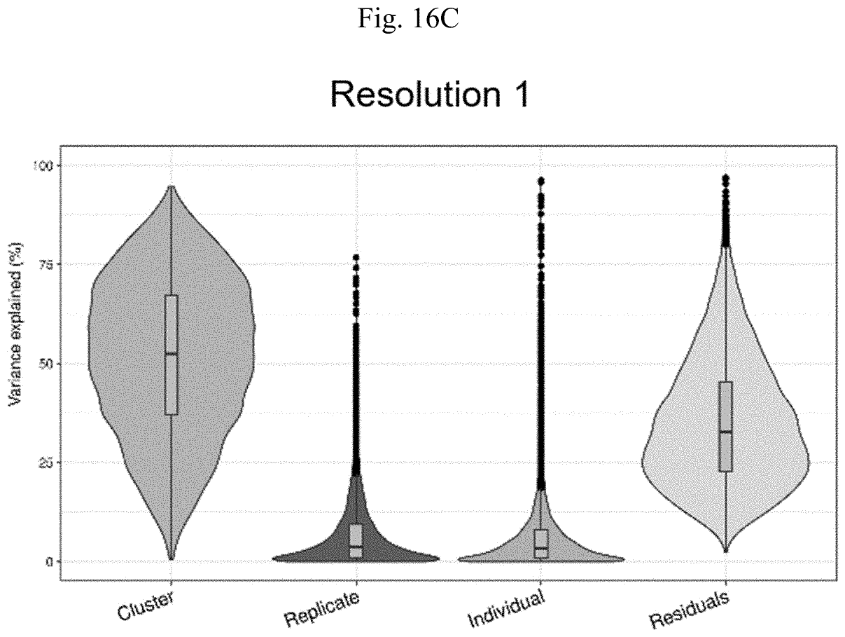

Determinants of variation were further characterized in this system by considering factors that contribute to variation in gene expression levels. Hierarchical clustering of pseudobulk expression estimates of cells from the same Seurat cluster (res. 0.1), replicate, and individual shows that, as might be expected, samples tend to cluster first by cell type (Seurat cluster), then by individual and replicate (FIG. 5A). Variance partitioning was performed using pseudobulk expression levels to estimate the relative contribution of cell type, individual, and replicate to overall patterns of gene expression variation (FIG. 5B; Hoffman and Schadt, 2016). Replicate and individual explained approximately equal proportions of the variance (each explains a median value of −5% of variance). Cell type identity explained the largest proportion of variation at all clustering resolutions tested (variance explained median value −60% at clustering resolution 0.1), although this figure is likely exaggerated since cell type identity is determined by clustering, which will inherently maximize variation between cell types. Depending on clustering resolution, a median value of approximately 20-30% of variance is explained by residuals, which can be attributed to noise or other technical variation not specifically modeled (FIGS. 16A, 16B, and 16C). The variance in gene expression was then partitioned at single cell resolution (instead of using pseudobulk estimates) and the replicate was found to explain more variation on average than individuals, with cell type identity continuing to explain more variance than either (FIG. 5C). At single-cell resolution, residuals explain a median value of 93% of the variation, which is expected due to the high degree of variance (both biological and technical) in gene expression profiles among individual cells.

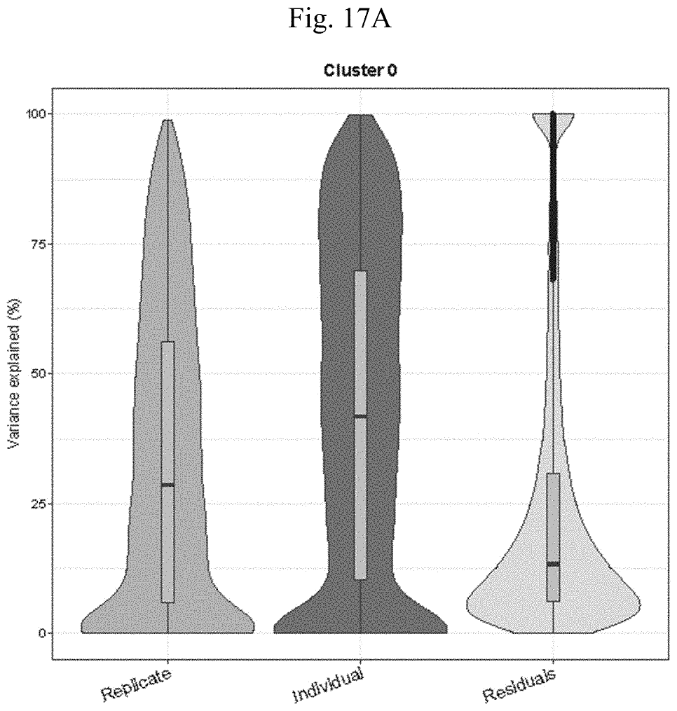

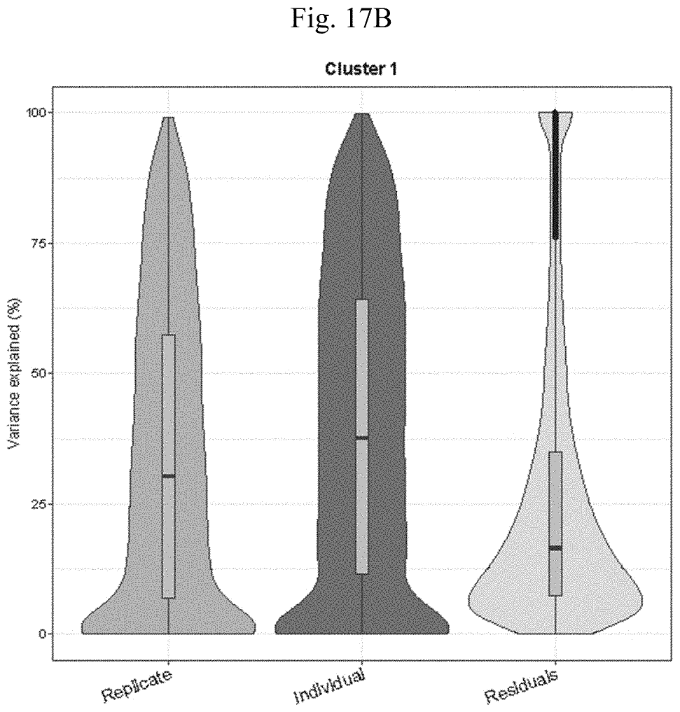

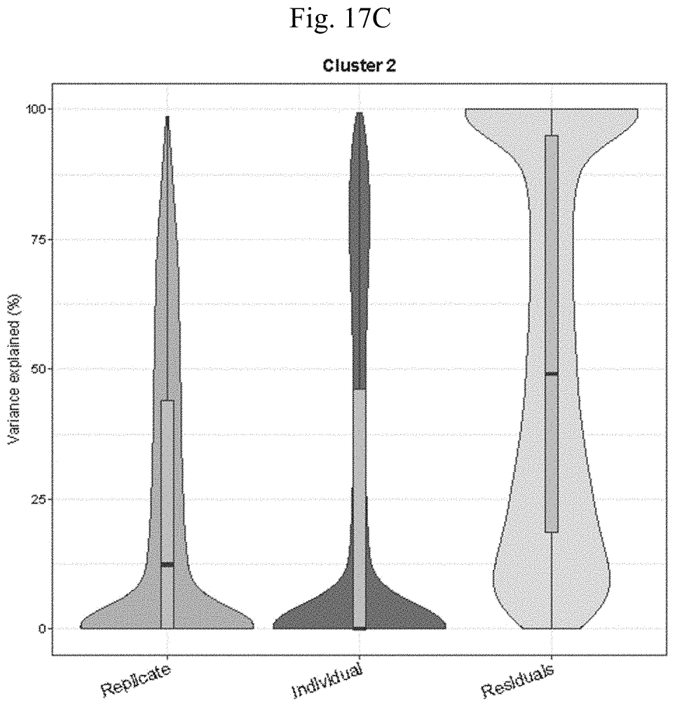

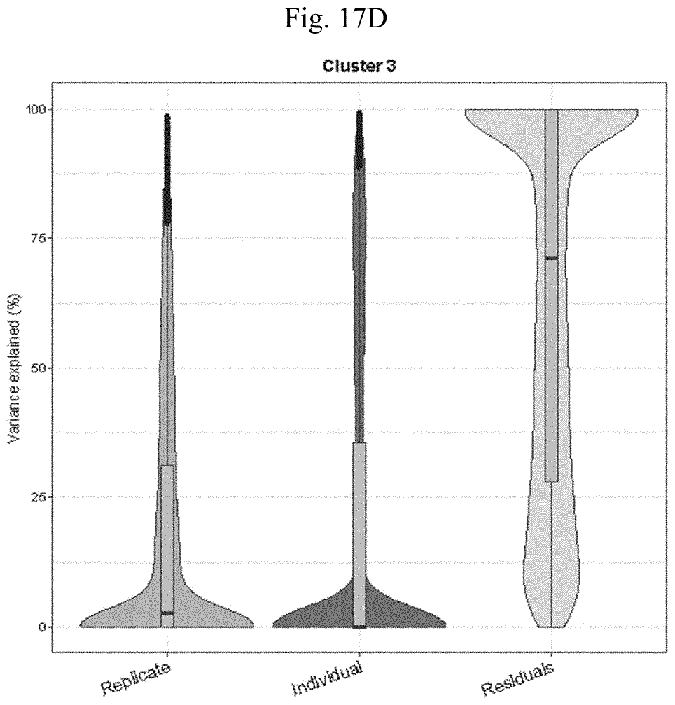

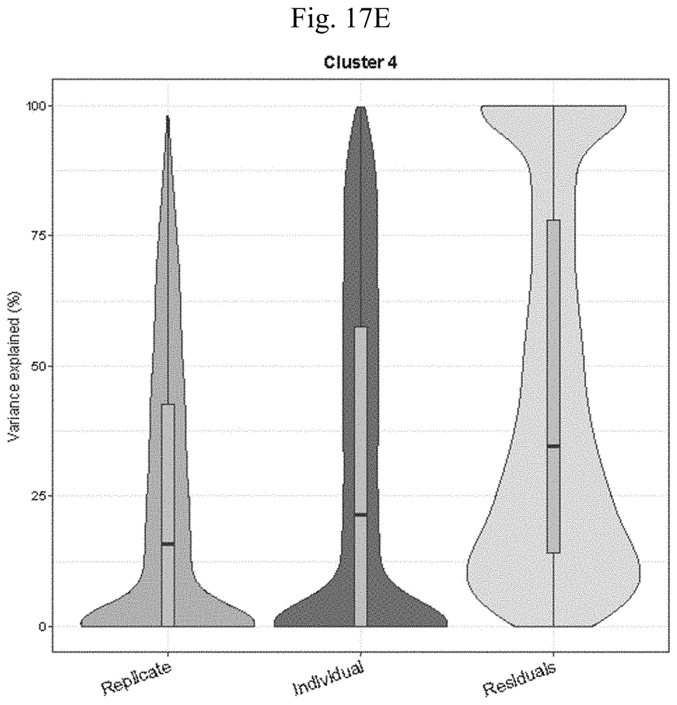

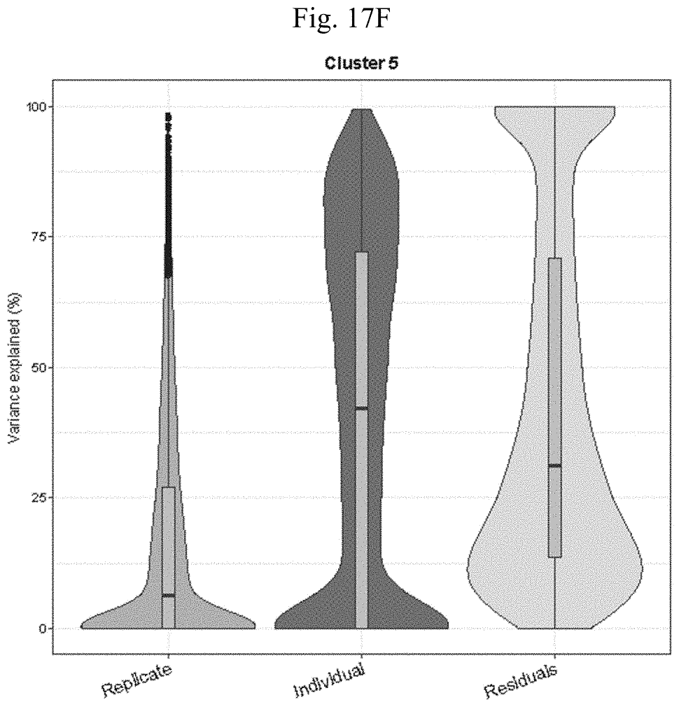

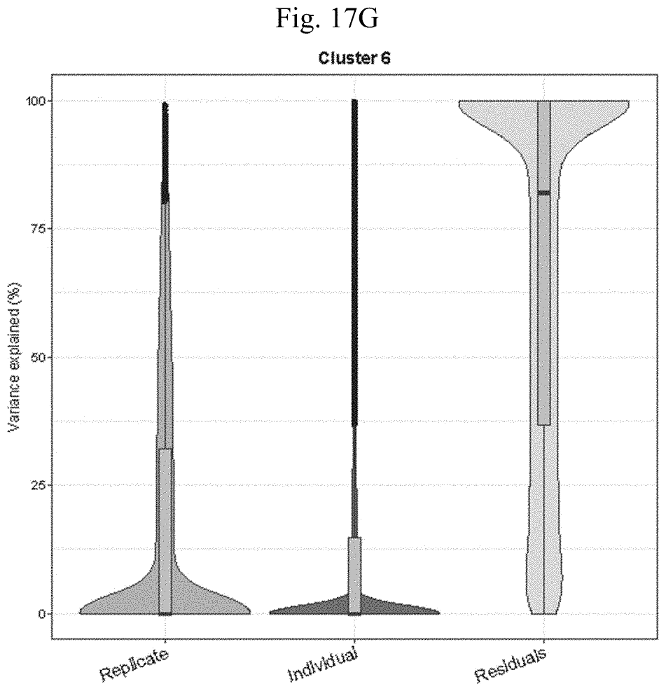

To determine whether biological and technical factors contributed differently to variation between cell types, the variance due to replicate and individual was partitioned in each Seurat cluster separately (FIGS. 17A, 17B, 17C, 17D, 17E, 17F, 17G, and 18). The results are not uniform across clusters. At clustering resolution 0.1, individual contributes more to variation, on average, in clusters 0, 1, 4, and 5, while replicate contributes more to variation in clusters 2, 3, and 6. Notably, clusters 2, 3, and 6 include only a few cells from individual 18858 (Table 4).

Because individual variation contributes to overall patterns of variation in gene expression, MTOs have the potential to be a powerful model to understand inter-individual variation in gene regulation across cell types and to map dynamic eQTLs. A power analysis was performed to better understand the relationship between power, sample size, and the total number of individual cells analyzed, or the experiment size (FIGS. 5A, 5B, 5C, 5D, and 5E). Assuming a simple linear regression to map eQTLs and a conservative Bonferroni correction for multiple testing (FWER=0.05, Materials and methods), an experiment consisting of 58 individuals with 3000 cells collected per individual, collected across three replicates (experiment size of 174,000 cells total), was estimated to provide 93% power to detect eQTLs with a standardized effect size of 0.6. These assumptions represent an experimentally tractable study design, and a conservative estimate of standard and dynamic eQTL effect sizes, suggesting this could be a powerful system for QTL studies across diverse human cell types.

### Dynamic Patterns of Gene Expression

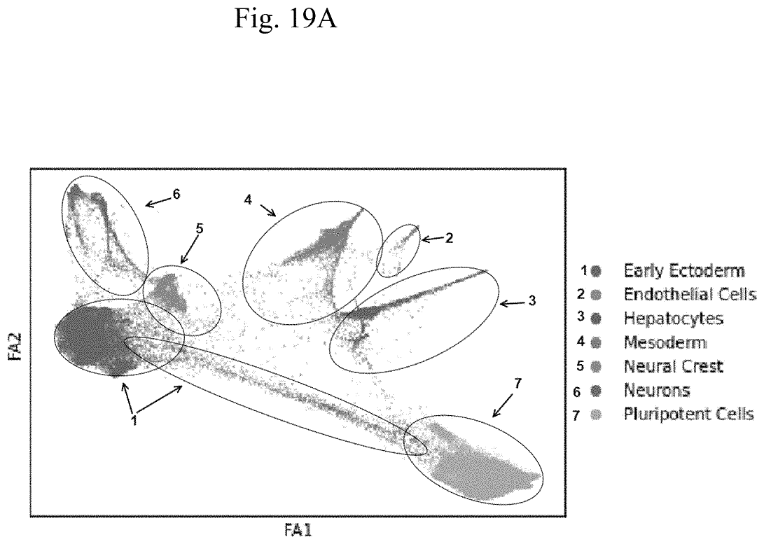

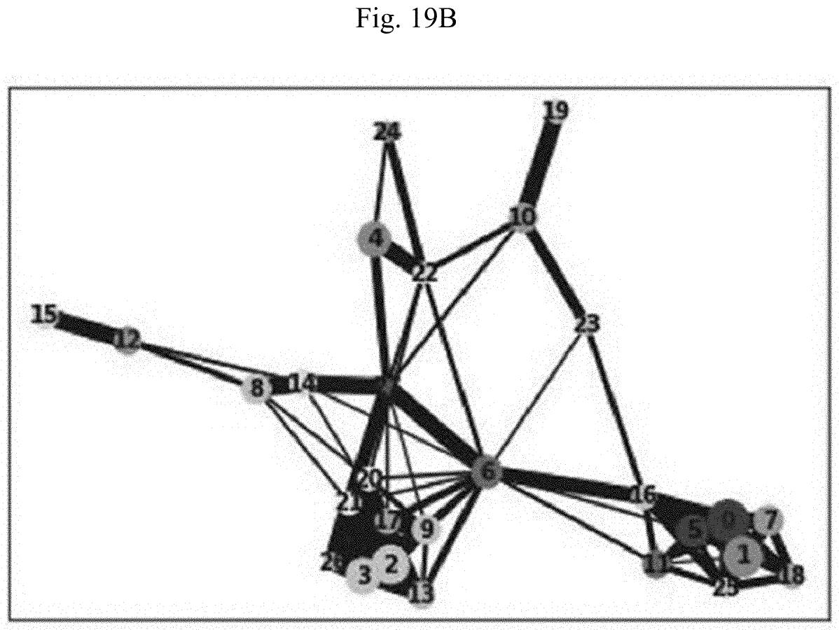

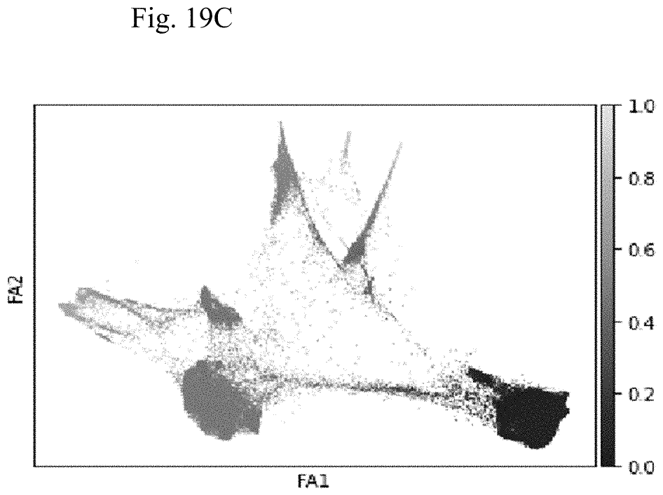

Arguably, the most attractive property of single-cell data from the MTO system is the ability to study dynamic gene regulatory patterns throughout differentiation. In order to explore dynamic patterns of gene expression, developmental trajectories were inferred using PAGA (Wolf et al., 2019). The PAGA graph shows edges that represent likely connections between cell clusters (clustering resolution 1) and developmental trajectories were traced through these paths (FIGS. 6A, 6B, 6C, 19A, and 19B). Since the MTOs still include undifferentiated pluripotent cells, rooted trajectories could be defined to each germ layer beginning at the known starting point. Trajectories toward endoderm and mesoderm proceed through cluster 22, which expresses primitive streak marker MIXL1, showing recapitulation of developmental trajectories defined during gastrulation (FIGS. 19A, 19B, and 19C). Hepatocytes (cluster 19), an endoderm-derived cell type, branch off of the endoderm cluster (cluster 10) (FIG. 6B). Endothelial cells (cluster 24), which are derived from mesoderm, branch off from the mesoderm cluster (cluster 4) (FIG. 6C), and neurons (clusters 12, 15), an ectoderm-derived cell type, branch off from the early ectoderm clusters (clusters 2, 3, 8, 9, 13, 14, 17, 20, 21, 26, and 27) (FIG. 6A and Table 2).

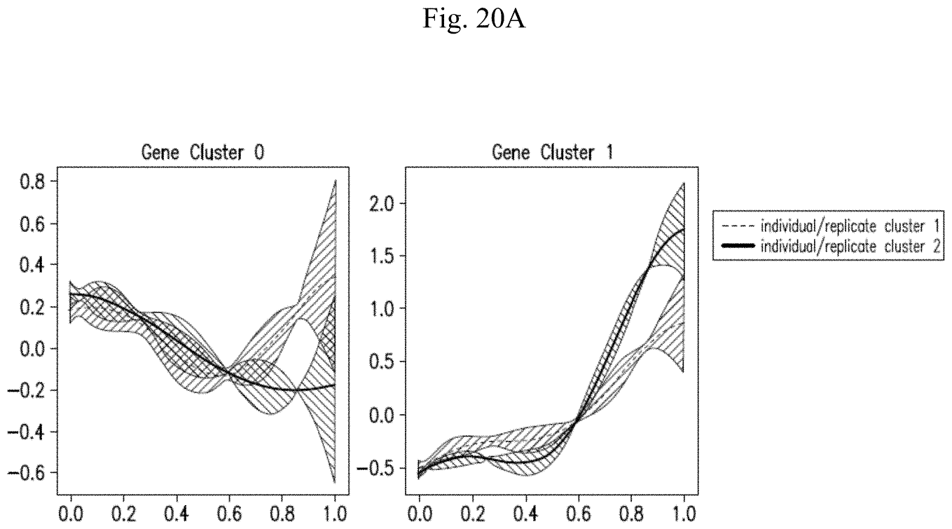

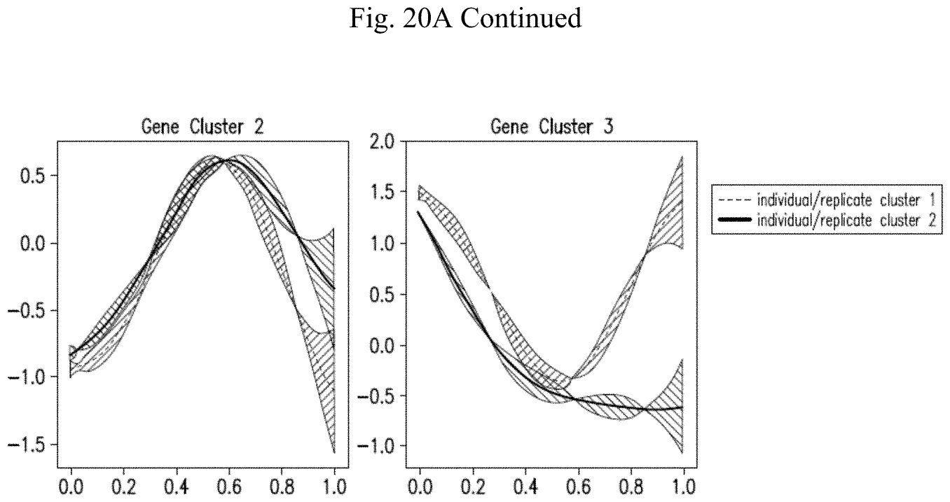

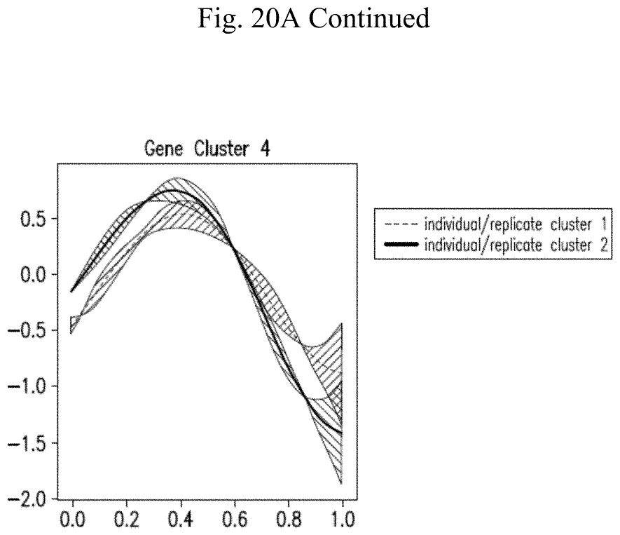

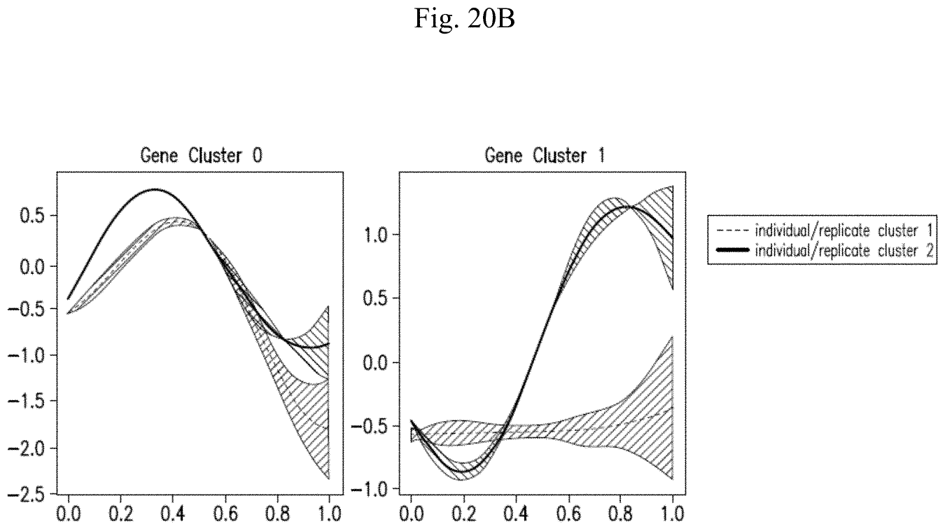

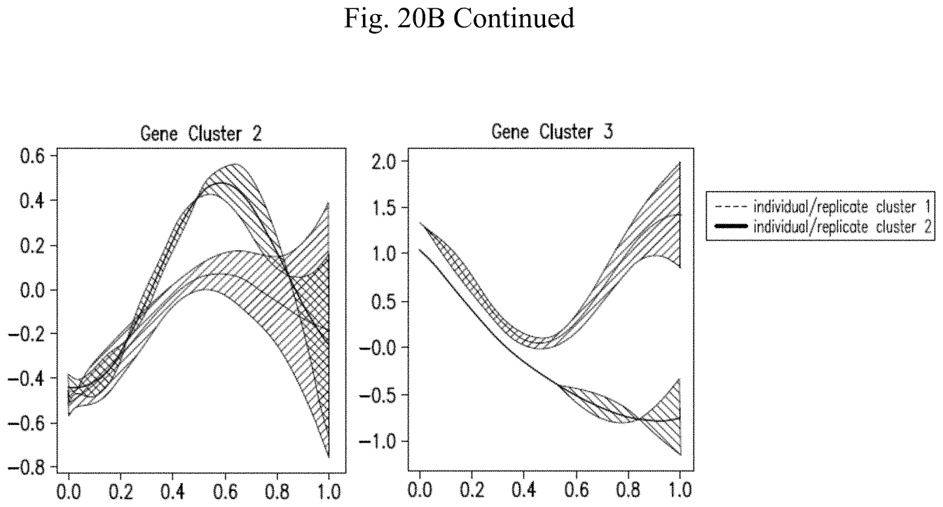

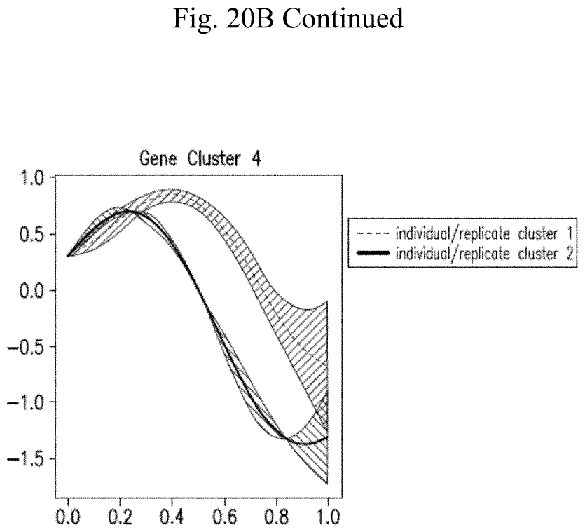

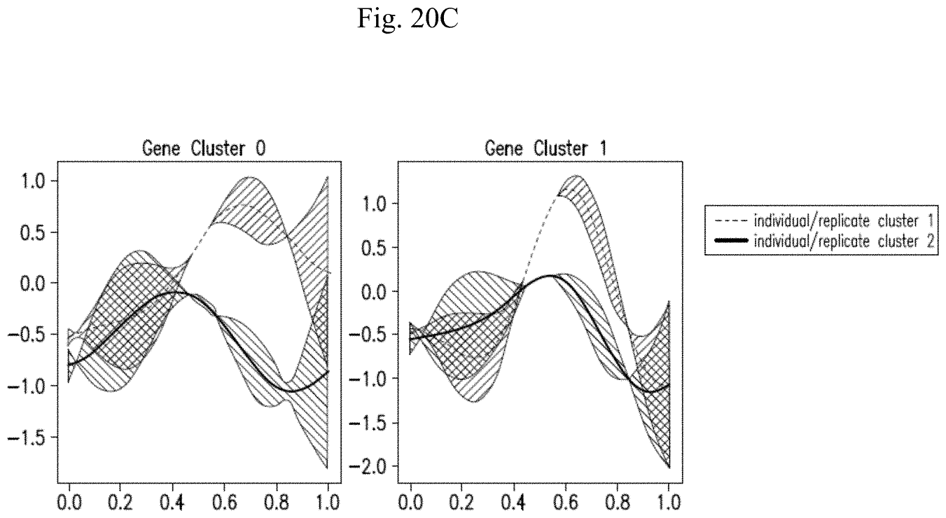

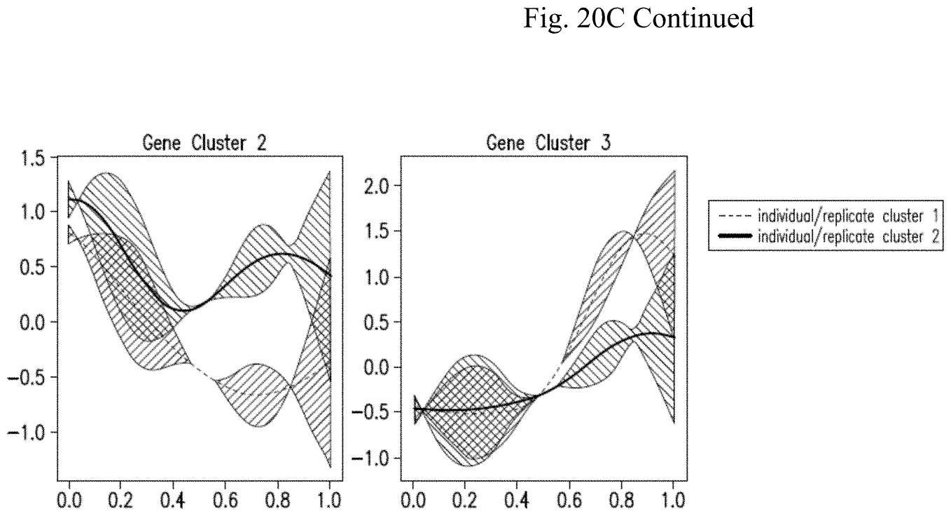

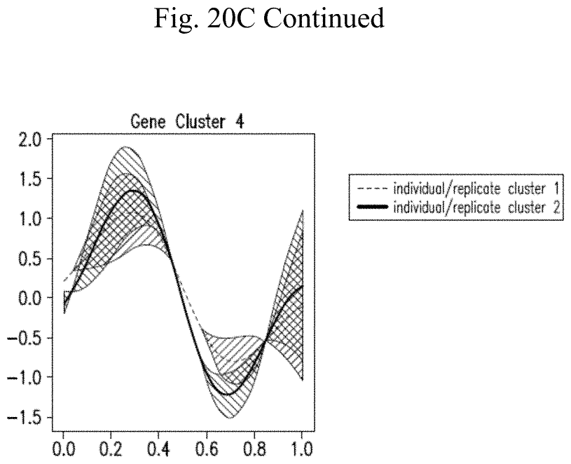

Pseudo-time values were then assigned to each cell using the diffusion pseudo-time method with pluripotent cells (cluster 1) defined as the root (Haghverdi et al., 2016; FIG. 19C). High confidence trajectories were manually traced through the data representing the progression from pluripotent cells to hepatocytes (clusters 0, 1, 5, 6, 7, 10, 11, 16, 18, 19, 25, and 22) (FIG. 6B), pluripotent cells to endothelial cells (clusters 0, 1, 4, 5, 6, 7, 11, 16, 18, 22, 24, and 25) (FIG. 6C), and pluripotent cells to neurons (clusters 0, 1, 2, 3, 5, 6, 7, 8, 9, 11, 12, 13, 14, 15, 16, 17, 18, 20, 21, 26, and 27) (FIG. 6A). For groups of clusters with a higher degree of connectivity (e.g. clusters expressing pluripotent markers and clusters expressing early ectoderm markers), all clusters within the region with high connectivity were included in the trajectory to avoid choosing an arbitrary path through these clusters. Next, split-GPM, an unsupervised probabilistic model, was applied to infer dynamic patterns of gene expression within a particular developmental trajectory, while simultaneously performing clustering of genes and samples. Split-GPM is built for use with time course, bulk RNA-seq data; therefore, pseudobulk expression values were calculated for individual-replicate groups within decile bins of pseudo-time. Gene modules with distinct dynamic patterns of expression were identified along the trajectories to neurons, hepatocytes, and endothelial cells (FIGS. 20A, 20B, 20C, and Tables 6, 7, 8, and 9).

TABLE 6
Neuronal lineage Bonferonni-adjusted p-values
from gene set enrichment

| | Cluster | Cluster | Cluster | Cluster |
|---|---|---|---|---|
| Gene Modules | 1 | 2 | 3 | 4 |
| MITOTIC SPINDLE | N/A | N/A | 1 | 2.17277E−06 |
| G2M CHECKPOINT | N/A | N/A | 1 | 1.48348E−22 |
| ESTROGEN | N/A | N/A | 0.000267846 | 1 |
| RESPONSE EARLY | | | | |
| MTORC1 SIGNALING | N/A | N/A | 6.99E−08 | 1 |
| E2F TARGETS | N/A | N/A | 1 | 3.49535E−25 |
| MYC TARGETS V1 | N/A | N/A | 0.192287 | 9.34926E−06 |
| MYC TARGETS V2 | N/A | N/A | 5.43658E−07 | 1 |
| OXIDATIVE | N/A | N/A | 0.0026008 | 1 |
| PHOSPHORYLATION | | | | |
| REACTIVE OXYGEN | N/A | N/A | 0.000274695 | 1 |
| SPECIES PATHWAY | | | | |

TABLE 7
Hepatic lineage Bonferonni-adjusted p-values from gene set enrichment

| Gene Modules | Cluster 1 | Cluster 2 | Cluster 3 | Cluster 4 |
|---|---|---|---|---|
| CHOLESTEROL | 6.92618E−09 | N/A | N/A | 1 |
| HOMEOSTASIS | | | | |
| MITOTIC SPINDLE | 1 | N/A | N/A | 4.92256E−13 |
| G2M CHECKPOINT | 1 | N/A | N/A | 1.17837E−27 |
| ADIPOGENESIS | 4.37992E−05 | N/A | N/A | 1 |
| COMPLEMENT | 7.02289E−06 | N/A | N/A | 1 |
| E2F TARGETS | 1 | N/A | N/A | 2.31674E−33 |
| MYC TARGETS V1 | 1 | N/A | N/A | 6.21581E−10 |
| XENOBIOTIC | 4.90832E−10 | N/A | N/A | 1 |
| METABOLISM | | | | |
| FATTY ACID | 4.23928E−09 | N/A | N/A | 1 |
| METABOLISM | | | | |
| COAGULATION | 8.51378E−14 | N/A | N/A | 1 |
| BILE ACID | 2.19004E−06 | N/A | N/A | 1 |
| METABOLISM | | | | |
| PEROXISOME | 0.000565521 | N/A | N/A | 1 |

TABLE 8
Epithelial lineage Bonferonni-adjusted p-values from gene set enrichment

| Gene Modules | Cluster 1 | Cluster 2 | Cluster 3 | Cluster 4 |
|---|---|---|---|---|
| TNFA SIGNALING | 1 | 1 | 4.10533E-07 | 1 |
| VIA NFKB | | | | |
| G2M CHECKPOINT | 1 | 1 | 1 | 8.08289E−08 |
| APOPTOSIS | 1 | 1 | 0.000118488 | 1 |
| APICAL JUNCTION | 1 | 1 | 0.000755343 | 1 |
| COMPLEMENT | 1 | 1 | 6.53390E−06 | 1 |
| MTORC1 SIGNALING | 1 | 1.78151E−05 | 1 | 1 |
| E2F TARGETS | 1 | 1 | 1 | 3.35097E−13 |
| MYC TARGETS V2 | 1 | 0.000987191 | 1 | 1 |
| EPITHELIAL | 6.02819E−05 | 1 | 1 | 1 |
| MESENCHYMAL | | | | |
| TRANSITION | | | | |
| INFLAMMATORY | 1 | 1 | 0.00462036 | 1 |
| RESPONSE | | | | |
| OXIDATIVE | 1 | 0.00108741 | 1 | 1 |
| PHOSPHORYLATION | | | | |
| COAGULATION | 1 | 1 | 0.000313316 | 1 |
| IL2 STAT5 | 1 | 1 | 0.00202719 | 1 |
| SIGNALING | | | | |

TABLE 9
splitGPM cluster assignments of each individual-batch sample
based on shared patterns of dynamic gene expression.

| Cell Lineage | | Cell Line & Batch | Assignment |
|---|---|---|---|
| Neuronal | 0 | SNG-NA 18511 Batch 1 | Mesoderm |
| Neuronal | 1 | SNG-NA 18511 Batch 2 | Mesoderm |
| Neuronal | 2 | SNG-NA 18511 Batch 3 | Mesoderm |
| Neuronal | 3 | SNG-NA 18858 Batch 1 | Early Ectoderm |
| Neuronal | 4 | SNG-NA 18858 Batch 2 | Mesoderm |
| Neuronal | 5 | SNG-NA 18858 Batch 3 | Early Ectoderm |
| Neuronal | 6 | SNG-NA 19160 Batch 1 | Mesoderm |
| Neuronal | 7 | SNG-NA 19160 Batch 2 | Mesoderm |
| Neuronal | 8 | SNG-NA 19160 Batch 3 | Mesoderm |
| Hepatic | 0 | SNG-NA 18511 Batch 1 | Mesoderm |
| Hepatic | 1 | SNG-NA 18511 Batch 2 | Mesoderm |
| Hepatic | 2 | SNG-NA 18511 Batch 3 | Mesoderm |
| Hepatic | 3 | SNG-NA 18858 Batch 1 | Mesoderm |
| Hepatic | 4 | SNG-NA 18858 Batch 2 | Early Ectoderm |
| Hepatic | 5 | SNG-NA 18858 Batch 3 | Early Ectoderm |
| Hepatic | 6 | SNG-NA 19160 Batch 1 | Mesoderm |
| Hepatic | 7 | SNG-NA 19160 Batch 2 | Mesoderm |
| Hepatic | 8 | SNG-NA 19160 Batch 3 | Mesoderm |
| Endothelial | 0 | SNG-NA 18511 Batch 1 | Pluripotent Cells |
| Endothelial | 1 | SNG-NA 18511 Batch 2 | Pluripotent Cells |
| Endothelial | 2 | SNG-NA 18511 Batch 3 | Pluripotent Cells |
| Endothelial | 3 | SNG-NA 18858 Batch 1 | Mesoderm |
| Endothelial | 4 | SNG-NA 18858 Batch 2 | Mesoderm |
| Endothelial | 5 | SNG-NA 18858 Batch 3 | Mesoderm |
| Endothelial | 6 | SNG-NA 19160 Batch 1 | Pluripotent Cells |
| Endothelial | 7 | SNG-NA 19160 Batch 2 | Pluripotent Cells |
| Endothelial | 8 | SNG-NA 19160 Batch 3 | Pluripotent Cells |

Gene set enrichment analysis of these modules shows expected dynamic patterns (FIGS. 20A, 20B, 20C and Tables 6, 7, 8, and 9). For example, gene modules that increase expression through pseudo-time along the differentiation trajectory to hepatocytes, which are the predominant cell type of the liver and are responsible for the production of bile, are enriched for the hallmark bile acid metabolism and fatty acid metabolism gene sets. In the trajectory leading to endothelial cells, which are derived from mesoderm, a gene module with high expression at intermediate pseudo-time values is enriched for hallmark genes expressed during the epithelial-mesenchymal transition, which is important for mesoderm formation (Evseenko et al., 2010). In all three trajectories, gene modules characterized with higher expression at low pseudo-time values show enrichment for gene sets related to the cell cycle; this is expected because pluripotent cells at the lowest pseudo-time values tend to grow and divide faster than more differentiated and more mature cell types, which often exit the cell cycle (Buttitta and Edgar, 2007).

To determine the consistency in dynamic patterns of gene expression between replicates and individuals, split-GPM was run ten times on cells from the neuron, hepatocyte, and endothelial cell lineages and each pair of individual-replicate samples were observed to measure how often they clustered together (FIGS. 6D, 6E, and 6F). In the neuronal and endothelial lineages, all three replicates of 18511 always clustered together and often cluster with replicates of 19160, indicating that these two lines share similar expression dynamics in these trajectories (FIGS. 6D and 6F). Replicates of 18858 often clustered together and rarely clustered with the other individuals, suggesting that not only did 18858 have poor differentiation efficiency, but cells that did differentiate show a distinct pattern of expression dynamics. In the hepatocyte lineage, stronger replicate-specific differences were observed (FIG. 6E). Replicates of individual 19160 still tended to cluster together and to cluster with replicates 1 and 2 of 18511. Replicate 3 of 18511 rarely clustered with the other replicates of that individual, indicating that there were replicate-specific effects on dynamic gene expression.

### Differential Expression Between Humans and Chimpanzees in 72 Cell Types

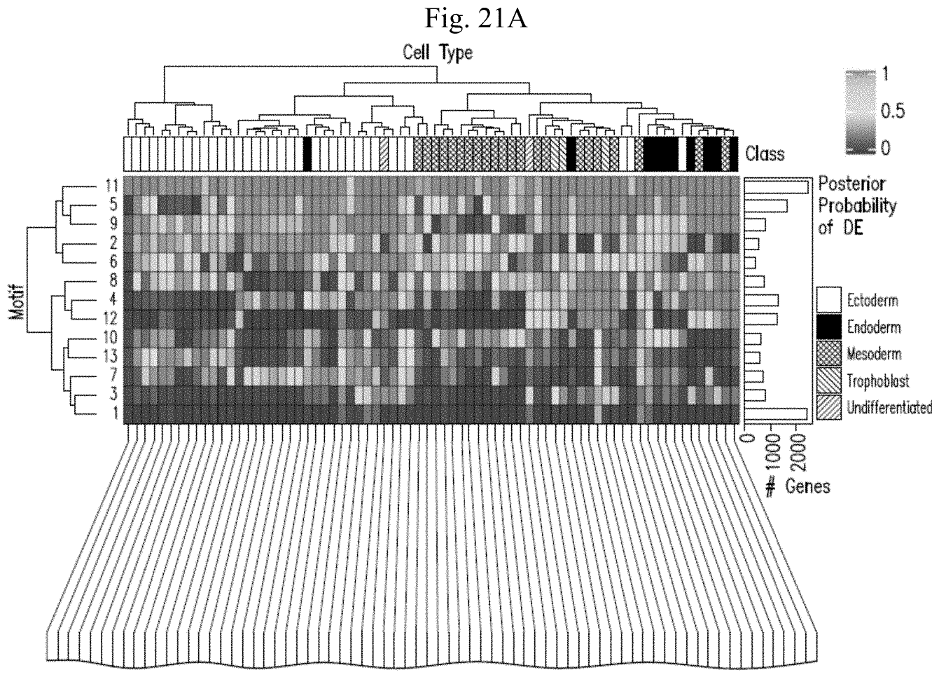

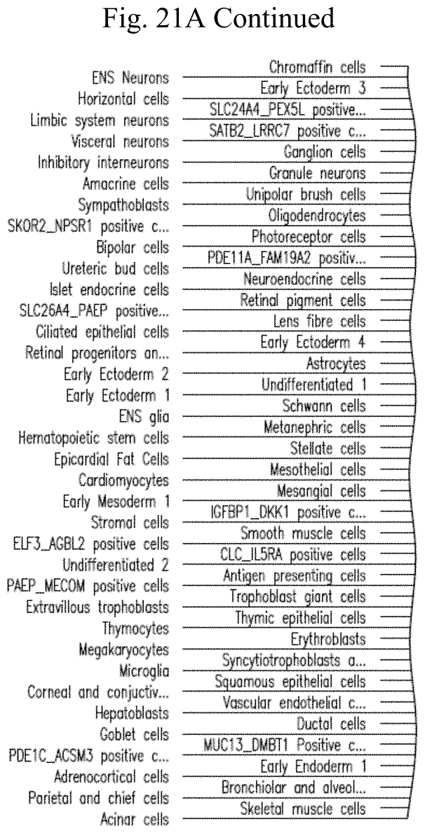

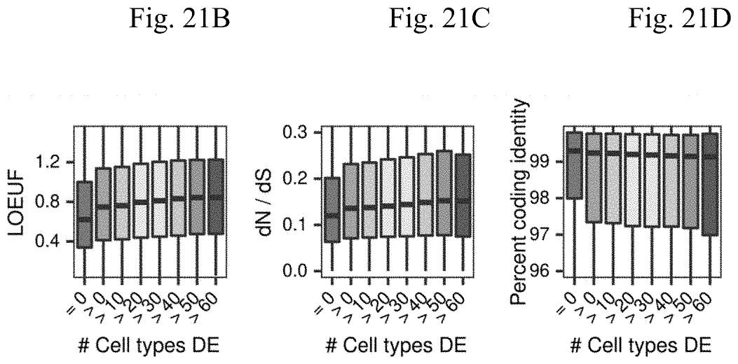

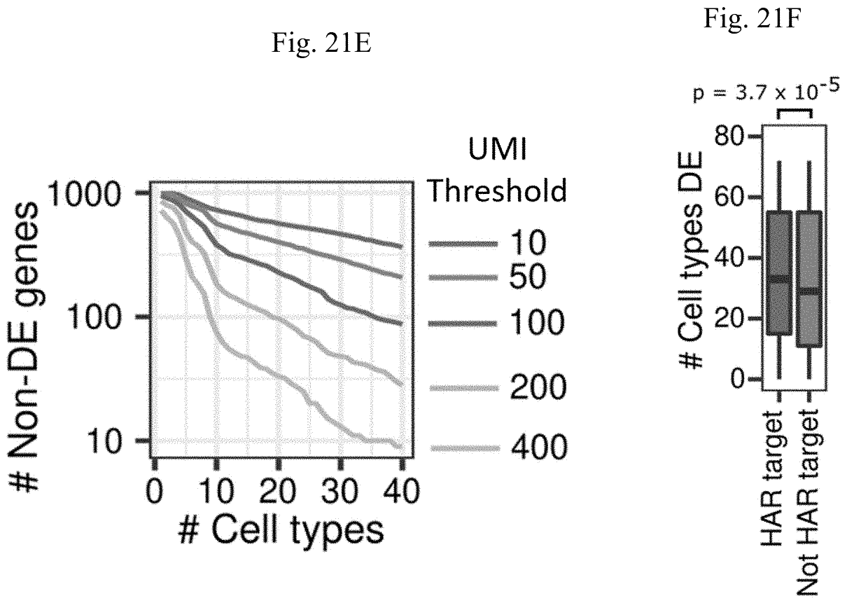

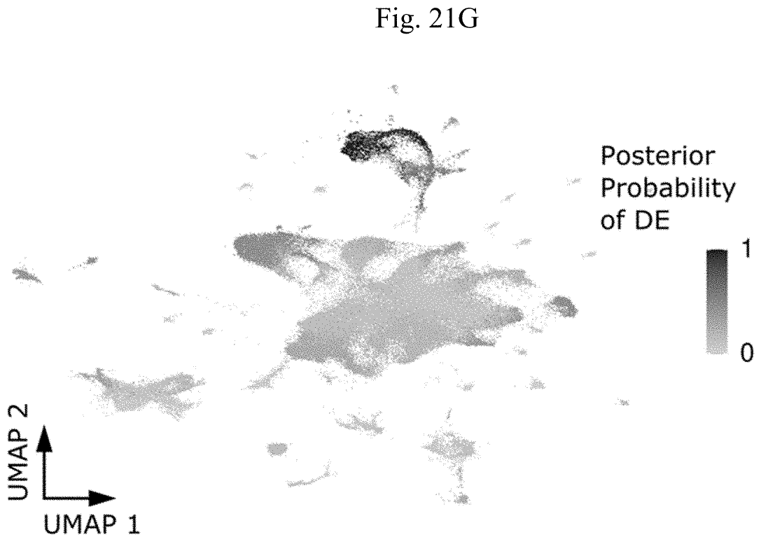

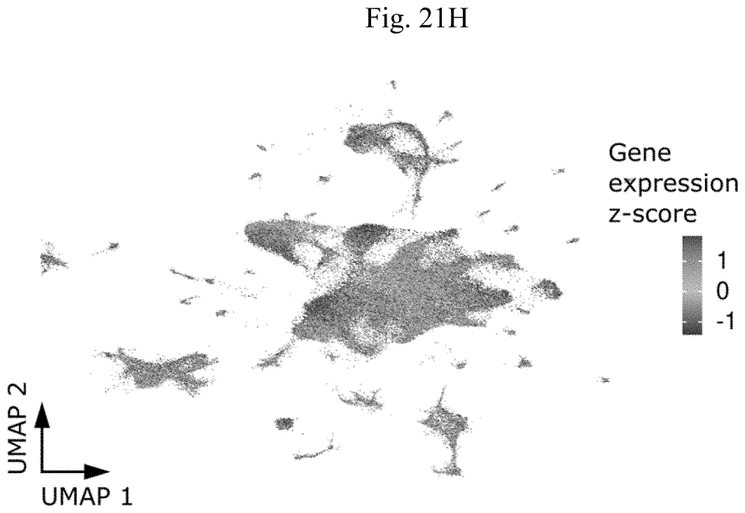

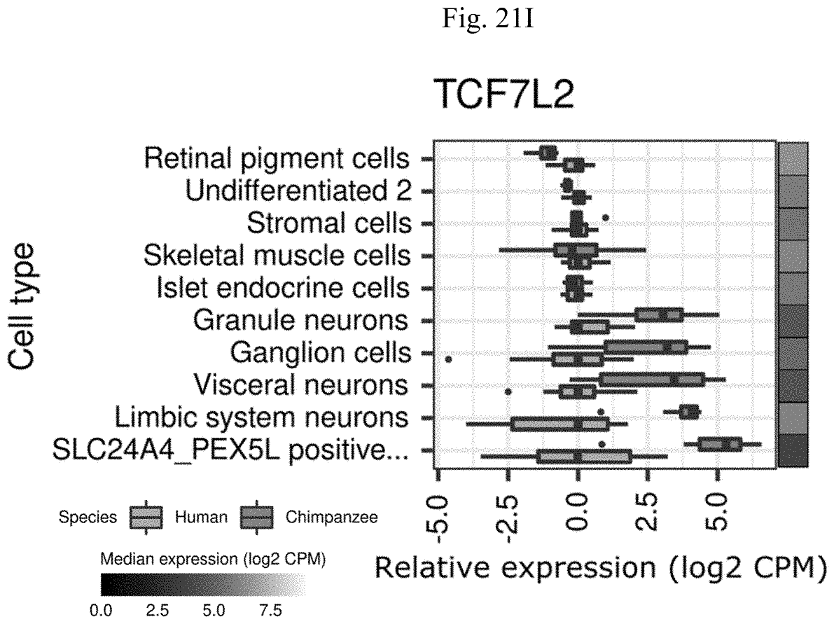

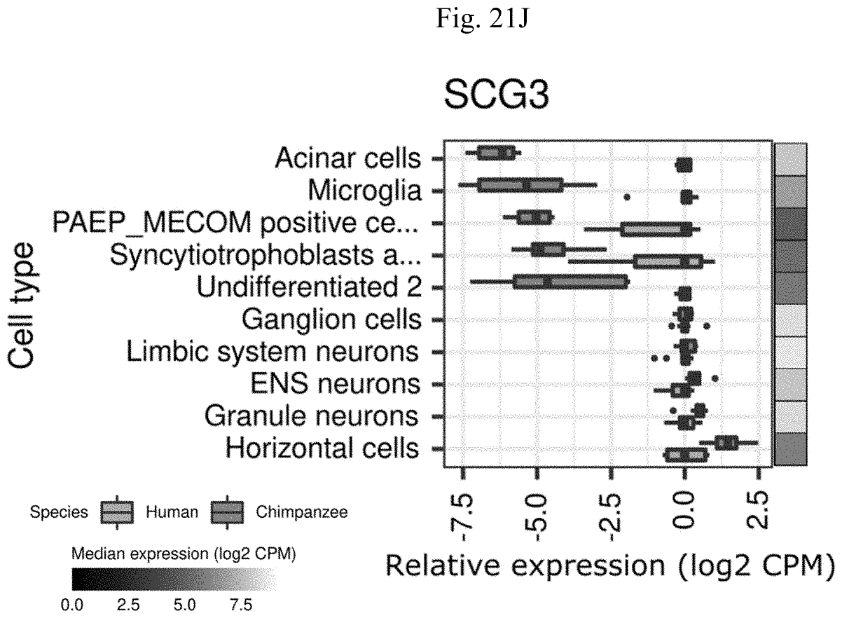

Differential expression (DE) was characterized between humans and chimpanzees in individual cell types using similar methods as above. Examining the results (FIG. 21A), the two largest classes of genes were found to be those that are DE between species in all cell types (motif 11) and those that are not DE in any cell type (motif 1). These patterns of DE could indicate different degrees of selective constraint. Indeed, the number of cell types in which a gene is classified as DE between species were found to be significantly associated with coding sequence similarity between humans and chimpanzees (FIG. 21B; p=2.7×10-6). The ratio of non-synonymous to synonymous coding mutations across mammals (dN/dS) and the loss-of-function observed/expected upper bound fraction (LOEUF) (Karczewski et al., 2020) are also positively associated with the number of cell types in which a gene is classified as DE (FIG. 21C-21D; dN/dS p=10-13; LOEUF p<10-15). These observations are consistent with the notion that broad gene expression differences between humans and chimpanzees are generally associated with relaxation of evolutionary constraint. For a subset of broadly DE genes, it may be the case that directional selection has favored a new regulatory pattern. In support of this, we found that genes targeted by human accelerated regions (HARs) are also DE in a greater number of cell types (FIG. 21E; p=4.6×10-5).

Genes with conserved expression levels across all cell types are of particular interest, as they may shed insight on core functions in both species. Yet, the identification of these genes is confounded by statistical power. To confidently classify these genes, non-DE genes were filtered by their absolute expression levels (FIG. 21F). Hundreds of non-DE genes were classified across all cell types, even at relatively stringent filters. For example, non-DE genes with at least 100 UMIs in at least 10 cell types in both species are enriched for core cellular processes, including protein transport and mRNA processing, splicing, and transport.

Beyond genes that are DE in no cell types or all cell types, many of the remaining DE motifs have tissue-specific patterns. Inter-species DE in specific tissues could be driven by tissue-specific expression patterns, where genes may be DE everywhere they are expressed. In other cases, a gene may have acquired DE in a restricted set of cell types, despite being expressed in additional cell types. To examine these possibilities, the expression levels of the genes in each motif were considered across all cell types and compared to patterns of DE (FIGS. 21G and 21H). Motifs exhibiting tissue-specific DE while the genes are highly expressed, but are conserved in other cell types were focused on. Genes with cell type-restricted acquisition of DE were then identified. 3,906 genes (27%) were identified that are broadly expressed but show cell-type-specific DE between humans and chimpanzees.

Tissue-restricted DE could underlie important functional differences between humans and chimpanzees. For instance, the transcription factor (TF) TCF7L2 is a WNT modulator associated with human psychiatric and metabolic disorders (del Bosque-Plata et al., 2022, p. 7). TCF7L2 is broadly expressed in EBs, but inter-species DE is restricted to neuronal subtypes, where it may underlie inter-species differences in neurodevelopment (FIG. 21I). Another example is SCG3, an obesity-associated gene (Tanabe et al., 2007) for which inter-species DE is restricted to a few cell types, including acinar (pancreatic) cells (FIG. 21J). This cell-type-specific pattern could underlie inter-species differences in digestive activity.

### Discussion

This work represents an exploration of heterogeneity in single cell data obtained from human MTOs. iPSC-derived MTOs were used because this in vitro model system circumvents the logistical challenges and ethical barriers associated with studies of primary human developmental tissues. This system has advantages over studies of primary tissues; for example, the ability to control cellular environment in vitro and intentionally design experiments with respect to biological factors including age, sex, and ancestry. Further, MTOs can be generated comprised of the same set of diverse cell types from large samples of individuals, enabling high-powered comparisons of cell-type-specific gene expression.

MTOs can be leveraged to identify QTLs and dynamic QTLs across diverse terminal and differentiating cell types. This, of course, raises a question: to what extent do the cell types derived from MTOs faithfully model immature, developing cells in vivo? MTO cells were found to express known cell-type-specific marker genes, including markers of known developmental stages. MTO cells also cluster with more than 60 diverse primary cell types from a reference panel of fetal tissues and hESCs, including rare fetal cell types. Lastly, gene modules were identified with dynamic expression patterns that match broad expectations of developmental biology. Together, these results provide evidence that MTOs are a suitable model of both terminal and developmental cell types.

Moreover, MTOs may be a useful model for understanding the genetic underpinnings of human traits and diseases regardless of the degree to which they faithfully model human development. MTO-derived cells represent a wealth of previously unstudied cell states and dynamic processes. Hypothetically, QTLs identified in these cell types still represent biologically meaningful differences in genetic control of gene regulation, whether they manifest in human development or in adult tissues upon a particular environmental exposure. Previously collected data from an in vitro differentiation experiment was considered. The 28 middle-dynamic eQTLs Strober et al. identified during the differentiation of iPSCs to cardiomyocytes (Strober et al., 2019) were examined more closely. Middle-dynamic eQTLs have their strongest genetic effects at intermediate stages of the differentiation time course, and most of them ( 25/28) were identified exclusively at these intermediate stages of differentiation. Accordingly, these eQTLs are active in early in vitro differentiating cells whose fidelity to primary developing cell types has not been ascertained. These 28 dynamic eQTLs were entirely novel and had not been identified as cis eQTLs in any tissue in the GTEx data set. Strober et al. reported that one of these middle-dynamic eQTLs was also found to overlap a GWAS variant associated with body mass index and red blood cell count. This finding highlights that dynamic eQTLs acting in early cell types in in vitro differentiations may affect long-term disease risk in adults.

To further explore the utility of dynamic eQTLs identified in in vitro differentiations, GTEx data was used to ask whether the middle-dynamic eQTLs are associated with inter-individual variation in trans gene expression or cell composition, either of which could indicate lasting downstream effects in adult tissue from transient dynamic cis eQTLs. Trans eQTL associations are more tissue-specific than cis eQTLs, but trans eQTLs are much harder to identify because of their small effect sizes and the requirement to test the association of every locus with every gene. Here, a middle dynamic eQTL SNP (rs6700162) was identified from Strober et al. that, in GTEx data, is associated with fibroblast cell type proportions in HLV (heart left ventricle; p<0.0009) and with cardiac muscle cell proportions in HLV (p<0.003). This SNP was also found to have a trans eQTL p-value of 1×10-5 in coronary artery. Without the prior knowledge provided by dynamic eQTL data from the in vitro differentiated cardiomyocytes, it would have been difficult to identify these associations using adult primary tissues because the burden of multiple testing within the entire GTEx data set is considered prohibitively large. This example implies that developing MTO cells could be used to understand how transient effects on gene expression are propagated into functional, long-lasting consequences downstream.

In this study, foundational analyses were performed to better understand how to appropriately conceptualize heterogeneity in this kind of data. Cell type composition of MTOs were explored in three paradigms; first, with discrete cell types identified with a traditional clustering algorithm, then with more continuous cell “types” identified with topic modeling, and exploring dynamic changes in gene expression along trajectories using pseudotime. Cell types defined by discrete clustering are often easier to interpret because they can be contextualized with marker genes and reference data sets defined with bulk sequencing. Overall, this study has laid the groundwork to transform MTOs into a powerful model system for the understanding of human gene regulation.

### REFERENCES

Aguet F, Brown A, Castel S, Davis J, Battle A, Brown C D, Engelhardt B E, Montgomery S B. 2017. Genetic effects on gene expression across human tissues. Nature 550:204-213. Albert F W, Kruglyak L. 2015. The role of regulatory variation in complex traits and disease.
Nature Reviews Genetics 16:197-212. Auton A, Brooks L D, Durbin R M, Garrison E P, Kang H M, Korbel J O, Marchini J L,
McCarthy S, McVean G A, Abecasis G R. 2015. A global reference for human genetic variation. Nature 526:68-74.
Banovich N E, Li Y I, Raj A, Ward M C, Greenside P, Calderon D, Tung P Y. 2018. Impact of Regulatory Variation across Human IPSCs and Differentiated Cells. Genome Research 28:122-131.
Becht E, McInnes L, Healy J, Dutertre C A, Kwok IWH, Ng L G, Ginhoux F, Newell E W. 2018. Dimensionality reduction for visualizing single-cell data using UMAP. Nature Biotechnology 37:38-44.
Belmont J W, Hardenbol P, Willis T D, Yu F, Yang H, Ch′Ang L Y, Huang W. 2003. The International HapMap Project. Nature 426:789-796.
Blondel V D, Guillaume J L, Lambiotte R, Lefebvre E. 2008. Fast unfolding of communities in large networks. Journal of Statistical Mechanics 2008:10008.
Butler A, Hoffman P, Smibert P, Papalexi E, Satija R. 2018. Integrating single-cell transcriptomic data across different conditions, technologies, and species. Nature Biotechnology 36:411-420.
Buttitta L A, Edgar B A. 2007. Mechanisms controlling cell cycle exit upon terminal differentiation. Current Opinion in Cell Biology 19:697-704.
Cao J, O'Day D R, Pliner H A, Kingsley P D, Deng M, Daza R M, Zager M A, Aldinger K A, Blecher-Gonen R, Zhang F, Spielmann M, Palis J, Doherty D, Steemers F J, Glass I A, Trapnell C, Shendure J. 2020. A human cell atlas of fetal gene expression. Science 370:eaba7721.
Cortal, A., Martignetti, L., Six, E., & Rausell, A. (2021). Gene signature extraction and cell identity recognition at the single-cell level with Cell-ID. Nature Biotechnology 2021 39:9, 39(9), 1095-1102.
Cuomo ASE, Seaton D D, McCarthy D J, Martinez I, Bonder M J, Garcia-Bernardo J, Amatya S, Madrigal P, Isaacson A, Buettner F, Knights A, Natarajan K N, HipSci Consortium, Vallier L, Marioni J C, Chhatriwala M, Stegle O. 2020. Single-cell RNA-sequencing of differentiating iPS cells reveals dynamic genetic effects on gene expression. Nature Communications 11:810.
del Bosque-Plata, L., Hernandez-Cortes, E. P., & Gragnoli, C. (2022). The broad pathogenetic role of TCF7L2 in human diseases beyond type 2 diabetes. Journal of Cellular Physiology, 237(1), 301-312.
Dey K K, Hsiao C J, Stephens M. 2017. Visualizing the structure of RNA-seq expression data using grade of membership models. PLOS Genetics 13:e1006599.
Dobin A, Davis C A, Schlesinger F, Drenkow J, Zaleski C, Jha S, Batut P, Chaisson M, Gingeras T R. 2013. STAR: ultrafast universal RNA-seq aligner. Bioinformatics 29:15-21.
Evseenko D, Zhu Y, Schenke-Layland K, Kuo J, Latour B, Ge S, Scholes J, Dravid G, Li X, MacLellan W R, Crooks G M. 2010. Mapping the first stages of mesoderm commitment during differentiation of human embryonic stem cells. PNAS 107:13742-13747.
GTEx Consortium. 2020. The GTEx Consortium atlas of genetic regulatory effects across human tissues. Science 369:1318-1330.
Gu Z, Eils R, Schlesner M. 2016. Complex heatmaps reveal patterns and correlations in multidimensional genomic data. Bioinformatics 32:2847-2849.
Guo H, Tian L, Zhang J Z, Kitani T, Paik D T, Lee W H, Wu J C. 2019. Single-Cell RNA Sequencing of Human Embryonic Stem Cell Differentiation Delineates Adverse Effects of Nicotine on Embryonic Development. Stem Cell Reports 12:772-7866.
Hafemeister C, Satija R. 2019. Normalization and variance stabilization of single-cell RNA-seq data using regularized negative binomial regression. Genome Biology 20:296.
Haghverdi L, Büttner M, Wolf F A, Buettner F, Theis F J. 2016. Diffusion pseudotime robustly reconstructs lineage branching. Nature Methods 13:845-848.
Han X, Chen H, Huang D, Chen H, Fei L, Cheng C, Huang H, Yuan G C, Guo G. 2018. Mapping human pluripotent stem cell differentiation pathways using high throughput single-cell RNA-sequencing. Genome Biology 19:47.
Han X, Zhou Z, Fei L, Sun H, Wang R, Chen Y, Chen H, Wang J, Tang H, Ge W, Zhou Y, Ye F, Jiang M, Wu J, Xiao Y, Jia X, Zhang T, Ma X, Zhang Q, Bai X, et al. 2020. Construction of a human cell landscape at single-cell level. Nature 581:303-309.
Hoffman G E, Schadt E E. 2016. variancePartition: interpreting drivers of variation in complex gene expression studies. BMC Bioinformatics 17:483.
Kang H M, Subramaniam M, Targ S, Nguyen M, Maliskova L, McCarthy E, Wan E, Wong S, Byrnes L, Lanata C M, Gate R E, Mostafavi S, Marson A, Zaitlen N, Criswell L A, Ye C J. 2018. Multiplexed droplet single-cell RNA-sequencing using natural genetic variation. Nature Biotechnology 36:89-94.
Karczewski, K. J., Francioli, L. C., Tiao, G., Cummings, B. B., Alföldi, J., Wang, Q., Collins, R. L., Laricchia, K. M., Ganna, A., Birnbaum, D. P., Gauthier, L. D., Brand, H., Solomonson, M., Watts, N. A., Rhodes, D., Singer-Berk, M., England, E. M., Seaby, E. G., Kosmicki, J. A., . . . MacArthur, D. G. (2020). The mutational constraint spectrum quantified from variation in 141,456 humans. Nature, 581(7809), Article 7809.
Korsunsky I, Millard N, Fan J, Slowikowski K, Zhang F, Wei K, Baglaenko Y, Brenner M, Loh P-R, Raychaudhuri S. 2019. Fast, sensitive and accurate integration of single-cell data with Harmony. Nature Methods 16:1289-1296.
Law C W, Chen Y, Shi W, Smyth G K. 2014. voom: Precision weights unlock linear model analysis tools for RNA-seq read counts. Genome Biology 15:R29.
Lun A T L, Riesenfeld S, Andrews T, Dao T P, Gomes T, participants in the 1st Human Cell Atlas Jamboree, Marioni J C. 2019. EmptyDrops: distinguishing cells from empty droplets in droplet-based single-cell RNA sequencing data. Genome Biology 20:63.
Ritchie M E, Phipson B, Wu D, Hu Y, Law C W, Shi W, Smyth G K. 2015. limma powers differential expression analyses for RNA-sequencing and microarray studies. Nucleic Acids Research 43:e47.
Sarkar A K, Tung P-Y, Blischak J D, Burnett J E, Li Y I, Stephens M, Gilad Y. 2019. Discovery and characterization of variance QTLs in human induced pluripotent stem cells. PLOS Genetics 15:e1008045
Strober B J, Elorbany R, Rhodes K, Krishnan N, Tayeb K, Battle A, Gilad Y. 2019. Dynamic genetic regulation of gene expression during cellular differentiation. Science 364:1287-1290.
Tanabe, A., Yanagiya, T., Iida, A., Saito, S., Sekine, A., Takahashi, A., Nakamura, T., Tsunoda, T., Kamohara, S., Nakata, Y., Kotani, K., Komatsu, R., Itoh, N., Mineo, I., Wada, J., Funahashi, T., Miyazaki, S., Tokunaga, K., Hamaguchi, K., . . . Hotta, K. (2007). Functional Single-Nucleotide Polymorphisms in the Secretogranin III (SCG3) Gene that Form Secretory Granules with Appetite-Related Neuropeptides Are Associated with Obesity. The Journal of Clinical Endocrinology & Metabolism, 92(3), 1145-1154.
Umans B, Battle A, Gilad Y. 2020. Where Are the Disease-Associated EQTLs? Trends in Genetics 8:2020.
Wolf F A, Hamey F K, Plass M, Solana J, Dahlin J S, Gottgens B, Rajewsky N, Simon L, Theis F J. 2019. PAGA: graph abstraction reconciles clustering with trajectory inference through a topology preserving map of single cells. Genome Biology 20:59.
Yao D W, O'Connor L J, Price A L, Gusev A. 2020. Quantifying genetic effects on disease mediated by assayed gene expression levels. Nature Genetics 52:626-633.
Zheng G X Y, Terry J M, Belgrader P, Ryvkin P, Bent Z W, Wilson R, Ziraldo S B, Wheeler T D, McDermott G P, Zhu J, Gregory M T, Shuga J, Montesclaros L, Underwood J G, Masquelier D A, Nishimura S Y, Schnall-Levin M, Wyatt P W, Hindson C M, Bharadwaj R, et al. 2017. Massively parallel digital transcriptional profiling of single cells. Nature Communications 8:1-12.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred aspects of this invention are described herein, including the best mode known to the inventors for carrying out the invention. Variations of those preferred aspects may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of analyzing the genome or transcriptome of a mammal, the method comprising:
(a) forming a first multi-tissue organoid from a first pluripotent stem cell (PSC) from a first mammal;
(b) forming a second multi-tissue organoid from a second PSC from a second mammal, wherein the second mammal is of the same species as the first mammal or of a different species than the first mammal;
(c) permitting each of the first multi-tissue organoid and the second multi-tissue organoid to differentiate;
(d) identifying cells within the first multi-tissue organoid and the second multi-tissue organoid by
(1) clustering cells and annotating the cells based on the genes that are highly expressed in each cluster,
(2) annotating cell type by correlation of gene expression using a reference data set of known primary cell types, or annotating cell state by correlation of gene expression using a reference data set of genes involved in known cells states, and
(3) applying topic modeling, matrix factorization, or grades of membership modelling to the annotated cells;
(e) performing single cell analysis on the first multi-tissue organoid and on the second multi-tissue organoid to generate data for the genome or transcriptome of the first multi-tissue organoid and to generate data for the genome or transcriptome of the second multi-tissue organoid,
the generated data being for the genome of both the first multi-tissue organoid and the second multi-tissue organoid or for the transcriptome of both the first multi-tissue organoid and the second multi-tissue organoid; and
(f) comparing the data of the first multi-tissue organoid to the data of the second multi-tissue organoid.

2. The method of claim 1, wherein the first PSC or second PSC is an induced PSC (iPSC).

3. The method of claim 1, wherein the data identifies association of a genetic variant with a molecular phenotype or an expression quantitative trait loci (eQTL).

4. The method of claim 1, wherein the single cell analysis is single cell RNA-sequencing (scRNA-seq) or single cell assay for transposase accessible chromatin (scATAC-seq).

5. The method of claim 1, wherein the method further comprises:
(i) forming a third multi-tissue organoid from a third PSC from a third mammal, wherein the third mammal is of the same species as the first mammal and the second mammal or of a different species than the first mammal and the second mammal;
(ii) permitting the third multi-tissue organoid to differentiate;
(iii) identifying cells within the third multi-tissue organoid by
(1) clustering cells and annotating the cells based on the genes that are highly expressed in each cluster,
(2) annotating cell type by correlation of gene expression using a reference data set of known primary cell types, or annotating cell state by correlation of gene expression using a reference data set of genes involved in known cells states, and
(3) applying topic modeling, matrix factorization, or grades of membership modelling to the annotated cells;
(iv) performing single cell analysis on the third multi-tissue organoid to generate data for the genome or transcriptome of the third multi-tissue organoid,
the generated data being for the genome of each of the first multi-tissue organoid, the second multi-tissue organoid, and the third multi-tissue organoid, or for the transcriptome of each of the first multi-tissue organoid, the second multi-tissue organoid, and the third multi-tissue organoid; and
(v) comparing the data of the third multi-tissue organoid to the data of the first multi-tissue organoid and to the data of the second multi-tissue organoid.

6. The method of claim 1, wherein identifying cells comprises applying topic modeling.

7. A method of classifying mammals, the method comprising:
(A) analyzing the genome or transcriptome of two or more mammals according to claim 1, wherein the analysis identifies a response quantitative trait loci (response QTL); and
(B) classifying the two or more mammals based on the response QTL.

8. A method of analyzing a disease-state of a mammal, the method comprising analyzing a genome or transcriptome of a mammal according to the method of claim 1, wherein the genome or transcriptome is associated with a disease-state.

9. The method of claim 1, wherein the data generated is for the genome of each of the multi-tissue organoids.

10. The method of claim 1, wherein the data generated is for the transcriptome of each of the multi-tissue organoids.

11. A method of analyzing a cellular response to a treatment, the method comprising:
(a) forming a first multi-tissue organoid from a first pluripotent stem cell (PSC) from a first mammal;
(b) forming a second multi-tissue organoid from a second PSC from the first mammal;
(c) permitting each of the first multi-tissue organoid and the second multi-tissue organoid to differentiate;
(d) identifying cells within the first multi-tissue organoid and the second multi-tissue organoid by
(1) clustering cells and annotating the cells based on the genes that are highly expressed in each cluster,
(2) annotating cell type by correlation of gene expression using a reference data set of known primary cell types, or annotating cell state by correlation of gene expression using a reference data set of genes involved in known cells states, and
(3) applying topic modeling, matrix factorization, or grades of membership modelling to the annotated cells;
(e) treating the first multi-tissue organoid with a substance while not treating the second multi-tissue organoid with the substance; and
(f) comparing the treatment of the first multi-tissue organoid to the untreated second multi-tissue organoid.

12. The method of claim 11, wherein the first PSC or second PSC is an induced PSC (iPSC).

13. The method of claim 11, wherein identifying cells comprises applying topic modeling.

14. The method of claim 11, further comprising
(i) forming a third multi-tissue organoid from a third pluripotent stem cell (PSC) from a second mammal, wherein the second mammal is of the same species as the first mammal or of a different species than the first mammal;
(ii) forming a fourth multi-tissue organoid from a fourth PSC from the second mammal;
(iii) permitting each of the third multi-tissue organoid and the fourth multi-tissue organoid to differentiate;
(iv) identifying cells within the third multi-tissue organoid and the fourth multi-tissue organoid by
(1) clustering cells and annotating the cells based on the genes that are highly expressed in each cluster,
(2) annotating cell type by correlation of gene expression using a reference data set of known primary cell types, or annotating cell state by correlation of gene expression using a reference data set of genes involved in known cells states, and
(3) applying topic modeling, matrix factorization, or grades of membership modelling to the annotated cells;
(v) treating the third multi-tissue organoid with a substance while not treating the fourth multi-tissue organoid with the substance; and
(vi) comparing the treatment of the third multi-tissue organoid to the untreated fourth multi-tissue organoid.

15. The method of claim 14, further comprising comparing (1) the treatment of the first multi-tissue organoid compared to the untreated second multi-tissue organoid to (2) the treatment of the third multi-tissue organoid compared to the untreated fourth multi-tissue organoid.

16. A method of determining an effect of a substance, the method comprising:
(a) forming two or more multi-tissue organoids, each organoid formed from a separate pluripotent stem cell (PSC) from a mammal;
(b) permitting each of the multi-tissue organoids to differentiate;
(c) identifying cells within each of the multi-tissue organoids by
(1) clustering cells and annotating the cells based on the genes that are highly expressed in each cluster,
(2) annotating cell type by correlation of gene expression using a reference data set of known primary cell types, or annotating cell state by correlation of gene expression using a reference data set of genes involved in known cells states, and
(3) applying topic modeling, matrix factorization, or grades of membership modelling to the annotated cells;
(d) treating the two or more multi-tissue organoids with an amount of the substance, wherein each of the multi-tissue organoids is treated with a different amount of the substance;
(f) comparing the two or more multi-tissue organoids; and
(g) determining an effect of the substance on the two or more multi-tissue organoids.

17. The method of claim 16, wherein the first PSC or second PSC is an induced PSC (iPSC).

18. The method of claim 16, further comprising:
(i) forming an additional multi-tissue organoid from an additional PSC from the mammal;
(ii) permitting the additional multi-tissue organoid to differentiate;
(iii) identifying cells within the additional multi-tissue organoid by
(1) clustering cells and annotating the cells based on the genes that are highly expressed in each cluster,
(2) annotating cell type by correlation of gene expression using a reference data set of known primary cell types, or annotating cell state by correlation of gene expression using a reference data set of genes involved in known cells states, and
(3) applying topic modeling, matrix factorization, or grades of membership modelling to the annotated cells;
(iv) leaving the additional multi-tissue organoid untreated with the substance; and
(v) comparing the two or more multi-tissue organoids to the additional multi-tissue organoid.

19. The method of claim 16, wherein comparing the two or more multi-tissue organoids to the additional multi-tissue organoid comprises assessing in at least one of the multi-tissue organoids cell death of one or more cell types.

20. The method of claim 19, wherein the assessment is of all cell types within the multi-tissue organoids; comprises using microscopy, live/dead cell staining, immunostaining, or cell counting; comprises using flow cytometry, qPCR, or single cell sequencing; comprises assessing expression of genes associated with cell stress or apoptosis; or comprises assessing on-target therapeutic effects on gene expression.

21. The method of claim 19, further comprising determining an optimal dosage, wherein the optimal dosage is the amount of the substance that is determined to cause reduced cell death and reduced stress response across all cell types and to cause on-target effects for a subset of cell types compared to other amounts of the substance.

22. The method of claim 16, wherein the substance is an environmental compound.

23. The method of claim 16, wherein identifying cells comprises applying topic modeling.

24. A method of treating a multi-tissue organoid, the method comprising:
(A) determining an optimal dosage of a substance according to claim 21;
(B) forming another multi-tissue organoid from a PSC from a mammal;
(C) permitting the multi-tissue organoid of (B) to differentiate; and
(D) treating the differentiated multi-tissue organoid of (C) with the dosage determined in (A).

25. A method of treating a mammal, the method comprising:
(A) determining an optimal dosage of a substance according to claim 21; and
(B) treating the mammal with the optimal dosage of the substance as determined in (A).

26. The method of claim 25, further comprising assessing in the treated mammal the health of a cell type that was adversely affected by the substance in a multi-tissue organoid during determination of the optimal dosage.

27. The method of claim 24, wherein identifying cells comprises applying topic modeling.

28. A method of analyzing a cellular response to a treatment, the method comprising:
(a) forming a first multi-tissue organoid from a first pluripotent stem cell (PSC) from a first mammal, wherein the first mammal has a disease-state;
(b) forming a second multi-tissue organoid from a second PSC from a second mammal, wherein the second mammal is of the same species as the first mammal or of a different species than the first mammal and the second mammal does not have the disease-state;
(c) permitting each of the first multi-tissue organoid and the second multi-tissue organoid to differentiate;
(d) identifying cells within the first multi-tissue organoid and the second multi-tissue organoid by
(1) clustering cells and annotating the cells based on the genes that are highly expressed in each cluster,
(2) annotating cell type by correlation of gene expression using a reference data set of known primary cell types, or annotating cell state by correlation of gene expression using a reference data set of genes involved in known cells states, and
(3) applying topic modeling, matrix factorization, or grades of membership modelling to the annotated cells;
(e) treating the first multi-tissue organoid and the second multi-tissue organoid with a substance; and
(f) comparing the treatment of the first multi-tissue organoid to the treatment of the second multi-tissue organoid.

29. The method of claim 28, wherein the first PSC or second PSC is an induced PSC (iPSC).

30. The method of claim 28, further comprising:
(i) forming an additional multi-tissue organoid from an additional PSC from the first mammal or the second mammal;
(ii) permitting the additional multi-tissue organoid to differentiate;
(iii) identifying cells within the additional multi-tissue organoid by
(1) clustering cells and annotating the cells based on the genes that are highly expressed in each cluster,
(2) annotating cell type by correlation of gene expression using a reference data set of known primary cell types, or annotating cell state by correlation of gene expression using a reference data set of genes involved in known cells states, and
(3) applying topic modeling, matrix factorization, or grades of membership modelling to the annotated cells;
(iv) leaving the additional multi-tissue organoid untreated with the substance; and
(v) comparing the treatment of the first multi-tissue organoid and the treatment of the second multi-tissue organoid to the additional multi-tissue organoid.

31. The method of claim 28, wherein comparing the treatment of the organoids comprises assessing in at least one of the multi-tissue organoids cell death of one or more cell types.

32. The method of claim 31, wherein the assessment is of all cell types within the multi-tissue organoids; comprises using microscopy, live/dead cell staining, immunostaining, or cell counting; comprises using flow cytometry, qPCR, or single cell sequencing; comprises assessing expression of genes associated with cell stress or apoptosis; or comprises assessing on-target therapeutic effects on gene expression.

33. The method of claim 28, wherein identifying cells comprises applying topic modeling.
